(19) United States
(12) Patent Application Publication
(10) Pub. No.: US 2025/0205356 A1
(43) Pub. Date: Jun. 26, 2025

(54) POLYMER-DRUG CONJUGATES

(71) Applicant: PRIMELINK BIOTHERAPEUTICS (SHENZHEN) CO., LTD, Shenzhen, Guangdong (CN)

(72) Inventors: Mao YIN, Needham, MA (US); Xianhua YI, Suzhou, Jiangsu (CN); Yingdong LU, Suzhou, Jiangsu (CN); Xiaohe SHI, Suzhou, Jiangsu (CN); Ling XIN, Suzhou, Jiangsu (CN); Guangchao ZHANG, Suzhou, Jiangsu (CN); Jiani YU, Suzhou, Jiangsu (CN); Yu SHEN, Suzhou, Jiangsu (CN)

(21) Appl. No.: 18/848,135

(22) PCT Filed: Mar. 20, 2023
(86) PCT No.: PCT/CN2023/082434

§ 371 Date: Sep. 18, 2024

(30) Foreign Application Priority Data
Mar. 21, 2022 (WO) PCT/CN2022/081885

(51) Int. Cl. A61K 47/68; A61K 47/54; A61K 47/59; A61K 47/60

(52) U.S. Cl. CPC A61K 47/6889; A61K 47/549; A61K 47/595; A61K 47/60; A61K 47/68037

(57) ABSTRACT

A polymeric scaffold useful for conjugating with a targeting moiety can form a targeting moiety-polymer-drug conjugate. A targeting moiety-polymer-drug conjugate is prepared from the polymeric scaffold. Compositions comprise the conjugates. Methods of their preparation and methods of treating various disorders with the conjugates or their compositions.

### TECHNICAL FIELD

The present disclosure generally relates to compounds, conjugates comprising these compounds, pharmaceutical compositions thereof, and method for the treatment of diseases or disorders with the conjugates or their pharmaceutical compositions.

### BACKGROUND OF THE DISCLOSURE

Traditionally, therapeutic agents primarily consisting of small molecules are delivered to the body via oral/GI absorption or systemic injection and then to the action site by the blood circulation. However, many challenges still remain to be addressed. For example, many therapeutic agents exhibit limited or otherwise reduced potencies and therapeutic effects because they are either generally subject to partial degradation before they reach a desired target in the body, or accumulate in tissues other than the target, or both.

Therefore, there is a need to deliver therapeutic agents intact to specifically targeted areas of the body through a system that can stabilize the drug and control the in vivo transfer of the therapeutic agent such that maximum cytotoxicity for the therapeutic agent is achieved.

### SUMMARY OF THE DISCLOSURE

The present disclosure relates to a polymeric scaffold delivery system that exhibits high drug load and strong binding to target antigen, thereby efficiently delivering and releasing the drugs to the target site. The present disclosure also relates to a polymeric scaffold useful to conjugate with a targeting moiety so as to obtain the polymeric scaffold delivery system.

In one aspect, the present disclosure provides a polymeric scaffold of Formula (I) useful to conjugate with a targeting moiety:

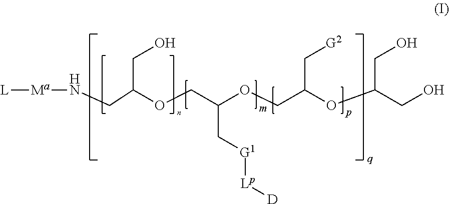

wherein:
   the polymeric scaffold comprises linear polyglycerol;
   L is a linking moiety comprising a functional group Wₚ that is capable of forming a covalent bond with the targeting moiety;
   M^{a} is a stretcher connecting L to —NH— moiety;
   each G¹ is independently a functional group connecting LP to the linear polyglycerol;
   each L^{p} is independently a drug release mechanism between a therapeutic agent moiety D and G¹;
   each D is independently a therapeutic agent moiety;
   each G² is independently a functional group capable of converting into a charged state;
   n is an integer from 0 to 1000;
   m is an integer from 0 to 1000;
   p is an integer from 0 to 1000; and
   q is an integer from 0 to 1000.

In one another aspect, the present disclosure provides a polymeric scaffold of Formula (II):

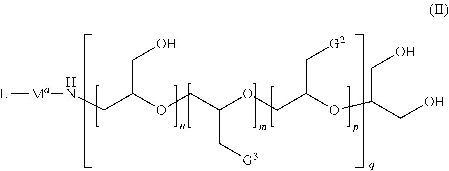

wherein,
   the polymeric scaffold comprises linear polyglycerol;
   each G² is independently a functional group capable of converting into a charged state;
   each G³ independently comprises a functional group capable of reacting with a reactive group in a drug release mechanism to connect the drug release mechanism to the linear polyglycerol;
   n is an integer from 0 to 1000;
   m is an integer from 0 to 1000;
   p is an integer from 0 to 1000; and
   q is an integer from 0 to 1000.

In one another aspect, the present disclosure provides a polymeric scaffold of Formula (III):

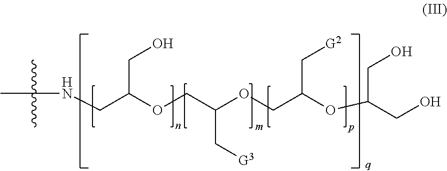

wherein,
   the polymeric scaffold comprises linear polyglycerol;
   each G² is independently a functional group capable of converting into a charged state;
   each G³ independently comprises a functional group capable of reacting with a reactive group in a drug release mechanism to connect the drug release mechanism to the linear polyglycerol;
   n is an integer from 0 to 1000;
   m is an integer from 0 to 1000;
   p is an integer from 0 to 1000; and
   q is an integer from 0 to 1000.

In one another aspect, the present disclosure provides a polymeric scaffold of Formula (IV):

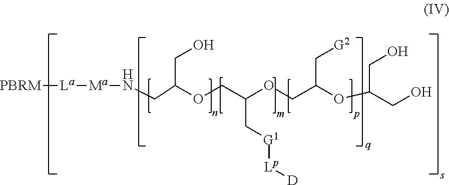

wherein,
   the polymeric scaffold comprises linear polyglycerol;
   each L^{a} is independently a divalent moiety connecting the targeting moiety to M^{a};
   each M^{a} is independently a stretcher connecting L^{a} to —NH— moiety;
   each G¹ is independently a functional group connecting L^{p} to the linear polyglycerol;
   each L^{p} is independently a drug release mechanism between a therapeutic agent moiety D and G¹;
   each D is independently a therapeutic agent moiety;
   each G² is independently a functional group capable of converting into a charged state;
   n is an integer from 0 to 1000;
   m is an integer from 0 to 1000;
   p is an integer from 0 to 1000;
   q is an integer from 0 to 1000; and
   s is an integer from 1 to 8.

In a further aspect, the present disclosure provides a pharmaceutical composition comprising a polymeric scaffold or conjugate described herein and a pharmaceutically acceptable carrier.

In another aspect, the present disclosure provides a method of treating diseases in a subject in need thereof, comprising administering to the subject a therapeutic effective amount of the polymeric scaffold or conjugate described herein, or the pharmaceutical composition provided herein.

### DESCRIPTION OF DRAWINGS

FIG. 1 shows the cell based binding of Trastuzumab and the test anti-HER2 ADCs to HER2 positive SKBR-3 cells (A) and NCI-N87 cells (B).

FIG. 2 shows the internalization of the test anti-HER2 ADCs into HER2 positive SKBR-3 cells (A) and NCI-N87 cells (B) on 0 to 24 hours.

FIG. 3 shows the in vitro cytotoxicity of the test anti-HER2 ADCs on HER2 expressing SKBR-3 cells (A) and NCI-N87 cells (B), as well as HER2 negative MCF-7 cells (C).

FIG. 4 shows the cell based binding of Trop2 antibodies Trop2-1, Trop2-2, and Datopotamab, as well as exemplary anti-Trop2 ADCs to Trop2 positive SKBR-3 cells.

FIG. 5 shows the internalization of the test anti-Trop2 ADCs into Trop2 positive SKBR-3 cells.

FIG. 6 shows the in vitro cytotoxicity of the test anti-Trop2 ADCs on Trop2 expressing SKBR-3 cells (A), NCI-N87 cells (B), MCF-7 cells (C), and MDA-MB-468 cells (D).

FIG. 7 shows that ADC-5 and ADC-6 is efficacious in inhibiting tumor growth in established NCI-N87 tumor cell xenograft BALB/c Nude mice. (A) Tumor volume change in the 28-day treatment. (B) Body weight change in the 28-day treatment. (C) Tumor weight measured at day 28 after administration.

### DETAILED DESCRIPTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination.

It must be noted that, as used in the specification and the appended claims, the singular forms “a,” “an,” and “the” include plural forms of the same unless the context clearly dictates otherwise. In this specification and in the claims that follow, reference will be made to a number of terms that shall be defined to have the following meanings unless a contrary intention is apparent.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modem Methods of Organic Synthesis, 3^{rd} Edition, Cambridge University Press, Cambridge, 1987; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two R moieties and Rⁱ at each occurrence is selected independently from the definition of Rⁱ. Also, combinations of substituents and/or variables are permissible, but only if such combinations result in stable compounds.

As used herein, a dash “—” at the front or end of a chemical group is used, a matter of convenience, to indicate a point of attachment for a substituent. For example, —OH is attached through the carbon atom; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. As used herein, a solid line coming out of the center of a ring indicates that the point of attachment for a substituent on the ring can be at any ring atom. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

The term “about”, when used in connection with a numerical value, means that a collection or range of values is included. For example, “about X” includes a range of values that are ±20%, ±10%, ±5%, ±2%, ±1%, ±0.5%, ±0.2%, or ±0.1% of X, where X is a numerical value. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “n is an integer between 1 and 6” and “n being an integer of 1 to 6” both mean “x being 1, 2, 3, 4, 5, or 6”.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms. In similar manner, the term “m-n membered” ring, wherein m and n are integers and n is greater than m, refers to a ring containing m to n atoms.

As used herein, the term “aliphatic” includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to a linear or branched-chain alkyl having i to j carbon atoms. For example, alkyl groups contain 1 to 10, 1 to 9, 1 to 8, 1 to 7, 1 to 6, 1 to 5, 1 to 4, 1 to 3, or 1 to 2 carbon atoms. Examples of “C₁₋₆ alkyl” include, but are not limited to, methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 2-ethyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “amino” refers to the group —NR^{a}R^{b}, wherein R^{a} and R^{b} are independently selected from groups consisting of hydrogen, alkyl, haloalkyl, aryl, heteroaryl, cycloalkyl, and heterocyclyl and each of which may be optionally substituted.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like.

As used herein, the term “cycloalkyl”, whether as part of another term or used independently, refers to a monovalent non-aromatic, saturated or partially unsaturated monocyclic and polycyclic ring system, in which all the ring atoms are carbon and which contains at least three ring forming carbon atoms. In some embodiments, the cycloalkyl group may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. The cycloalkyl group may be saturated or partially unsaturated. In some embodiments, the cycloalkyl group may be a saturated cyclic alkyl group. In some embodiments, the cycloalkyl group may be a partially unsaturated cyclic alkyl group that contains at least one double bond or triple bond in its ring system.

In some embodiments, the cycloalkyl group may be saturated or partially unsaturated monocyclic carbocyclic ring system, examples of which include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl.

In some embodiments, the cycloalkyl group may be saturated or partially unsaturated polycyclic (e.g., bicyclic and tricyclic) carbocyclic ring system, which can be arranged as a fused, spiro or bridged ring system. As used herein, the term “fused ring” refers to a ring system having two rings sharing two adjacent atoms, the term “spiro ring” refers to a ring systems having two rings connected through one single common atom, and the term “bridged ring” refers to a ring system with two rings sharing three or more atoms. Examples of fused carbocyclyl include, but are not limited to, naphthyl, benzopyrenyl, anthracenyl, acenaphthenyl, fluorenyl and the like. Examples of spiro carbocyclyl include, but are not limited to, spiro[5.5]undecanyl, spiro-pentadienyl, spiro[3.6]-decanyl, and the like. Examples of bridged carbocyclyl include, but are not limited to bicyclo[1,1,1]pentenyl, bicyclo[2,2,1]heptenyl, bicyclo[2.2.1]heptanyl, bicyclo[2.2.2]octanyl, bicyclo[3.3.1]nonanyl, bicyclo[3.3.3]undecanyl, and the like.

As used herein, the term “halo” or “halogen” refers to an atom selected from fluorine (or fluoro), chlorine (or chloro), bromine (or bromo) and iodine (or iodo).

As used herein, the term “heteroatom” refers to nitrogen, oxygen, sulfur or phosphorus, and includes any oxidized form of nitrogen, sulfur or phosphorus, and any quaternized form of a basic nitrogen.

As used herein, the term “heteroaliphatic” refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be branched or linear unbranched. As will be appreciated by one of ordinary skill in the art, “heteroaliphatic” is intended herein to include, but is not limited to, heteroalkyl, heteroalkenyl, heteroalkynyl moieties. In certain embodiments, heteroaliphatic moieties are substituted (“substituted heteroaliphatic”) by independent replacement of one or more of the hydrogen atoms thereon with one or more moieties including, but not limited to aliphatic; heteroaliphatic; cycloalkyl; heterocycloalkyl; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHC₁₂; —CH₂OH; —CH₂CH₂OH; —CH₂NH₂; —CH₂SO₂CH₃; — or -GR^{G1}, wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO2-, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, cycloalkyl, heterocycloalkyl, aryl, heteroaryl, alkylaryl, or alkylheteroaryl moiety. Additional examples of generally applicable substituents are illustrated by the specific embodiments shown in the Examples that are described herein.

As used herein, the term “heteroalkyl” refers to an alkyl, at least one of the carbon atoms of which is replaced with a heteroatom selected from N, O, or S. The heteroalkyl may be a carbon radical or heteroatom radical (i.e., the heteroatom may appear in the middle or at the end of the radical), and may be optionally substituted independently with one or more substituents described herein. The term “heteroalkyl” encompasses alkoxyl and heteroalkoxy radicals.

As used herein, the term “heteroalkenyl” refers to an alkenyl, at least one of the carbon atoms of which is replaced with a heteroatom selected from N, O, or S. The heteroalkenyl may be a carbon radical or heteroatom radical (i.e., the heteroatom may appear in the middle or at the end of the radical), and may be optionally substituted independently with one or more substituents described herein.

As used herein, the term “heteroalkynyl” refers to an alkynyl, at least one of the carbon atoms of which is replaced with a heteroatom selected from N, O, or S. The heteroalkynyl may be a carbon radical or heteroatom radical (i.e., the heteroatom may appear in the middle or at the end of the radical), and may be optionally substituted independently with one or more substituents described herein.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, heteroaryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocycloalkyl” refers to a saturated or partially unsaturated cycloalkyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocycloalkyl is a saturated heterocycloalkyl. In some embodiments, the heterocycloalkyl is a partially unsaturated heterocycloalkyl having one or more double bonds in its ring system. In some embodiments, the heterocycloalkyl may contains any oxidized form of carbon, nitrogen or sulfur, and any quaternized form of a basic nitrogen. The heterocycloalkyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

Heterocycloalkyl group may be monocyclic. Examples of monocyclic heterocycloalkyl include, but are not limited to oxetanyl, 1,1-dioxothietanylpyrrolidyl, tetrahydrofuryl, tetrahydropyranyl, tetrahydrothienyl, azetidinyl, pyrrolyl, furanyl, thienyl, pyrazolyl, imidazolyl, triazolyl, oxazolyl, thiazolyl, piperidyl, piperazinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like.

Heterocycloalkyl group may be polycyclic, including the fused, spiro and bridged ring systems. The fused heterocycloalkyl group includes radicals wherein the heterocycloalkyl radicals are fused with a saturated, partially unsaturated, or fully unsaturated (i.e., aromatic) carbocyclic or heterocyclic ring. Examples of fused heterocycloalkyl include, but are not limited to, phenyl fused ring or pyridinyl fused ring, such as quinolinyl, isoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, furo[3,4-d]pyrimidinyl, pyrrolo[3,4-d]pyrimidinyl, dihydrofuro[3,4-b]pyridinyl groups, and the like. Examples of spiro heterocycloalkyl include, but are not limited to, spiropyranyl, spirooxazinyl, 5-aza-spiro[2.4]heptanyl, 6-aza-spiro[2.5]octanyl, 6-aza-spiro[3.4]octanyl, 2-oxa-6-aza-spiro[3.3]heptanyl, 2-oxa-6-aza-spiro[3.4]octanyl, 6-aza-spiro[3.5]nonanyl, 7-aza-spiro[3.5]nonanyl, 1-oxa-7-aza-spiro[3.5]nonanyl, 3,8-dioxa-1-azaspiro[4.5]dec-1-enyl and the like. Examples of bridged heterocycloalkyl include, but are not limited to, 3-aza-bicyclo[3.1.0]hexanyl, 8-aza-bicyclo[3.2.1]octanyl, 1-aza-bicyclo[2.2.2]octanyl, 2-aza-bicyclo[2.2.1]heptanyl, 1,4-diazabicyclo[2.2.2]octanyl, and the like.

As used herein, the term “hydroxyl” refers to —OH.

As used herein, the term “leaving group” refers to a molecular fragment that departs with a pair of electrons in heterolytic bond cleavage. Leaving groups can be anions or neutral molecules. Leaving groups include, but are not limited to halides such as Cl⁻, Br⁻, and I⁻, sulfonate esters, such as para-toluenesulfonate (“tosylate”, TsO⁻), and RC(O)O⁻ in which R is hydrogen, aliphatic, heteroaliphatic, cycloalkyl, or heterocycloalkyl moiety.

As used herein, the term “optional” or “optionally” means that the subsequently described event or circumstance may or may not occur, and that the description includes instances where the said event or circumstance occurs and instances in which it does not.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “protecting group” means that a particular functional moiety, e.g., O, S, or N, is temporarily blocked so that a reaction can be carried out selectively at another reactive site in a multifunctional compound. In some embodiments, a protecting group reacts selectively in good yield to give a protected substrate that is stable to the projected reactions. The protecting group must be selectively removed in good yield by readily available, preferably nontoxic reagents that do not attack the other functional groups. The protecting group forms an easily separable derivative (more preferably without the generation of new stereogenic centers). The protecting group has a minimum of additional functionality to avoid further sites of reaction. As detailed herein, oxygen, sulfur, nitrogen and carbon protecting groups may be utilized. For example, in some embodiments, certain exemplary oxygen protecting groups may be utilized. These oxygen protecting groups include, but are not limited to methyl ethers, substituted methyl ethers (e.g., MOM (methoxymethyl ether), MTM (methylthiomethyl ether), BOM (benzyloxymethyl ether), and PMBM (p-methoxybenzyloxymethyl ether)), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, and TBDPS (t-butyldiphenyl silyl ether), esters (e.g., formate, acetate, benzoate (Bz), trifluoroacetate, and dichloroacetate), carbonates, cyclic acetals and ketals. In some other embodiments, nitrogen protecting groups are utilized. Nitrogen protecting groups, as well as protection and deprotection methods are known in the art. Nitrogen protecting groups include, but are not limited to, carbamates (including methyl, ethyl and substituted ethyl carbamates (e.g., Troc), amides, cyclic imide derivatives, N-Alkyl and N-Aryl amines, imine derivatives, and enamine derivatives. In yet other embodiments, certain exemplary sulphur protecting groups may be utilized. The sulfur protecting groups include, but are not limited to those oxygen protecting group describe above as well as aliphatic carboxylic acid (e.g., acrylic acid), maleimide, vinyl sulfonyl, and optionally substituted maleic acid. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

As used herein, the term “leaving group” refers to a molecular fragment that departs with a pair of electrons in heterolytic bond cleavage. Leaving groups can be anions or neutral molecules. Leaving groups include, but are not limited to halides such as Cl⁻, Br⁻, and I⁻, sulfonate esters, such as para-toluenesulfonate (“tosylate”, TsO⁻), and RC(O)O⁻ in which R is hydrogen, an aliphatic, heteroaliphatic, carbocyclic, or heterocycloalkyl moiety.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. The substituents may include, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

As used herein, the term “targeting moiety” refers to a molecule that recognizes and binds to a cell surface marker or receptor such as, a transmembrane protein, surface immobilized protein, or protoglycan. Examples of targeting moiety include but are not limited to, antibodies or fragment thereof, lipocalins, proteins, peptides or peptide mimics, and the like. The targeting moiety, in addition to targeting the polymeric scaffold to a specific cell, tissue or location, may also have certain therapeutic effect such as antiproliferative (cytostatic and/or cytotoxic) activity against a target cell or pathway. The targeting moiety comprises or may be engineered to comprise at least one chemically reactive group such as, —COOR, —SH, amine or a chemically reactive amino acid moiety or side chains such as, for example, tyrosine, histidine, cysteine, or lysine. In some embodiments, a targeting moiety may be a ligand which specifically binds or complexes with a cell surface molecule, such as a cell surface receptor or antigen, for a given target cell population. Following specific binding or complexing of the ligand with its receptor, the cell is permissive for uptake of the ligand or ligand-drug-conjugate, which is then internalized into the cell. As used herein, a ligand that “specifically binds or complexes with” or “targets” a cell surface molecule preferentially associates with a cell surface molecule via intermolecular forces.

As used herein, the term “ligand” refers to a variety of chemical or biological molecules, which can have a specific binding affinity to a selected target, wherein the selected target can be, for example, a cell surface receptor, a cell surface antigen, a cell, a tissue, an organ, etc. In some embodiments, the ligand can specifically bind to a protein or a marker expressed on the surface of a target cell. In some embodiments, the ligand of the present disclosure binds to a cell surface protein or marker with an affinity of 10⁶-10⁻¹¹ M (K_{d} value). In some embodiments, the ligand of the present disclosure binds to a cell surface protein or marker with an affinity of at least 10⁻⁷, at least 10⁻⁸ and at least 10⁻⁹ M (K_{d} value). In some embodiments, the ligand of the present disclosure binds to a cell surface protein or marker with an affinity of less than 10⁻⁶, less than 10⁻⁷ and less than 10⁻⁸ M (K_{d} value). In some embodiments, the ligand of the present disclosure binds to a cell surface protein or marker with a certain affinity, wherein the certain affinity refers to the affinity of the ligand to a target cell surface protein or marker which is at least two, three, four, five, six, eight, ten, twenty, fifty, one hundred or more times higher than that to a non-target cell surface protein or marker. In some embodiments, the expression of the cell surface protein or marker of the present disclosure in target cells (e.g. cancer cells) is significantly higher than that in normal cells. The term “significantly” as used herein refers to statistically significant differences, or significant differences that can be recognized by a person skilled in the art.

As used herein, the term “targeting moiety” refers to a molecule, complex, or aggregate, that binds specifically or selectively to a target molecule, cell, particle, tissue or aggregate. Examples of targeting moiety includes, but are not limited to antibody, antibody binding fragment, bispecific antibody, immunoglobins or other antibody-based molecule or compound. However, other examples of targeting moieties are known in the art and may be used, such as aptamers, avimers, receptor-binding ligands, nucleic acids, biotin-avidin binding pairs, peptides, small molecules, nanoparticles or proteins, etc. The terms “targeting moiety” and “binding moiety” are used synonymously herein.

As used herein, the term “drug” refers to a compound which is biologically active and provides a desired physiological effect following administration to a subject in need thereof (e.g., an active pharmaceutical ingredient).

As used herein, the term “antibody” includes any immunoglobulin, monoclonal antibody, polyclonal antibody, multivalent antibody, multispecific antibody, or bispecific (bivalent) antibody or a functional portion thereof that binds to a specific antigen. A native intact antibody comprises two heavy chains (H) and two light (L) chains inter-connected by disulfide bonds. Each heavy chain consists of a variable region (VH) and a first, second, and third constant region (CH1, CH2 and CH3, respectively), while each light chain consists of a variable region (VL) and a constant region (CL). Mammalian heavy chains are classified as α, δ, ε, γ, and μ, and mammalian light chains are classified as λ or κ. The variable regions of the light and heavy chains are responsible for antigen binding. The variables region in both chains are generally subdivided into three regions of hypervariability called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Therefore, each VH and VL comprises of three CDRs and four FRs in the following order (amino acid residues N terminus to C terminus): FR1, CDR1, FR2, CDR2, FR3, CDR3, FR4. The constant regions of the heavy and light chains are not involved in antigen binding, but exhibit various effector functions. Antibodies are assigned to the five major classes based on the amino acid sequence of the constant region of their heavy chain: IgA, IgD, IgE, IgG, and IgM, which are characterized by the presence of α, δ, ε, γ, and μ heavy chains, respectively. Subclasses of several of the major antibody classes are such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

As used herein, the term “Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

As used herein, the term “Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

As used herein, the term “F(ab′)₂” refers to a dimer of Fab′ that comprises two light chains and part of two heavy chains.

As used herein, the term “Fc” with regard to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, and CDC, but does not function in antigen binding.

As used herein, the term “Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

As used herein, the term “single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879 (1988)). A “scFv dimer” refers to a single chain comprising two heavy chain variable regions and two light chain variable regions with a linker. An “scFv dimer” may be a bivalent diabody or bivalent ScFv (BsFv) comprising V_{H}-V_{L} (linked by a peptide linker) dimerized with another V_{H}-V_{L} moiety such that V_{H}'S of one moiety coordinate with the V_{L}'S of the other moiety and form two binding sites which can target the same antigens (or eptipoes) or different antigens (or eptipoes). A “scFv dimer” may also be a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

As used herein, the term “single-chain Fv-Fc antibody” or “scFv-Fc” refers to an engineered antibody consisting of a scFv connected to the Fc region of an antibody.

As used herein, the term “camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)). “Diabodies” include small antibody fragments with two antigen-binding sites, wherein the fragments comprise a V_{H} domain connected to a V_{L} domain in a single polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

As used herein, the term “domain antibody” refers to an antibody fragment containing only the variable region of a heavy chain or the variable region of a light chain. In some embodiments, two or more V_{H} domains are covalently joined with a peptide linker to form a bivalent or multivalent domain antibody. The two V_{H} domains of a bivalent domain antibody may target the same or different antigens.

As used herein, the term “(dsFv)₂” refers to an antigen binding fragment consisting of three peptide chains: two V_{H} moieties linked by a peptide linker and bound by disulfide bridges to two V_{L} moieties.

As used herein, the term “bispecific ds diabody” refers to an antigen binding fragment consisting of V_{H1}-V_{L2} (linked by a peptide linker) bound to V_{L1}-V_{H2} (also linked by a peptide linker) via a disulfide bridge between V_{H1} and V_{L1}.

As used herein, the term “bispecific dsFv” or “dsFv-dsFv′” refers to a antigen binding fragment consisting of three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

In some embodiment, the antibody or its antigen binding fragment is chimeric or humanized.

As used herein, the term “chimeric” refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

As used herein, the term “humanized”, with reference to antibody or antigen-binding fragment, refers to the antibody or the antigen-binding fragment comprises CDRs derived from non-human animals (e.g. a rodent, rabbit, dog, goat, horse, or chicken), FR regions derived from human, and when applicable, the constant regions derived from human. In some embodiments, the constant regions from a human antibody are fused to the non-human variable regions. A humanized antibody or antigen-binding fragment is useful as human therapeutics. In some embodiments, the non-human animal is a mammal, for example, a mouse, a rat, a rabbit, a goat, a sheep, a guinea pig, a hamster, or a non-human primate (for example, a monkey (e.g., cynomolgus or rhesus monkey) or an ape (e.g., chimpanzee, gorilla, simian or affen)). In some embodiments, the humanized antibody or antigen-binding fragment is composed of substantially all human sequences except for the CDR sequences which are non-human. In some embodiments, the humanized antibody or antigen-binding fragment is modified to improve the antibody performance, such as binding or binding affinity. For example, one or more amino acid residues in one or more non-human CDRs are altered to reduce potential immunogenicity in human, wherein the altered amino acid residues either are not critical for immunospecific binding or the alterations are conservative changes, such that the binding of the humanized antibody to the antigen is not significantly affected. In some embodiments, the FR regions derived from human may comprise the same amino acid sequence as the human antibody from which it is derived, or it may comprise some amino acid changes, for example, no more than 10, 9, 8, 7, 6, 5, 4, 3, 2, or 1 changes of amino acid. In some embodiments, such change in amino acid could be present in heavy chain FR regions only, in light chain FR regions only, or in both chains.

As used herein, the term “natural amino acid” refers to any one of the common, naturally occurring L-amino acids found in naturally occurring proteins: glycine (Gly), alanine (Ala), valine (Val), leucine (Leu), isoleucine (Ile), lysine (Lys), arginine (Arg), histidine (His), proline (Pro), serine (Ser), threonine (Thr), phenylalanine (Phe), tyrosine (Tyr), tryptophan (Trp), aspartic acid (Asp), glutamic acid (Glu), asparagine (Asn), glutamine (Gln), cysteine (Cys) and methionine (Met).

As used herein, the term “non-natural amino acid” as used herein refers to any amino acid which is not a natural amino acid. This includes, for example, amino acids that comprise α-, β-, ω-, D-, L-amino acyl residues. More generally, the non-natural amino acid comprises a residue of the general formula

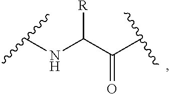

wherein the side chain R is other than the amino acid side chains occurring in nature. Exemplary unnatural amino acids, include, but are not limited to, sarcosine (N-methylglycine), citrulline (cit), homocitrulline, β-ureidoalanine, thiocitrulline, hydroxyproline, allothreonine, pipecolic acid (homoproline), α-aminoisobutyric acid, tert-butylglycine, tert-butylalanine, allo-isoleucine, norleucine, α-methylleucine, cyclohexylglycine, β-cyclohexylalanine, β-cyclopentylalanine, α-methylproline, phenylglycine, α-methylphenylalanine and homophenylalanine.

As used herein, the term “polypeptide”, “protein” or “peptide” can be a single amino acid or a polymer of amino acids. The polypeptide, protein or peptide as described in the present disclosure may contain naturally-occurring amino acids and non-naturally-occurring amino acids, or analogs and mimetics thereof. The polypeptide, protein or peptide can be obtained by any method well known in the art, for example, but not limited to, by an isolation and a purification from natural materials, a recombinant expression, a chemical synthesis, etc.

As used herein, the term “biocompatible” as used herein is intended to describe compounds that exert minimal destructive or host response effects while in contact with body fluids or living cells or tissues. Thus, a biocompatible group, as used herein, refers to an aliphatic, cycloalkyl, heteroaliphatic, heterocycloalkyl, aryl, or heteroaryl moiety, which falls within the definition of the term biocompatible, as defined above and herein. The term “biocompatibility” as used herein, is also taken to mean that the compounds exhibit minimal interactions with recognition proteins, e.g., naturally occurring antibodies, cell proteins, cells and other components of biological systems, unless such interactions are specifically desirable. Thus, substances and functional groups specifically intended to cause the above minimal interactions, e.g., drugs and prodrugs, are considered to be biocompatible. In some embodiments, compounds are “biocompatible” if their addition to normal cells in vitro, at concentrations similar to the intended systemic in vivo concentrations, results in less than or equal to 1% cell death during the time equivalent to the half-life of the compound in vivo (e.g., the period of time required for 50% of the compound administered in vivo to be eliminated/cleared), and their administration in vivo induces minimal and medically acceptable inflammation, foreign body reaction, immunotoxicity, chemical toxicity and/or other such adverse effects. As used herein, the term “normal cells” refers to cells that are not intended to be destroyed or otherwise significantly affected by the compound being tested.

As used herein, “biodegradable” polymers are polymers that are susceptible to biological processing in vivo. As used herein, “biodegradable” compounds or moieties are those that, when taken up by cells, can be broken down by the lysosomal or other chemical machinery or by hydrolysis into components that the cells can either reuse or dispose of without significant toxic effect on the cells. The term “biocleavable” as used herein has the same meaning of “biodegradable”. The degradation fragments preferably induce little or no organ or cell overload or pathological processes caused by such overload or other adverse effects in vivo. Examples of biodegradation processes include enzymatic and non-enzymatic hydrolysis, oxidation and reduction. Suitable conditions for non-enzymatic hydrolysis of the biodegradable protein-polymer-drug conjugates (or their components, e.g., the biodegradable polymeric carrier and the linkers between the carrier and the antibody or the drug molecule) described herein, for example, include exposure of the biodegradable conjugates to water at a temperature and a pH of lysosomal intracellular compartment. Biodegradation of some protein-polymer-drug conjugates (or their components, e.g., the biodegradable polymeric carrier and the linkers between the carrier and the antibody or the drug molecule), can also be enhanced extracellularly, e.g. in low pH regions of the animal body, e.g. an inflamed area, in the close vicinity of activated macrophages or other cells releasing degradation facilitating factors. In certain embodiments, the effective size of the polymer carrier at pH˜7.5 does not detectably change over 1 to 7 days, and remains within 50% of the original polymer size for at least several weeks. At pH˜5, on the other hand, the polymer carrier preferably detectably degrades over 1 to 5 days, and is completely transformed into low molecular weight fragments within a two-week to several-month time frame. Polymer integrity in such tests can be measured, for example, by size exclusion HPLC. Although faster degradation may be in some cases preferable, in general it may be more desirable that the polymer degrades in cells with the rate that does not exceed the rate of metabolization or excretion of polymer fragments by the cells. In certain embodiments, the polymers and polymer biodegradation byproducts are biocompatible.

As used herein, the term “bioavailability” refers to the systemic availability (i.e., blood/plasma levels) of a given amount of drug or compound administered to a subject. Bioavailability is an absolute term that indicates measurement of both the time (rate) and total amount (extent) of drug or compound that reaches the general circulation from an administered dosage form.

As used herein, the term “drug release mechanism” refers to a linking moiety that is biocleavable/biodegradable under intracellular conditions, such that the cleavage of the linking moiety release the drug in the intracellular environment. In some embodiments, the linking moiety is hydrolytically labile in water or in aqueous solutions including for example, body fluid such as blood, i.e., sensitive to hydrolysis at certain pHs. In some embodiments, the linking moiety is enzymatically labile, i.e., degradable by one or more enzymes. In some embodiments, the linking moiety is photo labile and is useful at the body surface and in many body cavities that are accessible to light. In some embodiments, the linking moiety is biocleavable under reducing conditions under which the activity of drug is not affected.

As used herein, the term “therapeutic agent” or “drug” refers to a compound which is biologically active and provides a desired physiological effect following administration to a subject in need thereof (e.g., an active pharmaceutical ingredient). In some embodiments, the therapeutic agent is small molecule drug.

As used herein, the term “small molecule” refers to molecules, whether naturally-occurring or artificially created (e.g., via chemical synthesis) that have a relatively low molecular weight. Preferred small molecules are biologically active in that they produce a local or systemic effect in animals, such as mammals, for example humans. In certain embodiments, the small molecule is a drug and the small molecule is referred to as “drug molecule” or “drug” or “therapeutic agent”. In certain embodiments, the drug molecule has MW less than or equal to about 5 kDa. In other embodiments, the drug molecule has MW less than or equal to about 1.5 kDa.

Classes of drug molecules that can be used in the present disclosure include, but are not limited to, anti-cancer substances, radionuclides, vitamins, anti-AIDS substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, imaging agents.

In certain embodiments, large molecules can be used as the therapeutic agents in the present disclosure. Examples of suitable large molecules include, but are not limited to, amino acid-based molecules, such as peptides, polypeptides, enzymes, antibodies, immunoglobulins, or functional fragments thereof, among others.

In some embodiments, the therapeutic agent used in the present disclosure is a therapeutic agent that has antiproliferative (cytostatic and/or cytotoxic) activity against a target cell or pathway. The drug may have a chemically reactive group such as, for example, —COOH, primary amine, secondary amine —NHR, —OH, —SH, —C(O)H, —C(O)R, —C(O)NHR′, —C(S)OH, —S(O)₂OR′, —P(O)₂OR′, —CN, —NC or —ONO, in which R is aliphatic, heteroaliphatic, carbocyclic or heterocycloalkyl moiety and R′ is a hydrogen, aliphatic, heteroaliphatic, cycloalkyl, heterocycloalkyl, aryl or heteroaryl moiety.

As used herein the term “cytotoxic” means toxic to cells or a selected cell population (e.g., cancer cells). The toxic effect may result in cell death and/or lysis. In certain instances, the toxic effect may be a sublethal destructive effect on the cell, e.g., slowing or arresting cell growth. In order to achieve a cytotoxic effect, the drug or prodrug may be selected from a group consisting of a DNA damaging agent, a microtubule disrupting agent, or a cytotoxic protein or polypeptide, amongst others.

As used herein, the term “specific binding” or “specifically binds” refers to a non-random binding reaction between two molecules, such as for example between an antibody and an antigen. In some embodiments, the antibodies or antigen-binding fragments provided herein specifically bind to a target antigen with a binding affinity (K_{D}) of about 0.01 nM to about 100 nM, about 0.1 nM to about 100 nM, 0.01 nM to about 10 nM, about 0.1 nM to about 10 nM, 0.01 nM to about 1 nM, about 0.1 nM to about 1 nM or about 0.01 nM to about 0.1 nM) at pH 7.4. K_{D} as used herein refers to the ratio of the dissociation rate to the association rate (k_{off}/kₒₙ), may be determined using surface plasmon resonance methods for example using instrument such as Biacore.

As used herein, the term “tumor antigen” refers to an antigenic substance produced in tumor cells, i.e., it triggers an immune response in the host. Normal proteins in the body are not antigenic because of self-tolerance, a process in which self-reacting cytotoxic T lymphocytes (CTLs) and autoantibody-producing B lymphocytes are culled “centrally” in primary lymphatic tissue (BM) and “peripherally” in secondary lymphatic tissue (mostly thymus for T-cells and spleen/lymph nodes for B cells). Thus, any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation.

As used herein, the term “effective amount” refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, etc. For example, the effective amount of microparticles containing an antigen to be delivered to immunize an individual is the amount that results in an immune response sufficient to prevent infection with an organism having the administered antigen.

As used herein, “molecular weight” or “MW” of a polymer or polymeric carrier/scaffold or polymer conjugates refers to the weight average molecular weight unless otherwise specified.

The present disclosure is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C¹³ and C¹⁴.

The present invention is intended to include all isomers of the compound, which refers to and includes, optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers.

### Polymeric Carrier

The conjugates of the present disclosure are useful in biomedical applications, such as drug delivery and tissue engineering, and the polymeric carrier used in the conjugate of the present disclosure is biocompatible and biodegradable. In some embodiments, the polymeric carrier is a soluble polymer, nanoparticle, gel, liposome, micelle, suture, implant, etc.

In some embodiments, the polymeric carrier can have a weight average molecular weight Mw of from about 400 to about 3,000,000 Da, for example, from about 1,000 to about 2,000,000 Da, from about 1,000 to about 1,000,000 Da, from about 1,000 to about 900,000 Da, from about 1,000 to about 800,000 Da, from about 1,000 to about 700,000 Da, from about 1,000 to about 600,000 Da, from about 1,000 to about 500,000 Da, from about 1,000 to about 400,000 Da, from about 1,000 to about 300,000 Da, from about 1,000 to about 200,000 Da, from about 1,000 to about 100,000 Da, from about 1,000 to about 90,000 Da, from about 1,000 to about 80,000 Da, from about 1,000 to about 70,000 Da, from about 1,000 to about 60,000 Da, from about 1,000 to about 50,000 Da, from about 1,000 to about 40,000 Da, from about 1,000 to about 30,000 Da, from about 1,000 to about 20,000 Da, from about 1,000 to about 10,000 Da, from about 2,000 to about 10,000 Da, from about 3,000 to about 10,000 Da, from about 4,000 to about 10,000 Da, from about 5,000 to about 10,000 Da.

In some embodiments, the polymeric carrier used in the present disclosure is polyglycerol. In certain embodiments, the polymeric carrier used in the present disclosure is linear polyglycerol. In certain embodiments, the linear polyglycerol can have a weight average molecular weight Mw of from about 400 to about 3,000,000 Da, for example, from about 1,000 to about 2,000,000 Da, from about 1,000 to about 1,000,000 Da, from about 1,000 to about 900,000 Da, from about 1,000 to about 800,000 Da, from about 1,000 to about 700,000 Da, from about 1,000 to about 600,000 Da, from about 1,000 to about 500,000 Da, from about 1,000 to about 400,000 Da, from about 1,000 to about 300,000 Da, from about 1,000 to about 200,000 Da, from about 1,000 to about 100,000 Da, from about 1,000 to about 90,000 Da, from about 1,000 to about 80,000 Da, from about 1,000 to about 70,000 Da, from about 1,000 to about 60,000 Da, from about 1,000 to about 50,000 Da, from about 1,000 to about 40,000 Da, from about 1,000 to about 30,000 Da, from about 1,000 to about 20,000 Da, from about 1,000 to about 10,000 Da, from about 2,000 to about 10,000 Da, from about 3,000 to about 10,000 Da, from about 4,000 to about 10,000 Da, from about 5,000 to about 10,000 Da.

### Therapeutic Agent

In some embodiments, the therapeutic agent used in the conjugate of the present disclosure is a small molecule having a molecular weight not more than about 5 kDa, not more than about 4 kDa, not more than about 3 kDa, not more than about 1.5 kDa or not more than about 1 kDa.

In some embodiments, the therapeutic agent has an IC₅₀ of less than about 1 nM.

Some therapeutic agents having an IC₅₀ of greater than about 1 nM are unsuitable for conjugation with a targeting moiety using art-recognized conjugation techniques. Without wishing to be bound by theory, such therapeutic agents have a potency that is insufficient for use in targeting moiety-drug conjugates using conventional techniques as sufficient copies of the drug (i.e., more than 8) cannot be conjugated using art-recognized techniques without resulting in diminished pharmacokinetic and physiochemical properties of the conjugate. However, using the conjugation strategies described herein, sufficiently high loadings of these less potent drugs can be achieved, thereby resulting in high loadings of the therapeutic agent while maintaining the desirable pharmacokinetic and physiochemical properties. Therefore, in some embodiment, the therapeutic agent having an IC₅₀ of greater than about 1 nM is useful in the targeting moiety-polymer-drug conjugate provided herein.

The small molecule therapeutic agents used in the present disclosure (e.g., antiproliferative (cytotoxic and cytostatic) agents capable of being linked to a polymer carrier) include cytotoxic compounds (e.g., broad spectrum), angiogenesis inhibitors, cell cycle progression inhibitors, PI3K/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors and RNA polymerase inhibitors.

Broad spectrum cytotoxins include, but are not limited to, DNA-binding, intercalating or alkylating drugs, microtubule stabilizing and destabilizing agents, platinum compounds, topoisomerase I inhibitors and protein synthesis inhibitors.

Exemplary DNA-binding, intercalation or alkylating drugs include, CC-1065 and its analogs, anthracyclines (doxorubicin, epirubicin, idarubicin, daunorubicin, nemorubicin and its derivatives, PNU-159682), bisnapththalimide compounds such as elinafide (LU79553) and its analogs, alkylating agents, such as calicheamicins, dactinomycines, mitromycines, pyrrolobenzodiazepines, and the like. Exemplary CC-1065 analogs include duocarmycin SA, duocarmycin A, duocarmycin C1, duocarmycin C2, duocarmycin B1, duocarmycin B2, duocarmycin D, DU-86, KW-2189, adozelesin, bizelesin, carzelesin, seco-adozelesin, and related analogs and prodrug forms, examples of which are described in U.S. Pat. Nos. 5,475,092; 5,595,499; 5,846,545; 6,534,660; 6,586,618; 6,756,397 and 7,049,316. Doxorubicin and its analogs include those described in U.S. Pat. No. 6,630,579. Calicheamicins include, e.g., enediynes, e.g., esperamicin, and those described in U.S. Pat. Nos. 5,714,586 and 5,739,116. Duocarmycins include those described in U.S. Pat. Nos. 5,070,092; 5,101,038; 5,187,186; 6,548,530; 6,660,742; and 7,553,816 B2; and Li et al., Tet Letts., 50:2932-2935 (2009).

Pyrrolobenzodiazepines (PBD) and analogs thereof include those described in Denny, Exp. Opin. Ther. Patents., 10(4):459-474 (2000) and Antonow and Thurston, Chem Rev., 2815-2864 (2010).

Exemplary microtubule stabilizing and destabilizing agents include taxane compounds, such as paclitaxel, docetaxel, tesetaxel and carbazitaxel, maytansinoids, auristatins and analogs thereof, vinca alkaloid derivatives, epothilones and cryptophycins.

Exemplary maytansinoids or maytansinoid analogs include maytansinol and maytansinol analogs, maytansine or DM-1 and DM-4 are those described in U.S. Pat. Nos. 5,208,020; 5,416,064; 6,333,410; 6,441,163; 6,716,821; RE39,151 and 7,276,497. In certain embodiments, the cytotoxic agent is a maytansinoid, another group of anti-tubulin agents (ImmunoGen, Inc.; see also Chari et al., 1992, Cancer Res. 52:127-131), maytansinoids or maytansinoid analogs. Examples of suitable maytansinoids include maytansinol and maytansinol analogs. Suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Exemplary auristatins include auristatin E (also known as a derivative of dolastatin-10), auristatin EB (AEB), auristatin EFP (AEFP), monomethyl auristatin E (MMAE), monomethyl auristatin F (MMAF), auristatin F, auristatin F phenylenediamine (AFP), auristatin F HPA and dolastatin. Suitable auristatins are also described in U.S. Publication Nos. 2003/0083263, 2011/0020343, and 2011/0070248, PCT Application Publication Nos. WO 09/117531, WO 2005/081711, WO 04/010957, WO 02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, the disclosures of which are incorporated herein by reference in their entirety.

Exemplary vinca alkaloids include vincristine, vinblastine, vindesine, and navelbine (vinorelbine). Suitable Vinca alkaloids that can be used in the present disclosure are also disclosed in U.S. Publication Nos. 2002/0103136 and 2010/0305149, and in U.S. Pat. No. 7,303,749 B1, the disclosures of which are incorporated herein by reference in their entirety.

Exemplary epothilone compounds include epothilone A, B, C, D, E and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO 97/19086; WO 98/08849; WO 98/22461; WO 98/25929; WO 98/38192; WO 99/01124; WO 99/02514; WO 99/03848; WO 99/07692; WO 99/27890; and WO 99/28324; the disclosures of which are incorporated herein by reference in their entirety.

Exemplary cryptophycin compounds are described in U.S. Pat. Nos. 6,680,311 and 6,747,021.

Exemplary platinum compounds include cisplatin (PLATINOL®), carboplatin (PARAPLATIN®), oxaliplatin (ELOXATINE®), iproplatin, ormaplatin, and tetraplatin.

Still other classes of compounds or compounds with these or other cytotoxic modes of action may be selected, including, e.g., mitomycin C, mitomycin A, daunorubicin, doxorubicin, morpholino-doxorubicin, cyanomorpholino-doxorubicin, aminopterin, bleomycin, 1-(chloromethyl)-2,3-dihydro-1H-benzo[e]indol-5-ol, pyrrolobenzodiazepine (PBD) polyamide and dimers thereof. Other suitable cytotoxic agents include, for example, puromycins, topotecan, rhizoxin, echinomycin, combretastatin, netropsin, estramustine, cryptophysins, cemadotin, discodermolide, eleutherobin, and mitoxantrone.

Exemplary topoisomerase I inhibitors include camptothecin, camptothecin derivatives, camptothecin analogs and non-natural camptothecins, such as, for example, CPT-11 (irinotecan), SN-38, GI-147211C, topotecan, 9-aminocamptothecin, 7-hydroxymethyl camptothecin, 7-aminomethyl camptothecin, 10-hydroxycamptothecin, (20S)-camptothecin, rubitecan, gimatecan, karenitecin, silatecan, lurtotecan, exatecan, diflomotecan, belotecan, lurtotecan and 539625. Other camptothecin compounds that can be used in the present invention include those described in, for example, J. Med. Chem., 29:2358-2363 (1986); J. Med. Chem., 23:554 (1980); J. Med. Chem., 30:1774 (1987).

Angiogenesis inhibitors include, but are not limited, MetAP2 inhibitors, VEGF inhibitors, PIGF inhibitors, VGFR inhibitors, PDGFR inhibitors, MetAP2 inhibitors. Exemplary VGFR and PDGFR inhibitors include sorafenib (Nexavar), sunitinib (Sutent) and vatalanib. Exemplary MetAP2 inhibitors include fumagillol analogs, meaning any compound that includes the fumagillin core structure, including fumagillamine, that inhibits the ability of MetAP-2 to remove NH₂-terminal methionines from proteins as described in Rodeschini et al., J. Org. Chem., 69, 357-373, 2004 and Liu, et al., Science 282, 1324-1327, 1998. Non limiting examples of “fumagillol analogs” are disclosed in J. Org. Chem., 69, 357, 2004; J. Org. Chem., 70, 6870, 2005; European Patent Application 0 354 787; J. Med. Chem., 49, 5645, 2006; Bioorg. Med. Chem., 11, 5051, 2003; Bioorg. Med. Chem., 14, 91, 2004; Tet. Lett. 40, 4797, 1999; WO99/61432; U.S. Pat. Nos. 6,603,812; 5,789,405; 5,767,293; 6,566,541; and 6,207,704.

Exemplary cell cycle progression inhibitors include CDK inhibitors such as, for example, BMS-387032 and PD0332991; Rho-kinase inhibitors such as, for example GSK429286; checkpoint kinase inhibitors such as, for example, AZD7762; aurora kinase inhibitors such as, for example, AZD1152, MLN8054 and MLN8237; PLK inhibitors such as, for example, BI 2536, B16727 (Volasertib), GSK461364, ON-01910 (Estybon); and KSP inhibitors such as, for example, SB 743921, SB 715992 (ispinesib), MK-0731, AZD8477, AZ3146 and ARRY-520.

Exemplary PI3K/m-TOR/AKT signaling pathway inhibitors include phosphoinositide 3-kinase (PI3K) inhibitors, GSK-3 inhibitors, ATM inhibitors, DNA-PK inhibitors and PDK-1 inhibitors.

Exemplary PI3 kinase inhibitors are disclosed in U.S. Pat. No. 6,608,053, and include BEZ235, BGT226, BKM120, CAL101, CAL263, demethoxyviridin, GDC-0941, GSK615, IC87114, LY294002, Palomid 529, perifosine, PI-103, PF-04691502, PX-866, SAR245408, SAR245409, SF1126, Wortmannin, XL147 and XL765.

Exemplary AKT inhibitors include, but are not limited to AT7867.

Exemplary MAPK signaling pathway inhibitors include MEK, Ras, JNK, B-Raf and p38 MAPK inhibitors.

Exemplary MEK inhibitors are disclosed in U.S. Pat. No. 7,517,994 and include GDC-0973, GSK1120212, MSC1936369B, AS703026, R05126766 and R04987655, PD0325901, AZD6244, AZD 8330 and GDC-0973.

Exemplary B-raf inhibitors include CDC-0879, PLX-4032, and SB590885.

Exemplary B p38 MAPK inhibitors include BIRB 796, LY2228820 and SB 202190.

Receptor tyrosine kinases (RTK) are cell surface receptors which are often associated with signaling pathways stimulating uncontrolled proliferation of cancer cells and neoangiogenesis. Many RTKs, which over express or have mutations leading to constitutive activation of the receptor, have been identified, including, but not limited to, VEGFR, EGFR, FGFR, PDGFR, EphR and RET receptor family receptors. Exemplary specific RTK targets include ErbB2, FLT-3, c-Kit, and c-Met.

Exemplary inhibitors of ErbB2 receptor (EGFR family) include but not limited to AEE788 (NVP-AEE 788), BIBW2992, (Afatinib), Lapatinib, Erlotinib (Tarceva), and Gefitinib (Iressa).

Exemplary RTK inhibitors targeting more then one signaling pathway (multitargeted kinase inhibitors) include AP24534 (Ponatinib) that targets FGFR, FLT-3, VEGFR-PDGFR and Bcr-Abl receptors; ABT-869 (Linifanib) that targets FLT-3 and VEGFR-PDGFR receptors; AZD2171 that targets VEGFR-PDGFR, Flt-1 and VEGF receptors; CHR-258 (Dovitinib) that targets VEGFR-PDGFR, FGFR, Flt-3, and c-Kit receptors; Sunitinib (Sutent) that targets VEGFR, PDGFR, KIT, FLT-3 and CSF-IR; Sorafenib (Nexavar) and Vatalanib that target VEGFR, PDGFR as well as intracellular serine/threonine kinases in the Raf/Mek/Erk pathway.

Exemplary protein chaperon inhibitors include HSP90 inhibitors. Exemplary HSP90 inhibitors include 17AAG derivatives, BIIB021, BIIB028, SNX-5422, NVP-AUY-922 and KW-2478.

Exemplary HDAC inhibitors include Belinostat (PXD101), CUDC-101, Droxinostat, ITF2357 (Givinostat, Gavinostat), JNJ-26481585, LAQ824 (NVP-LAQ824, Dacinostat), LBH-589 (Panobinostat), MC1568, MGCD0103 (Mocetinostat), MS-275 (Entinostat), PCI-24781, Pyroxamide (NSC 696085), SB939, Trichostatin A and Vorinostat (SAHA).

Exemplary PARP inhibitors include iniparib (BSI 201), olaparib (AZD-2281), ABT-888 (Veliparib), AG014699, CEP 9722, MK 4827, KU-0059436 (AZD2281), LT-673,3-aminobenzamide, A-966492, and AZD2461.

Exemplary Wnt/Hedgehog signaling pathway inhibitors include vismodegib (RG3616/GDC-0449), cyclopamine (11-deoxojervine) (Hedgehog pathway inhibitors) and XAV-939 (Wnt pathway inhibitor).

Exemplary RNA polymerase inhibitors include amatoxins. Exemplary amatoxins include α-amanitins, β-amanitins, γ-amanitins, ε-amanitins, amanullin, amanullic acid, amaninamide, amanin, and proamanullin.

Exemplary protein synthesis inhibitors include trichothecene compounds.

In some embodiment, the therapeutic agent of the present disclosure is a topoisomerase inhibitor (such as, for example, a non-natural camptothecin compound), vinca alkaloid, kinase inhibitor (e.g., PI3 kinase inhibitor (GDC-0941 and PI-103)), MEK inhibitor, KSP inhibitor, RNA polymerase inhibitor, protein synthesis inhibitor, PARP inhibitor, docetaxel, paclitaxel, doxorubicin, duocarmycin, auristatin, dolastatin, calicheamicins, topotecan, SN38, camptothecin, exatecan, nemorubicin and its derivatives, PNU-159682, CC1065, elinafide, trichothecene, pyrrolobenzodiazepines, maytansinoids, DNA-binding drugs or a platinum compound, and analogs thereof. In some embodiments, the therapeutic agent is a derivative of SN-38, camptothecin, topotecan, exatecan, calicheamicin, exatecan, nemorubicin, PNU-159682, anthracycline, maytansinoid, taxane, trichothecene, CC1065, elinafide, vindesine, vinblastine, PI-103, AZD 8330, dolastatin, auristatin E, auristatin F, a duocarmycin compound, ispinesib, pyrrolobenzodiazepine, ARRY-520 and stereoisomers, isosteres and analogs thereof.

In some embodiments, the therapeutic agent used in the present disclosure is a combination of two or more drugs, such as, for example, PI3 kinase inhibitors and MEK inhibitors; broad spectrum cytotoxic compounds and platinum compounds; PARP inhibitors and platinum compounds; broad spectrum cytotoxic compounds and PARP inhibitors.

In yet another embodiment, the therapeutic agent used in the present disclosure is auristatin F-hydroxypropylamide-L-alanine.

One skilled in the art will readily understand that each of the therapeutic agents described herein can be modified in such a manner that the resulting compound still retains the specificity and/or activity of the original compound. The skilled artisan will also understand that many of these compounds can be used in place of the therapeutic agents described herein. Thus, the therapeutic agents of the present disclosure include analogs and derivatives of the compounds described herein.

In some embodiments, the therapeutic agent has antiproliferative activity against a target cell or pathway.

In certain embodiments, the antiproliferative activity is selected from cytostatic and/or cytotoxic activity.

In certain embodiments, the therapeutic agent is selected from anti-cancer substances, cytotoxic drugs, radionuclides, vitamins, anti-AIDS substances, antibiotics, immunosuppressants, immunomodulatory compounds, therapeutic RNAs, anti-viral substances, enzyme inhibitors, neurotoxins, opioids, hypnotics, anti-histamines, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, antisecretory factors, anticoagulants and/or antithrombotic agents, local anesthetics, ophthalmics, prostaglandins, anti-depressants, anti-psychotic substances, anti-emetics, imaging agents.

In certain embodiments, the therapeutic agent comprises amino acid-based molecules.

In certain embodiments, the amino acid-based molecules comprise peptides, polypeptides, enzymes, antibodies, immunoglobulins, or functional fragments thereof.

In certain embodiments, the therapeutic agent has a chemically reactive group.

In certain embodiments, the chemically reactive group comprises —COOH, primary amine, secondary amine-NHR, —OH, —SH, —C(O)H, C(O)R¹⁴. —C(O)NHR¹⁵, C(S)OH, —S(O)₂OR¹⁵, —P(O)₂OR¹⁵, —CN, —NC or —ONO, in which R¹⁴ is selected from aliphatic, heteroaliphatic, carbocyclic or heterocycloalkyl moiety and R¹⁵ is selected from a hydrogen, aliphatic, heteroaliphatic, cycloalkyl, heterocycloalkyl, aryl or heteroaryl.

### Linker-Polymer-Drug Compound

In one aspect, there is provided a polymeric scaffold of Formula (I) useful to conjugate with a targeting moiety:

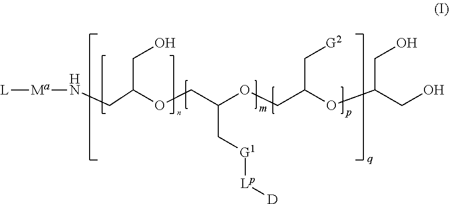

wherein:
the polymeric scaffold comprises linear polyglycerol;
L is a linking moiety comprising a functional group Wₚ that is capable of forming a covalent bond with the targeting moiety;
M^{a} is a stretcher connecting L to —NH— moiety;
each G¹ is independently a functional group connecting L^{p} to the linear polyglycerol;
each L^{p} is independently a drug release mechanism between a therapeutic agent moiety D and G¹;
each D is independently a therapeutic agent moiety;
each G² is independently a functional group capable of converting into a charged state;
n is an integer from 0 to 1000;
m is an integer from 0 to 1000;
p is an integer from 0 to 1000; and
q is an integer from 0 to 1000.

In some embodiments, Wₚ is capable of reacting with a functional group on the targeting moiety with a click reaction.

In certain embodiments, Wₚ is selected from the group consisting of:

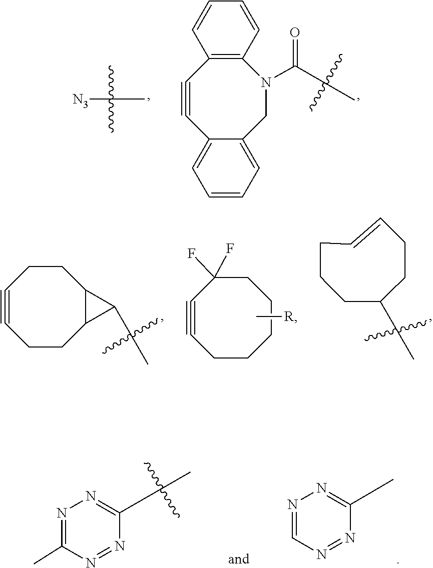

In some embodiments, Wₚ is capable of reacting with amino acids on the targeting moiety.

In certain embodiments, Wₚ is capable of reacting with amino acids on the targeting moiety, and the amino acids are natural amino acids, non-natural amino acids or combination thereof. In certain embodiments, the natural amino acid may comprise cysteine, lysine, tyrosine, aspartic acid and glutamic acid.

In some embodiments, Wₚ is capable of reacting with one or more cysteines on the targeting moiety.

In certain embodiments, Wₚ is capable of reacting with one or more cysteines on the targeting moiety and each Wₚ is selected from the group consisting of:

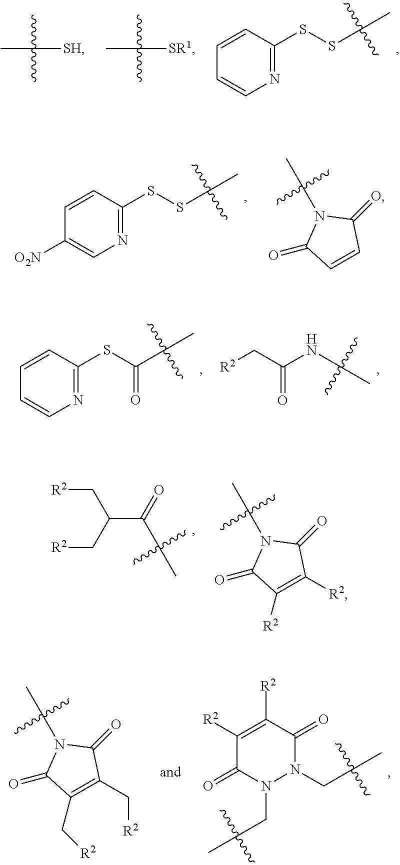

wherein R¹ is a sulfur protecting group, and each R² is independently a leaving group.

In certain embodiments, each R² is independently selected from halo or R^{2a}C(O)O—, in which R^{2a} is hydrogen, aliphatic, heteroaliphatic, cycloalkyl, heterocycloalkyl, aryl or heteroaryl.

In some embodiments, Wₚ is capable of reacting with one or more lysines on the targeting moiety.

In certain embodiments, Wₚ is capable of reacting with one or more lysines on the targeting moiety, and each Wₚ is independently selected from the group consisting of:

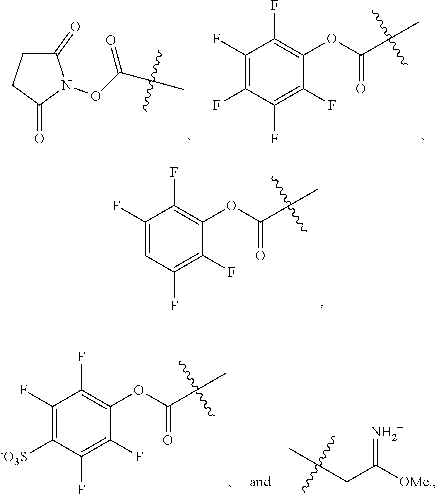

In some embodiments, Wₚ is capable of reacting with one or more non-natural amino acids on the targeting moiety.

In certain embodiments, Wₚ is capable of reacting with one or more non-natural amino acids on the targeting moiety and each Wₚ is independently selected from:

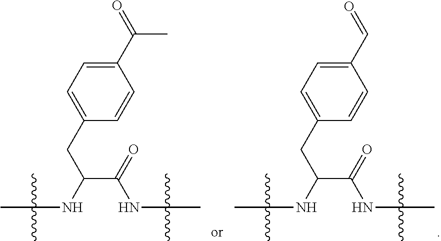 [SMILES: *NC(=O)C(Cc1ccc(C(C)=O)cc1)N* ; *NC(=O)C(Cc1ccc(C=O)cc1)N*]

In some embodiments, M^{a} is selected from the group consisting of:

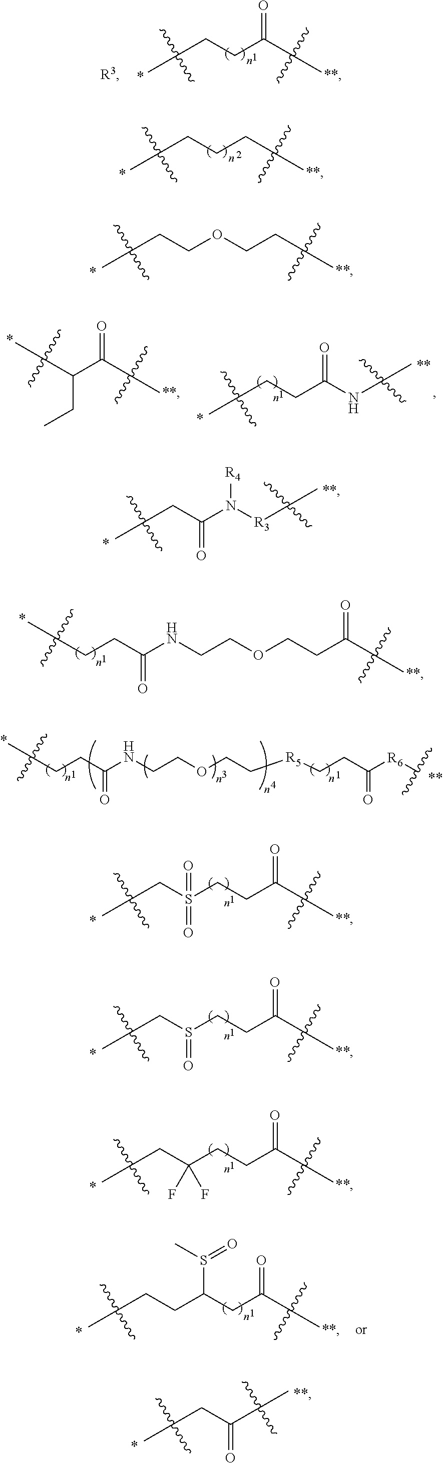

wherein
   * is the site covalently attached to L, ** is the site covalently attached to —NH-moiety;
   R³ is C₁₋₁₀ alkyl, C₁₋₁₀ heteroalkyl, C₃₋₈ cycloalkyl, —O—(C₁₋₈ alkyl)-, aryl, —C₁₋₁₀ alkyl-aryl-, -aryl-C₁₋₁₀ alkyl-, —C₁₋₁₀ alkyl-(C₃₋₈ cycloalkyl)-, —(C₃₋₈ cycloalkyl-C₁₋₁₀ alkyl)-, 4- to 14-membered heterocycloalkyl, —C₁₋₁₀ alkyl-(4- to 14-membered heterocycloalkyl)-, -(4- to 14-membered heterocycloalkyl)-C₁₋₁₀ alkyl-, —C₁₋₁₀ alkyl-C(═O)—, —C₁₋₁₀ heteroalkyl-C(═O)—, —C₃₋₈ cycloalkyl-C(═O)—, —O—(C₁₋₈ alkyl)-C(═O)—, -aryl-C(═O)—, —C₁₋₁₀ alkyl-aryl-C(═O)—, -aryl-C₁₋₁₀ alkyl-C(═O)—, —C₁₋₁₀ alkyl-(C₃₋₈ cycloalkyl)-C(═O)—, —(C₃₋₈ cycloalkyl)-C₁₋₁₀ alkyl-C(═O)—, -4- to 14-membered heterocycloalkyl-C═(O)—, —C₁₋₁₀ alkyl-(4- to 14-membered heterocycloalkyl)-C(═O)—, -(4- to 14-membered heterocycloalkyl)-C₁₋₁₀ alkyl-C(═O)—, —C₁₋₁₀ alkyl-NH—, —C₁₋₁₀ heteroalkyl-NH—, —C₃₋₈ cycloalkyl-NH—, —O—(C₁₋₈ alkyl)-NH—, -aryl-NH—, —C₁₋₁₀ alkyl-aryl-NH—, -aryl-C₁₋₁₀ alkyl-NH—, —C₁₋₁₀ alkyl-(C₃₋₈ cycloalkyl)-NH—, —(C₃₋₈ cycloalkyl)-C₁₋₁₀ alkyl-NH—, -4- to 14-membered heterocycloalkyl-NH—, —C₁₋₁₀ alkyl-(4- to 14-membered heterocycloalkyl)-NH—, -(4- to 14-membered heterocycloalkyl)-C₁₋₁₀ alkyl-NH—, —C₁₋₁₀ alkyl-S—, —C₁₋₁₀ heteroalkyl-S—, —C₃₋₈ cycloalkyl-S—, —O—C₁₋₈ alkyl-S—, -aryl-S—, —C₁₋₁₀ alkyl-aryl-S—, -aryl-C₁₋₁₀ alkyl-S—, —C₁₋₁₀ alkyl-(C₃₋₈ cycloalkyl)-S—, —(C₃₋₈ cycloalkyl)-C₁₋₁₀ alkyl-S—, -4- to 14-membered heterocycloalkyl-S—, —C₁₋₁₀ alkyl-(4- to 14-membered heterocycloalkyl)-S—, or -(4- to 14-membered heterocycloalkyl)-C₁₋₁₀ alkyl-S—;
   each R⁴ independently is hydrogen, C₁₋₆ alkyl, C₆₋₁₀ aryl, C₃₋₈ cycloalkyl, —COOH or —COO—C₁₋₆ alkyl;
   R⁵ is —C(O)—NR^{5a} or —NR^{5a}—C(O)—;
   R^{5a} is hydrogen, C₁₋₆ alkyl, C₆₋₁₀ aryl, C₃₋₈ cycloalkyl, —COOH or —COO—C₁₋₆ alkyl;
   R⁶ is a bond or —NR^{6a}—(CR^{6b}R^{6c})—C(O)—;
   R^{6a} is hydrogen, C₁₋₆ alkyl, C₆₋₁₀ aryl, C₃₋₈ cycloalkyl, —COOH or —COO—C₁₋₆ alkyl;
   each R^{6b} and R^{6c} independently is hydrogen, C₁₋₆ alkyl, C₆₋₁₀ aryl, hydroxylated C₆₋₁₀ aryl, polyhydroxylated C₆₋₁₀ aryl, 5- to 12-membered heterocycloalkyl, C₃₋₈ cycloalkyl, hydroxylated C₃₋₈ cycloalkyl, polyhydroxylated C₃₋₈ cycloalkyl or a side chain of a natural or unnatural amino acid;
   each n¹ independently is an integer from 0 to 6;
   n² is an integer from 0 to 8;
   each n³ independently is an integer from 1 to 6; and
   n⁴ is an integer from 1 to 4.

In certain embodiments, M^{a} is selected from the group consisting of:

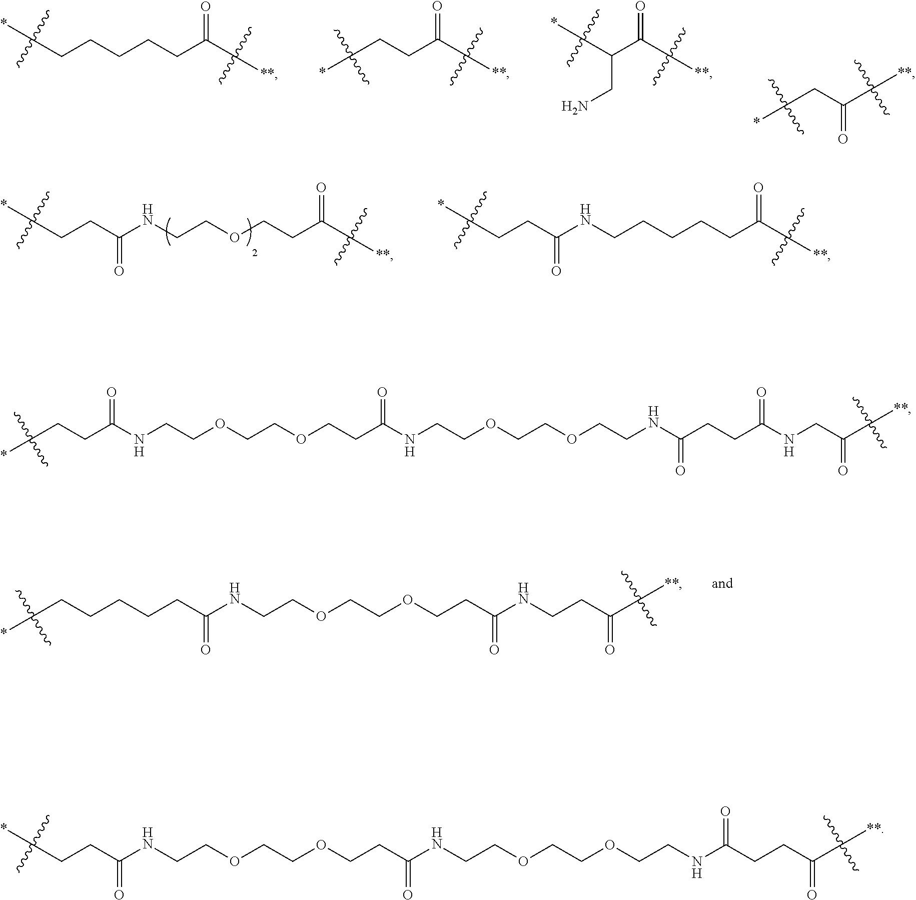

In some embodiments, G¹ is selected from the group consisting of:

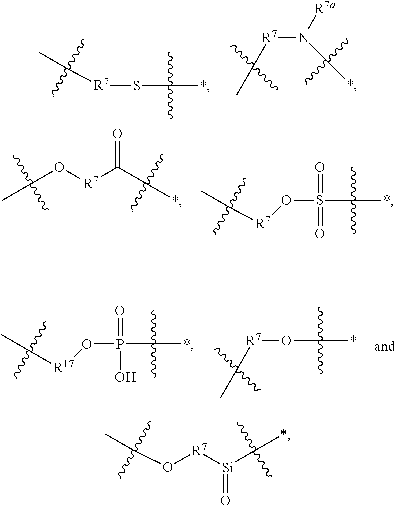

wherein * is the site covalently attached to L^{p}, each R⁷ is independently selected from a direct bond, alkyl, cycloalkyl, heterocycloalkyl, aryl, or heteroaryl, and R^{7a} is selected from hydrogen, an aliphatic, heteroaliphatic, cycloalkyl, or heterocycloalkyl moiety.

In some embodiments, G¹ is selected from the group consisting of:

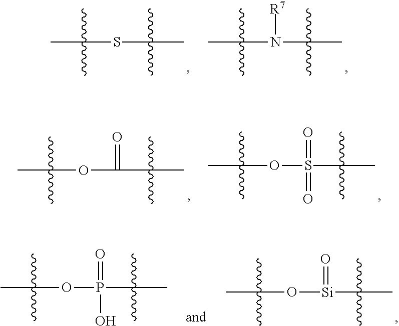

wherein R⁷ is selected from hydrogen, aliphatic, heteroaliphatic, cycloalkyl, heterocycloalkyl, aryl or heteroaryl. In some embodiments, each L^{p} independently comprises a labile structure.

In certain embodiments, the labile structure is selected from hydrolytically labile structures or enzymatic labile structures.

In certain embodiments, the hydrolytically labile structure is selected from the group consisting of:

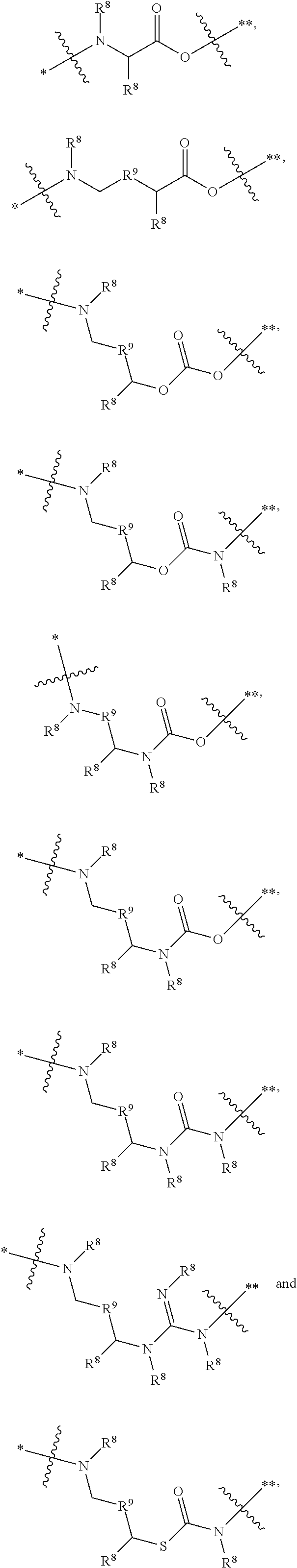

wherein * is the site covalently attached to G¹, ** is the site covalently attached to D, R⁸ is selected from hydrogen, alky or aryl, R9 is selected from aliphatic, heteroaliphatic, cycloalkyl, heterocycloalkyl, aryl, or heteroaryl.

In certain embodiments, the hydrolytically labile structure is selected from the group consisting of:

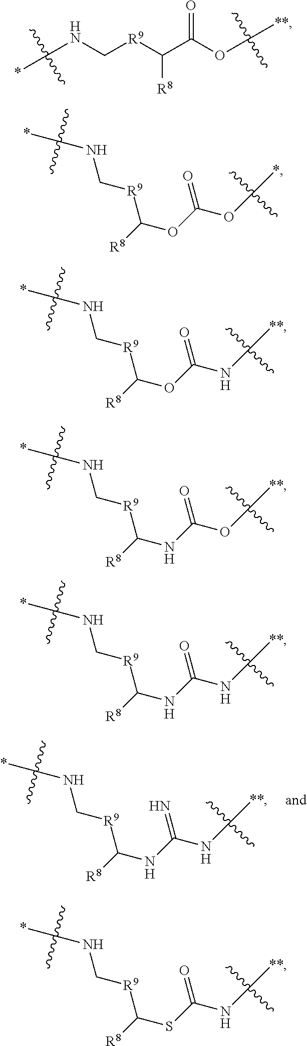

wherein * is the site covalently attached to G¹, ** is the site covalently attached to D, R⁸ is selected from hydrogen, alky or aryl, R⁹ is selected from aliphatic, heteroaliphatic, cycloalkyl, heterocycloalkyl, aryl, or heteroaryl.

In some embodiments, G¹ is

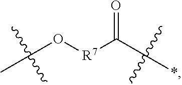

wherein * is the site covalently attached to L^{p}, R⁷ is alkyl.

In certain embodiments, -G¹-L^{p}-D is

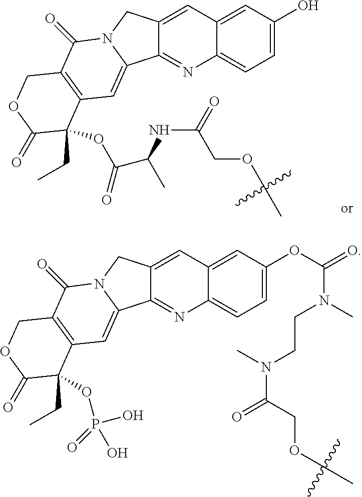 [SMILES: *OCC(=O)N[C@@H](C)C(=O)O[C@]1(CC)C(=O)OCc2c1cc1n(c2=O)Cc2cc3cc(O)ccc3nc2-1 ; *OCC(=O)N(C)CCN(C)C(=O)Oc1ccc2nc3c(cc2c1)Cn1c-3cc2c(c1=O)COC(=O)[C@@]2(CC)OP(=O)(O)O]

In some embodiments, the enzymatic labile structure is liable to enzymes selected from Cathepsin B, phosphatase, sulfatase, or glucuronidase.

In certain embodiments, the enzymatic labile structure is liable to cathepsin B and is selected from —Z— or

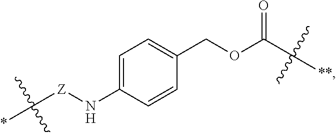

wherein Z is a substrate for cathepsin B comprising 2 to 4 amino acids.

In some embodiments, G¹ is

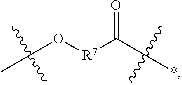

wherein * is the site covalently attached to L^{p}, R⁷ is alkyl.

In certain embodiments, -G¹-L^{p}-D is

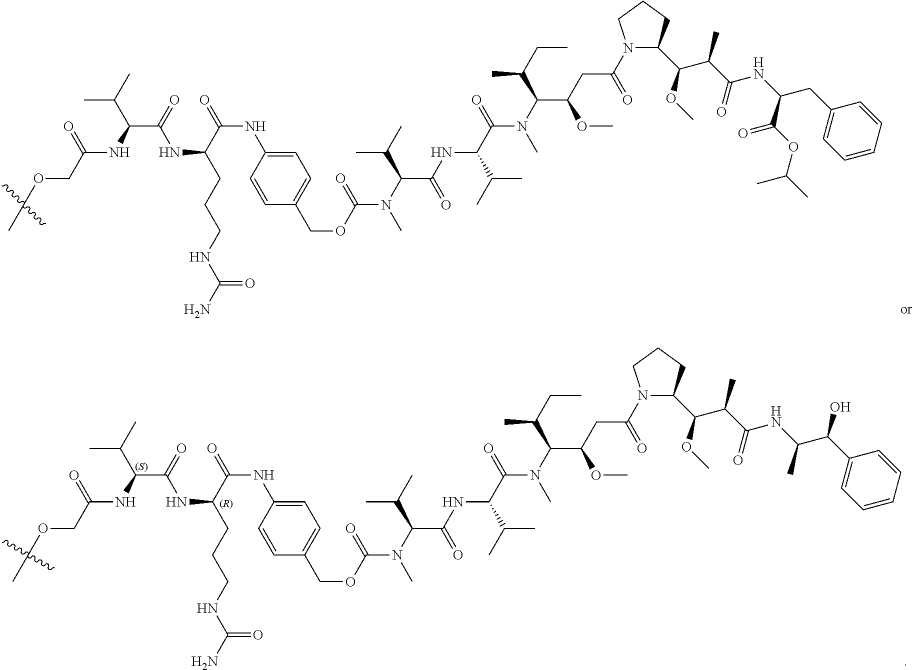

In certain embodiments, the enzymatic labile structure is liable to glucuronidase and is

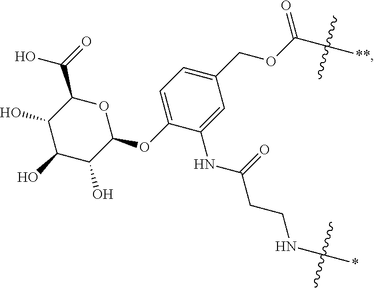

wherein * is the site covalently attached to G¹, ** is the site covalently attached to D.

In certain embodiments, G¹ is

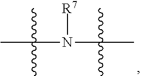

wherein R⁷ is selected from hydrogen, aliphatic, heteroaliphatic, cycloalkyl, or heterocycloalkyl.

In some embodiments, G¹ is

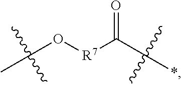

wherein * is the site covalently attached to L^{p}, R⁷ is alkyl.

In certain embodiments, -L^{p}-D is selected from:

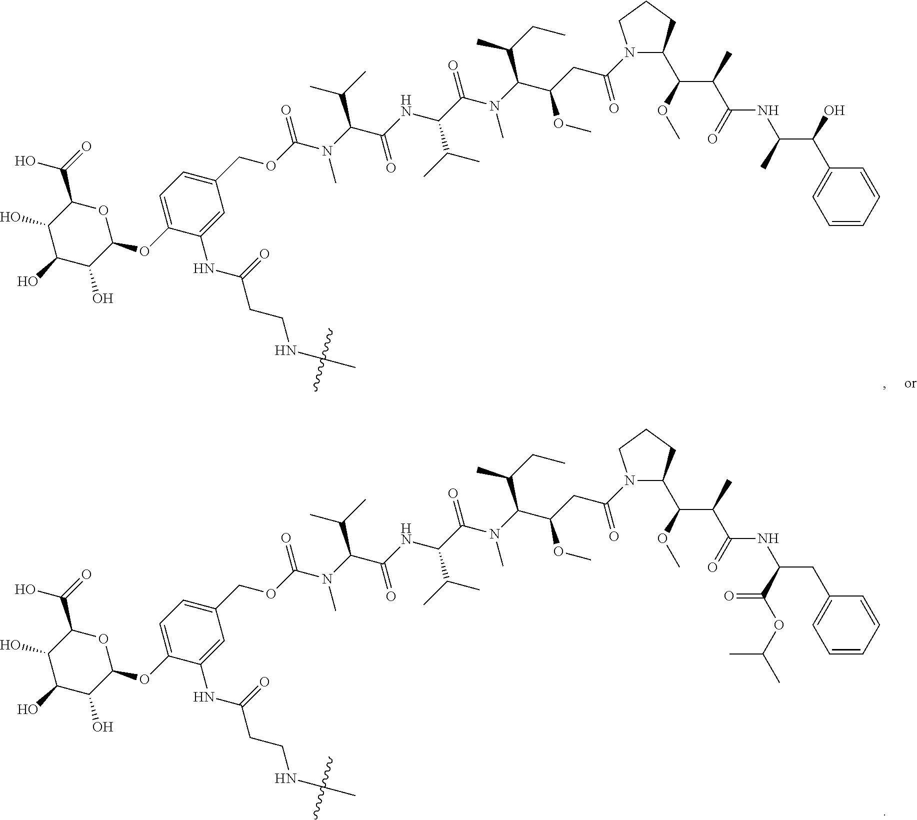 [SMILES: *NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)ccc1O[C@@H]1O[C@H](C(=O)O)[C@@H](O)[C@H](O)[C@H]1O ; *NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)C(=O)OC(C)C)OC)C(C)C)C(C)C)ccc1O[C@@H]1O[C@H](C(=O)O)[C@@H](O)[C@H](O)[C@H]1O]

In certain embodiments, -G¹-L^{p}-D is selected from:

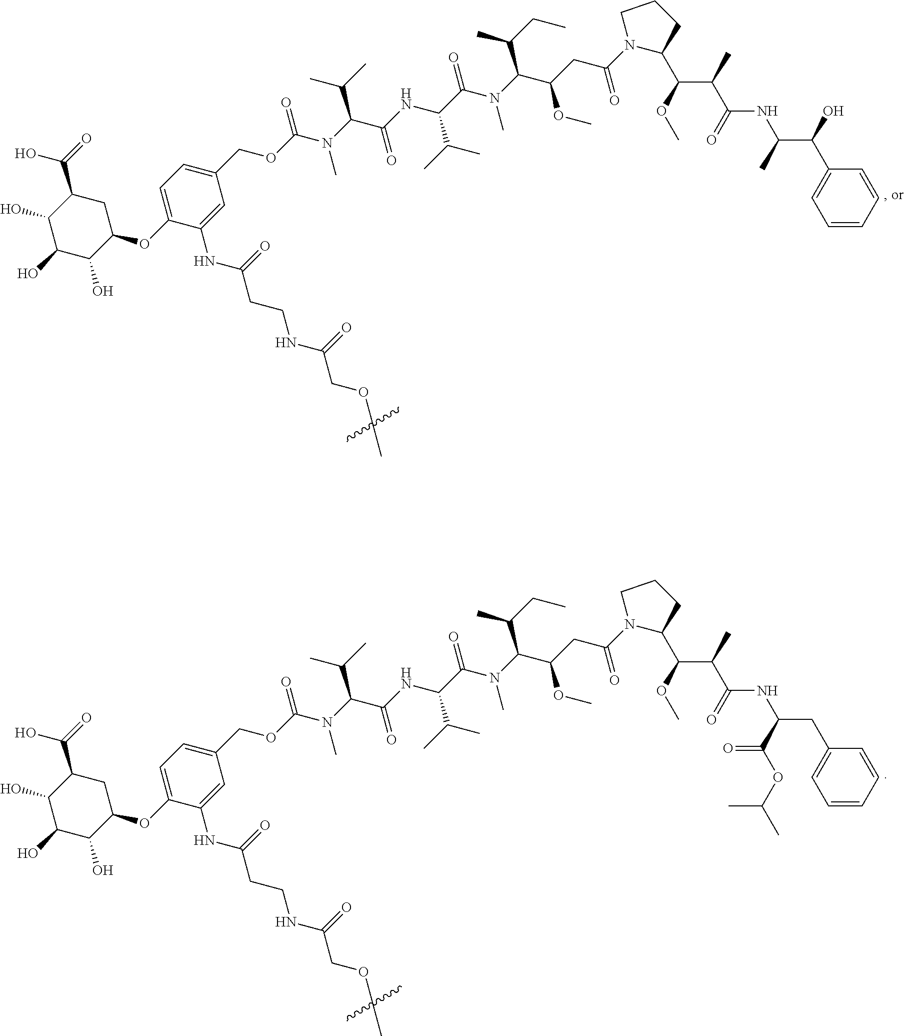 [SMILES: *OCC(=O)NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)ccc1O[C@@H]1C[C@H](C(=O)O)[C@@H](O)[C@H](O)[C@H]1O ; *OCC(=O)NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)C(=O)OC(C)C)OC)C(C)C)C(C)C)ccc1O[C@@H]1C[C@H](C(=O)O)[C@@H](O)[C@H](O)[C@H]1O]

In certain embodiments, the enzymatic labile structure is liable to phosphatase and is selected from

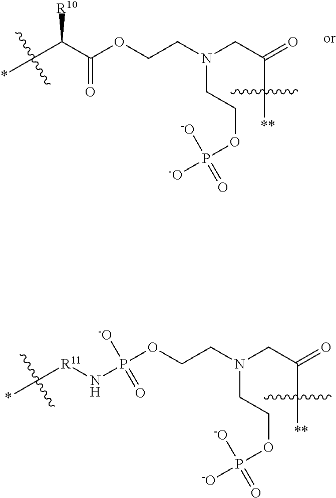

wherein * is the site covalently attached to G¹, ** is the site covalently attached to D, each of R¹⁰ and R¹¹ is independently hydrogen, aliphatic, heteroaliphatic, cycloalkyl, or heterocycloalkyl moiety.

In certain embodiments, G¹ is

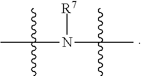

In certain embodiments, -G¹-L^{p}-D is selected from the group consisting of:

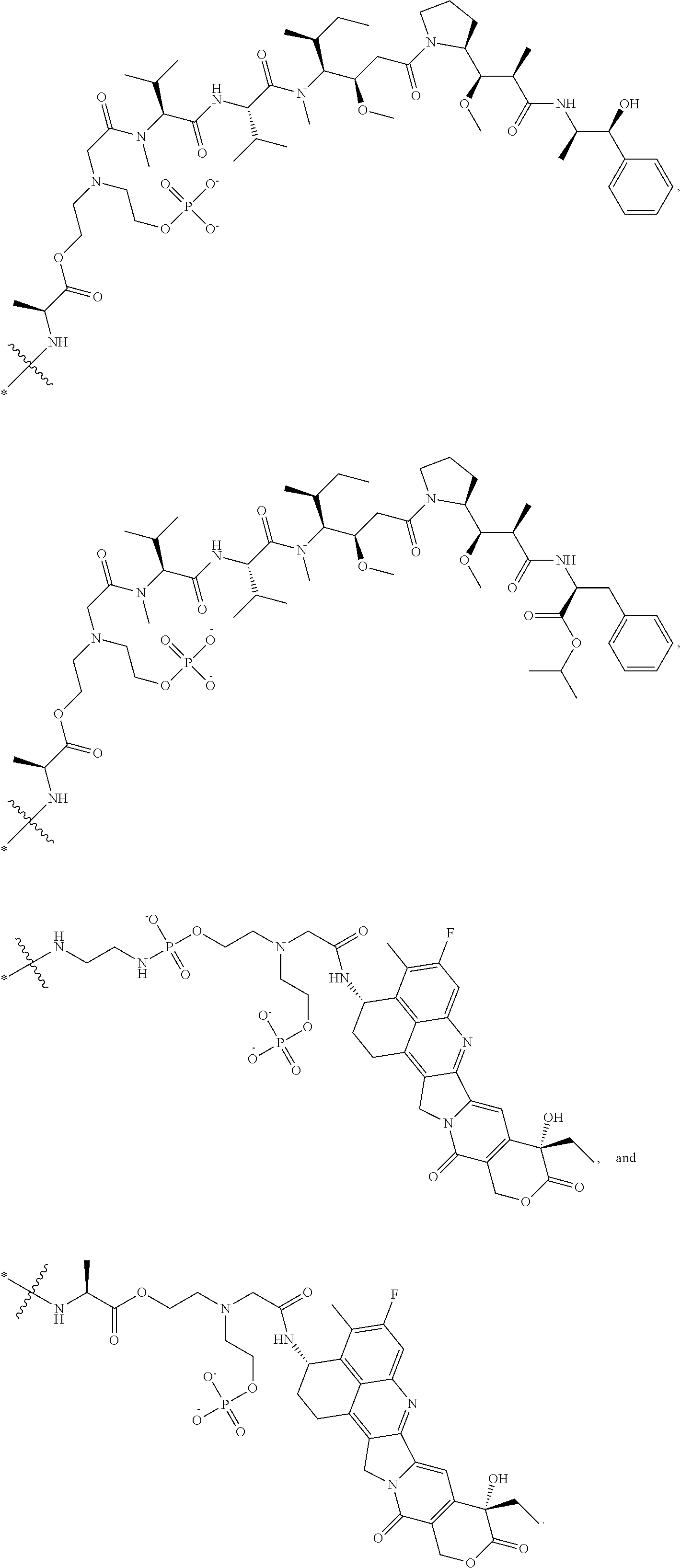

In certain embodiments, the enzymatic labile structures are liable to sulfatase and is

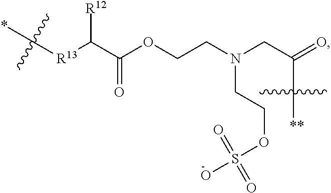

wherein * is the site covalently attached to G¹, ** is the site covalently attached to D, each of R¹² and R¹³ is independently hydrogen, aliphatic, heteroaliphatic, cycloalkyl, or heterocycloalkyl moiety.

In some embodiments, G¹ is

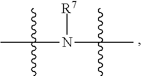

wherein R⁷ is selected from hydrogen, aliphatic, heteroaliphatic, cycloalkyl, or heterocycloalkyl moiety.

In some embodiments, G¹ is

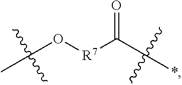

wherein * is the site covalently attached to L^{p}, R⁷ is alkyl.

In certain embodiments, -G¹-L^{p}-D is:

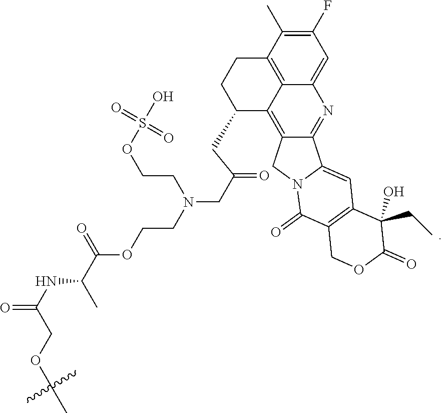 [SMILES: *OCC(=O)N[C@@H](C)C(=O)OCCN(CCOS(=O)(=O)O)CC(=O)C[C@@H]1CCc2c(C)c(F)cc3nc4c(c1c23)Cn1c-4cc2c(c1=O)COC(=O)[C@]2(O)CC]

In some embodiments, n is an integer from 1 to 100; m is an integer from 1 to 100; and p is an integer from 1 to 50.

In some embodiments, the therapeutic agent has antiproliferative activity against a target cell or pathway.

In certain embodiments, the antiproliferative activity is selected from cytostatic and/or cytotoxic activity.

In certain embodiments, the therapeutic agent is selected from anti-cancer substances, cytotoxic drugs, radionuclides, vitamins, anti-AIDS substances, antibiotics, immunosuppressants, immunomodulatory compounds, therapeutic RNAs, anti-viral substances, enzyme inhibitors, neurotoxins, opioids, hypnotics, anti-histamines, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, antisecretory factors, anticoagulants and/or antithrombotic agents, local anesthetics, ophthalmics, prostaglandins, anti-depressants, anti -psychotic substances, anti-emetics, imaging agents.

In certain embodiments, the therapeutic agent comprises amino acid-based molecules.

In certain embodiments, the amino acid-based molecules comprise peptides, polypeptides, enzymes, antibodies, immunoglobulins, or functional fragments thereof.

In certain embodiments, the therapeutic agent has a chemically reactive group.

In certain embodiments, the chemically reactive group comprises —COOH, primary amine, secondary amine-NHR, —OH, —SH, —C(O)H, C(O)R¹⁴. —C(O)NHR¹⁵, C(S)OH, —S(O)₂OR¹⁵, —P(O)₂OR¹⁵, —CN, —NC or —ONO, in which R¹⁴ is selected from aliphatic, heteroaliphatic, cycloalkyl, heterocycloalkyl, aryl or heteroaryl, and R¹⁵ is selected from a hydrogen, aliphatic, heteroaliphatic, cycloalkyl, heterocycloalkyl, aryl or heteroaryl.

In certain embodiments, G² is selected from the group consisting of:

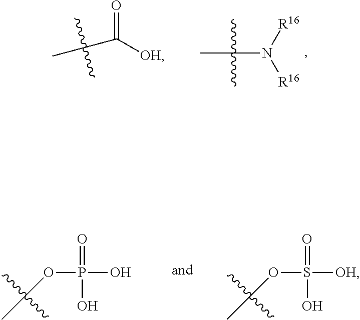

wherein each R¹⁶ is independently hydrogen, aliphatic, heteroaliphatic, cycloalkyl, heterocycloalkyl, aryl or heteroaryl.

In certain embodiments, G² is

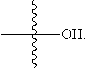 [SMILES: *O]

### Linker-Polymer Compound

The present disclosure also relates to a linker-polymer compound that can further bind to a drug moiety to form the linker-polymer-drug compounds provided herein.

Therefore, in a further aspect, the present disclosure provides a polymeric scaffold of Formula (II):

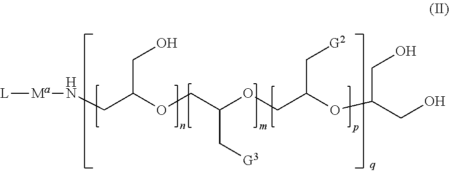

wherein,
   the polymeric scaffold comprises linear polyglycerol;
   L is a linking moiety comprising a functional group Wₚ that is capable of forming a covalent bond with the targeting moiety;
   M^{a} is a stretcher connecting L to —NH— moiety;
   each G² is independently a functional group capable of converting into a charged state;
   each G³ independently comprises a functional group capable of reacting with a reactive group in a drug release mechanism to connect the drug release mechanism to the linear polyglycerol;
   n is an integer from 0 to 1000;
   m is an integer from 0 to 1000;
   p is an integer from 0 to 1000; and
   q is an integer from 0 to 1000.

In some embodiments, n is 2; m is 2; and p is 2.

In some embodiments, q is an integer from 3 to 5.

In some embodiments, Wₚ is capable of reacting with a functional group on the targeting moiety with a click reaction.

In certain embodiments, Wₚ is selected from the group consisting of:

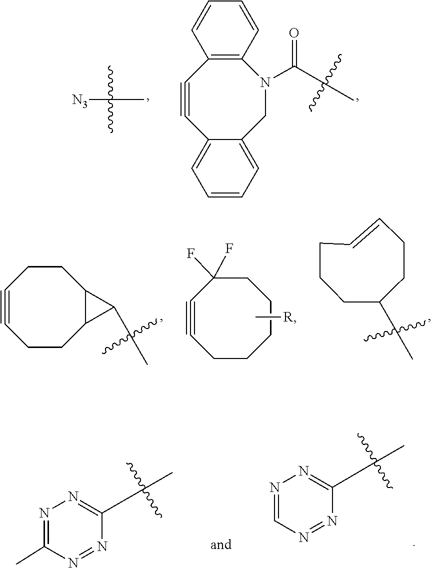

In some embodiments, Wₚ is capable of reacting with amino acids on the targeting moiety.

In certain embodiments, Wₚ is capable of reacting with amino acids on the targeting moiety, and the amino acids are natural amino acids, non-natural amino acids or combination thereof. In certain embodiments, the natural amino acid may comprise cysteine, lysine, tyrosine, aspartic acid and glutamic acid.

In some embodiments, Wₚ is capable of reacting with one or more cysteines on the targeting moiety.

In certain embodiments, Wₚ is capable of reacting with one or more cysteines on the targeting moiety and each Wₚ is selected from the group consisting of:

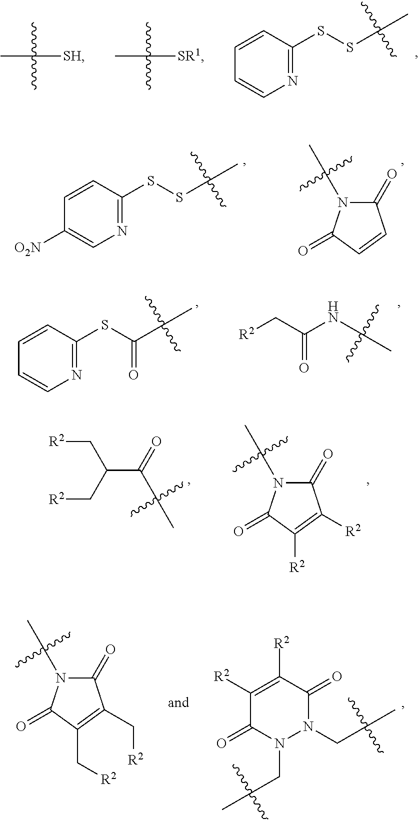

wherein R¹ is a sulfur protecting group, and each R² is independently a leaving group.

In certain embodiments, each R² is independently selected from halo or R^{2a}C(O)O—, in which R^{2a} is hydrogen, aliphatic, heteroaliphatic, cycloalkyl, heterocycloalkyl, aryl or heteroaryl.

In some embodiments, Wₚ is capable of reacting with one or more lysines on the targeting moiety.

In certain embodiments, Wₚ is capable of reacting with one or more lysines on the targeting moiety, and each Wₚ is independently selected from the group consisting of:

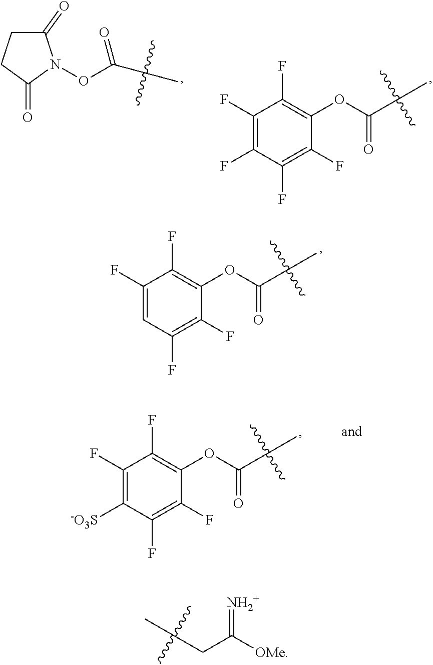

In some embodiments, Wₚ is capable of reacting with one or more non-natural amino acids on the targeting moiety.

In certain embodiments, Wₚ is capable of reacting with one or more non-natural amino acids on the targeting moiety and each Wₚ is independently selected from:

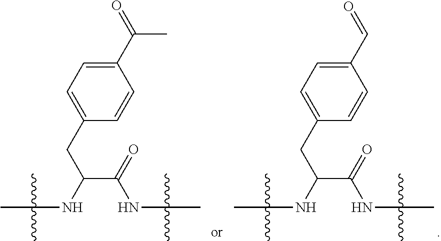 [SMILES: *NC(=O)C(Cc1ccc(C(C)=O)cc1)N* ; *NC(=O)C(Cc1ccc(C=O)cc1)N*]

In some embodiments, M^{a} is selected from the group consisting of:

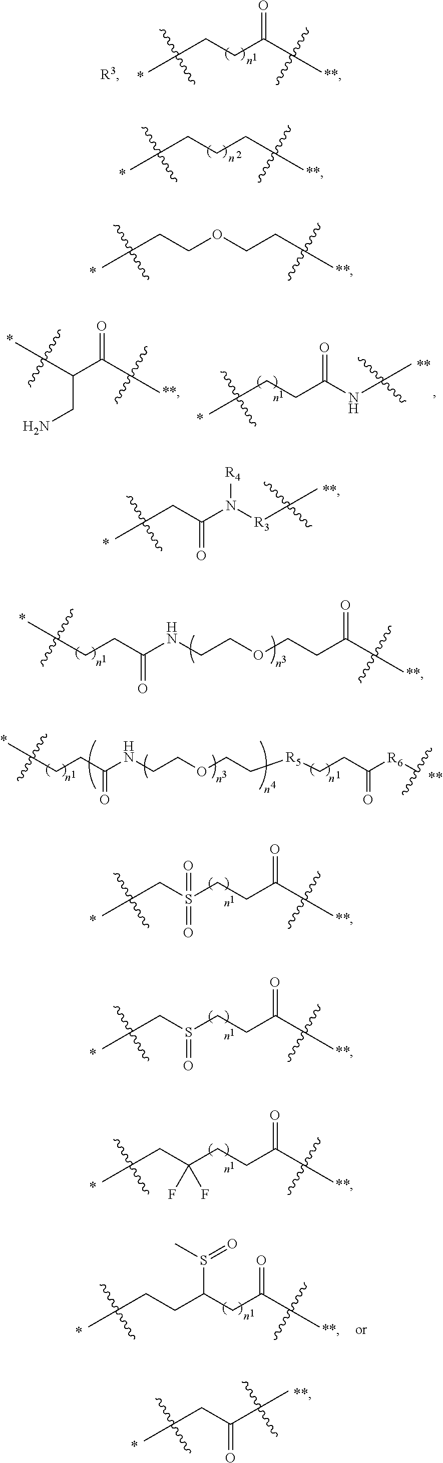

wherein
   * is the site covalently attached to L, ** is the site covalently attached to —NH-moiety;
   R³ is C₁₋₁₀ alkyl, C₁₋₁₀ heteroalkyl, C₃₋₈ cycloalkyl, —O—(C₁₋₈ alkyl)-, aryl, —C₁₋₁₀ alkyl-aryl-, -aryl-C₁₋₁₀ alkyl-, —C₁₋₁₀ alkyl-(C₃₋₈ cycloalkyl)-, —(C₃₋₈ cycloalkyl-C₁₋₁₀ alkyl)-, 4- to 14-membered heterocycloalkyl, —C₁₋₁₀ alkyl-(4- to 14-membered heterocycloalkyl)-, -(4- to 14-membered heterocycloalkyl)-C₁₋₁₀ alkyl-, —C₁₋₁₀ alkyl-C(═O)—, —C₁₋₁₀ heteroalkyl-C(═O)—, —C₃₋₈ cycloalkyl-C(═O)—, —O—(C₁₋₈ alkyl)-C(═O)—, —aryl-C(═O)—, —C₁₋₁₀ alkyl-aryl-C(═O)—, -aryl-C₁₋₁₀ alkyl-C(═O)—, —C₁₋₁₀ alkyl-(C₃₋₈ cycloalkyl)-C(═O)—, —(C₃₋₈ cycloalkyl)-C₁₋₁₀ alkyl-C(═O)—, -4- to 14-membered heterocycloalkyl-C═(O)—, —C₁₋₁₀ alkyl-(4- to 14-membered heterocycloalkyl)-C(═O)—, -(4- to 14-membered heterocycloalkyl)-C₁₋₁₀ alkyl-C(═O)—, —C₁₋₁₀ alkyl-NH—, —C₁₋₁₀ heteroalkyl-NH—, —C₃₋₈ cycloalkyl-NH—, —O—(C₁₋₈ alkyl)-NH—, -aryl-NH—, —C₁₋₁₀ alkyl-aryl-NH—, -aryl-C₁₋₁₀ alkyl-NH—, —C₁₋₁₀ alkyl-(C₃₋₈ cycloalkyl)-NH—, —(C₃₋₈ cycloalkyl)-C₁₋₁₀ alkyl-NH—, -4- to 14-membered heterocycloalkyl-NH—, —C₁₋₁₀ alkyl-(4- to 14-membered heterocycloalkyl)-NH—, -(4- to 14-membered heterocycloalkyl)-C₁₋₁₀ alkyl-NH—, —C₁₋₁₀ alkyl-S—, —C₁₋₁₀ heteroalkyl-S—, —C₃₋₈ cycloalkyl-S—, —O—C₁₋₈ alkyl-S—, -aryl-S—, —C₁₋₁₀ alkyl-aryl-S—, -aryl-C₁₋₁₀ alkyl-S—, —C₁₋₁₀ alkyl-(C₃₋₈ cycloalkyl)-S—, —(C₃₋₈ cycloalkyl)-C₁₋₁₀ alkyl-S—, -4- to 14-membered heterocycloalkyl-S—, —C₁₋₁₀ alkyl-(4- to 14-membered heterocycloalkyl)-S—, or -(4- to 14-membered heterocycloalkyl)-C₁₋₁₀ alkyl-S—;
   each R⁴ independently is hydrogen, C₁₋₆ alkyl, C₆₋₁₀ aryl, C₃₋₈ cycloalkyl, —COOH or —COO—C₁₋₆ alkyl;
   R⁵ is —C(O)—NR^{5a} or —NR^{5a}—C(O)—;
   R^{5a} is hydrogen, C₁₋₆ alkyl, C₆₋₁₀ aryl, C₃₋₈ cycloalkyl, —COOH or —COO—C₁₋₆ alkyl;
   R⁶ is a bond or —NR^{6a}—(CR^{6b}R^{6c})—C(O)—;
   R^{6a} is hydrogen, C₁₋₆ alkyl, C₆₋₁₀ aryl, C₃₋₈ cycloalkyl, —COOH or —COO—C₁₋₆ alkyl;
   each R^{6b} and R^{6c} independently is hydrogen, C₁₋₆ alkyl, C₆₋₁₀ aryl, hydroxylated C₆₋₁₀ aryl, polyhydroxylated C₆₋₁₀ aryl, 5- to 12-membered heterocycloalkyl, C₃₋₈ cycloalkyl, hydroxylated C₃₋₈ cycloalkyl, polyhydroxylated C₃₋₈ cycloalkyl or a side chain of a natural or unnatural amino acid;
   each n¹ independently is an integer from 0 to 6;
   n² is an integer from 0 to 8;
   each n³ independently is an integer from 1 to 6; and
   n⁴ is an integer from 1 to 4.

In certain embodiments, M^{a} is selected from the group consisting of:

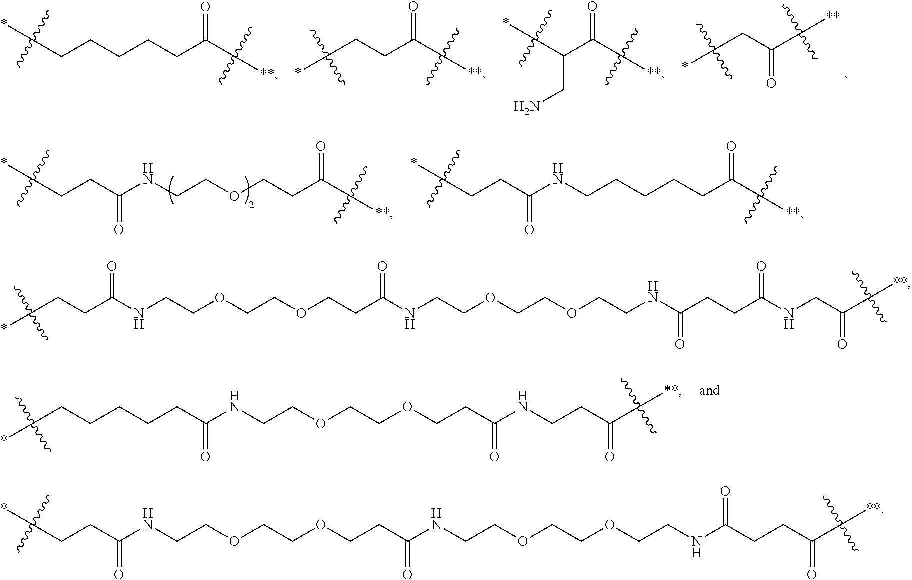

In some embodiments, G¹ and G² are independently selected from the group insisting of:

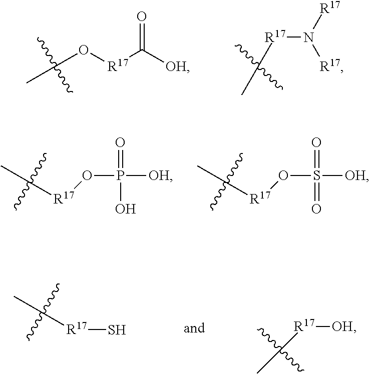

in which, each R¹⁷ is independently selected from a direct bond, alkyl, cycloalkyl, heterocycloalkyl, aryl, or heteroaryl.

In some embodiments, the polymeric scaffold provided herein has a structure of Formula (IIa) or (IIb):

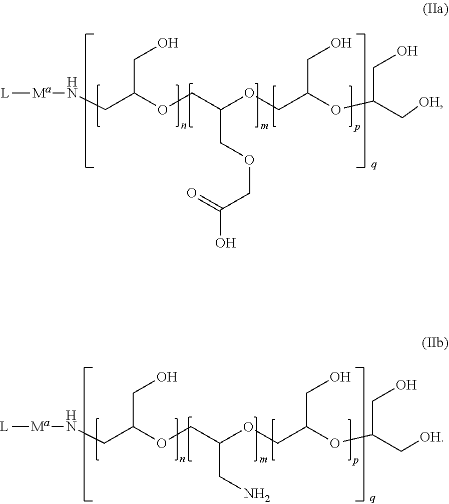

In some embodiments, the polymeric scaffold provided herein has a structure of Formula (IIc) or (IId):

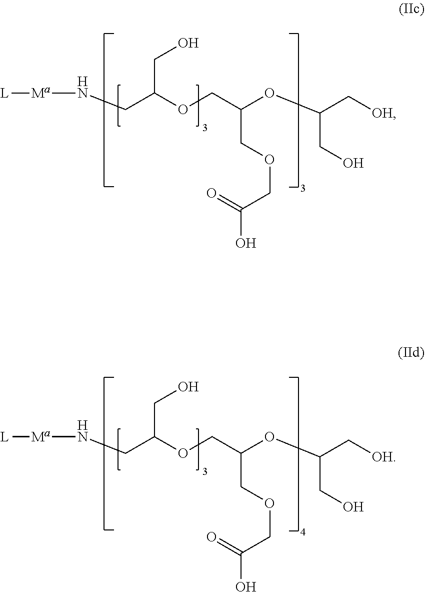

### Modified Polymer Moiety

The present disclosure also relates to modified polymer moiety that can further binds to a linker moiety and drug moiety to form the linker-polymer-drug compound provided herein.

Therefore, in another aspect, the present disclosure provides a polymeric scaffold of Formula (III):

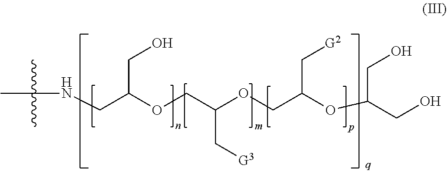

wherein,
   the polymeric scaffold comprises linear polyglycerol;
   each G² is independently a functional group capable of converting into a charged state;
   each G³ independently comprises a functional group capable of reacting with a reactive group in a drug release mechanism to connect the drug release mechanism to the linear polyglycerol;
   n is an integer from 0 to 1000;
   m is an integer from 0 to 1000;
   p is an integer from 0 to 1000; and
   q is an integer from 0 to 1000.

In some embodiments, n is 2; m is 2; and p is 2.

In some embodiments, q is an integer from 3 to 5.

In some embodiments, G¹ and G² are independently selected from the group insisting of:

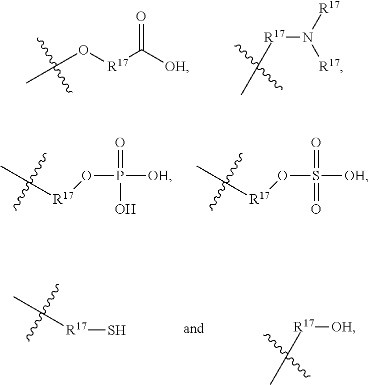

wherein each R¹⁷ is independently selected from a direct bond, alkyl, cycloalkyl, heterocycloalkyl, aryl or heteroaryl.

In some embodiments, the polymer scaffold has a structure of Formula (IIIa) or (IIIb):

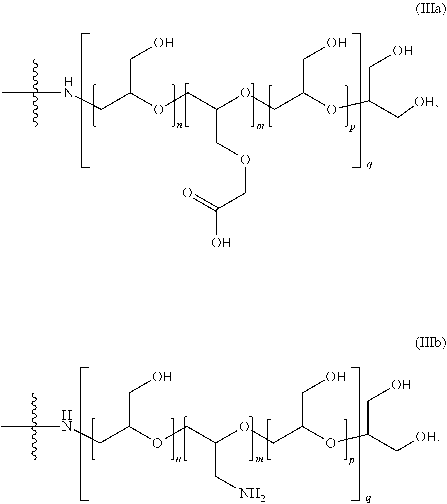

In some embodiments, a linker has a structure of Formula (IIIc) or (IIId)

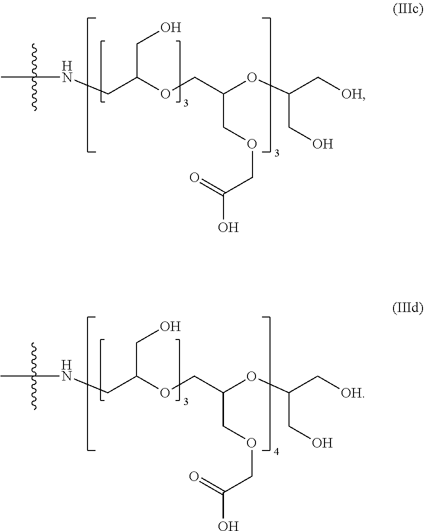

In some embodiments, the polymeric scaffold has a weight average molecular weight Mw of 1-100,000. In certain embodiments, the polymeric scaffold has a weight average molecular weight Mw of 10,000-15,000. In certain embodiments, the polymeric scaffold has a weight average molecular weight Mw of 5,000-10,000.

In some embodiments, the polymeric scaffold has a PDI of less than 1.5.

### Targeting Moiety

The targeting moiety directs the linker-polymer-drug conjugates to specific tissues, cells, or locations in a cell. The targeting moiety can direct the modified polymer in culture or in a whole organism, or both. In each case, the targeting moiety can bind to a ligand that is present on the cell surface of the targeted cell(s) with an effective specificity, affinity and avidity. In some embodiments, the targeting moiety targets the modified polymer to tissues other than the liver. In other embodiments, the targeting moiety targets the modified polymer to a specific tissue such as the liver, kidney, lung or pancreas. The targeting moiety can target the modified polymer to a target cell such as a cancer cell, such as a receptor expressed on a cell such as a cancer cell, a matrix tissue, or a protein associated with cancer such as tumor antigen. Alternatively, cells comprising the tumor vasculature may be targeted. The targeting moiety can direct the modified polymer to specific types of cells such as specific targeting to hepatocytes in the liver as opposed to Kupffer cells. In other cases, the targeting moiety can direct the modified polymer to cells of the reticular endothelial or lymphatic system, or to professional phagocytic cells such as macrophages or eosinophils.

In still other embodiments, the targeting moiety can target the modified polymer to a location within the cell, such as the nucleus, the cytoplasm, or the endosome, for example. In certain embodiments, the targeting moiety can enhance cellular binding to receptors, or cytoplasmic transport to the nucleus and nuclear entry or release from endosomes or other intracellular vesicles.

In some embodiments, the targeting moiety includes antibodies, proteins and peptides or peptide mimics.

In some embodiments, the targeting moiety comprises natural amino acids that are capable of reacting with a functional group in the linking moiety of the linker-polymer-drug conjugate to form a covalent bond. In certain embodiments, the natural amino acid includes cysteine, lysine, tyrosine, aspartic acid and glutamic acid.

In certain embodiments, the targeting moiety comprises cysteine and the targeting moiety is conjugated to the linker-polymer-drug conjugate by a covalent bond via the sulfhydryl group and a functional group of the linking moiety in the linker-polymer-drug conjugate.

In certain embodiments, the targeting moiety comprises lysine and the targeting moiety is conjugated to the linker-polymer-drug conjugate by a covalent bond via the amino group and a functional group of the linking moiety in the linker-polymer-drug conjugate.

In some embodiments, the targeting moiety may comprise non-natural amino acids that are capable of reacting with a functional group in the linking moiety of the linker-polymer-drug conjugate to form a covalent bond. In certain embodiments, the targeting moiety is conjugated to the linker-polymer-drug conjugate by a covalent bond via the amino group and a functional group of the linking moiety in the linker-polymer-drug conjugate.

In some embodiments, the targeting moiety may comprise functional groups that are capable of reacting with a functional group in the linking moiety of the linker-polymer-drug conjugate via click reaction to form a covalent bond.

In some embodiments, the targeting moiety can be antibodies or antibodies derived from Fab, Fab2, scFv or camel antibody heavy-chain fragments specific to the cell surface markers, including but not limited to, 5T4, AOC3, ALK, AXL, C242, CA-125, CCL11, CCR 5, CD2, CD3, CD4, CD5, CD15, CA15-3, CD18, CD19, CA19-9, CD20, CD22, CD23, CD25, CD28, CD30, CD31, CD33, CD37, CD38, CD40, CD41, CD44, CD44 v6, CD51, CD52, CD54, CD56, CD62E, CD62P, CD62L, CD70, CD74, CD79-B, CD80, CD125, CD138, CD141, CD147, CD152, CD 154, CD326, CEA, clumping factor, CTLA-4, CXCR2, EGFR (HER1), ErbB2, ErbB3, EpCAM, EPHA2, EPHB2, EPHB4, FGFR (i.e. FGFR1, FGFR2, FGFR3, FGFR4), FLT3, folate receptor, FAP, GD2, GD3, GPNMB, HGF, HMI.24, ICAM, ICOS-L, IGF-1 receptor, VEGFR1, EphA2, TRPV1, CFTR, gpNMB, CA9, Cripto, c-KIT, c-MET, ACE, APP, adrenergic receptor-beta2, Claudine 3, Mesothelin, MUC1, NaPi2b, NOTCH1, NOTCH2, NOTCH3, NOTCH4, RON, ROR1, PD-L1, PD-L2, B7-H3, B7-B4, IL-2 receptor, IL-4 receptor, IL-13 receptor, Trop-2, integrins (including α₄, αᵥβ₃, αᵥβ₅, αᵥβ₆, α₁β₄, α₄β₁, α₄β₇, α₅β₁, α₆β₄, α_{IIb}β₃ intergins), IFN-α, IFN-γ, IgE, IgE, IGF-1 receptor, IL-1, IL-12, IL-23, IL-13, IL-22, IL-4, IL-5, IL-6, interferon receptor, ITGB2 (CD18), LFA-1 (CD11a), L-selectin (CD62L), mucin, MUC1, myostatin, NCA-90, NGF, PDGFRα, phosphatidylserine, prostatic carcinoma cell, Pseudomonas aeruginosa, rabies, RANKL, respiratory syncytial virus, Rhesus factor, SLAMF7, sphingosine-1-phosphate, TAG-72, T-cell receptor, tenascin C, TGF-1, TGF-β2, TGF-β, TNF-α, TRAIL-R1, TRAIL-R2, tumor antigen CTAA16.88, VEGF-A, VEGFR2, vimentin, and the like.

In certain embodiments, the antibodies or antibody derived from Fab, Fab2, scFv or camel antibody heavy-chain fragments specific to the cell surface markers include CA-125, C242, CD3, CD19, CD22, CD25, CD30, CD31, CD33, CD37, CD40, CD44, CD51, CD54, CD56, CD62E, CD62P, CD62L, CD70, CD138, CD141, CD326, CEA, CTLA-4, EGFR (HER1), ErbB2, ErbB3, FAP, folate receptor, IGF-1 receptor, GD3, GPNMB, HGF, VEGF-A, VEGFR2, VEGFR1, EphA2, EpCAM, 5T4, TAG-72, tenascin C, TRPV1, CFTR, gpNMB, CA9, Cripto, ACE, APP, PDGFR α, phosphatidylserine, prostatic carcinoma cells, adrenergic receptor-beta2, Claudine 3, mucin, MUC1, Mesothelin, IL-2 receptor, IL-4 receptor, IL-13 receptor and integrins (including αᵥβ₃, βᵥβ₅, αᵥβ₆, α₁β₄, α₄β₁, α₅β₁, α₆β₄ intergins), tenascin C, TRAIL-R2 and vimentin.

Exemplary antibodies include 3F8, abagovomab, abciximab (REOPRO), adalimumab (HUMIRA), adecatumumab, afelimomab, afutuzumab, alacizumab, ALD518, alemtuzumab (CAMPATH), altumomab, amatuximab, anatumomab, anrukinzumab, apolizumab, arcitumomab (CEA-SCAN), aselizumab, atlizumab (tocilizumab, Actemra, RoActemra), atorolimumab, bapineuzumab, basiliximab (Simulect), bavituximab, bectumomab (LYMPHOSCAN), belimumab (BENLYSTA), benralizumab, bertilimumab, besilesomab (SCINITIMUN), bevacizumab (AVASTIN), biciromab (FIBRISCINT), bivatuzumab, blinatumomab, brentuximab, briakinumab, canakinumab (ILARIS), cantuzumab, capromab, catumaxomab (REMOVAB), CC49, cedelizumab, certolizumab, cetuximab (ERBITUX), citatuzumab, cixutumumab, clenoliximab, clivatuzumab, conatumumab, CR6261, dacetuzumab, daclizumab (ZENAPAX), daratumumab, denosumab (PROLIA), detumomab, dorlimomab, dorlixizumab, ecromeximab, eculizumab (SOLIRIS), edobacomab, edrecolomab (PANOREX), efalizumab (RAPTIVA), efungumab (MYCOGRAB), elotuzumab, elsilimomab, enlimomab, epitumomab, epratuzumab, erlizumab, ertumaxomab (REXOMUN), etaracizumab (ABEGRIN), exbivirumab, fanolesomab (NEUTROSPEC), faralimomab, farletuzumab, felvizumab, fezakinumab, figitumumab, fontolizumab (HuZAF), foravirumab, fresolimumab, galiximab, gantenerumab, gavilimomab, gemtuzumab, girentuximab, glembatumumab, golimumab (SIMPONI), gomiliximab, ibalizumab, ibritumomab, igovomab (INDIMACIS-125), imciromab (MYOSCINT), infliximab (REMICADE), intetumumab, inolimomab, inotuzumab, ipilimumab, iratumumab, keliximab, labetuzumab (CEA-CIDE), lebrikizumab, lemalesomab, lerdelimumab, lexatumumab, libivirumab, lintuzumab, lucatumumab, lumiliximab, mapatumumab, maslimomab, matuzumab, mepolizumab (BOSATRIA), metelimumab, milatuzumab, minretumomab, mitumomab, morolimumab, motavizumab (NUMAX), muromonab-CD3 (ORTHOCLONE OKT3), nacolomab, naptumomab, natalizumab (TYSABRI), nebacumab, necitumumab, nerelimomab, nimotuzumab (THERACIM), nofetumomab, ocrelizumab, odulimomab, ofatumumab (ARZERRA), olaratumab, omalizumab (XOLAIR), ontecizumab, oportuzumab, oregovomab (OVAREX), otelixizumab, pagibaximab, palivizumab (SYNAGIS), panitumumab (VECTIBIX), panobacumab, pascolizumab, pemtumomab (THERAGYN), pertuzumab (OMNITARG), pexelizumab, pintumomab, priliximab, pritumumab, PRO 140, rafivirumab, ramucirumab, ranibizumab (LUCENTIS), raxibacumab, regavirumab, reslizumab, rilotumumab, rituximab (RITUXAN), robatumumab, rontalizumab, rovelizumab (LEUKARREST), ruplizumab (ANTOVA), sacituzumab, satumomab pendetide, sevirumab, sibrotuzumab, sifalimumab, siltuximab, siplizumab, solanezumab, sonepcizumab, sontuzumab, stamulumab, sulesomab (LEUKOSCAN), tacatuzumab (AFP-CIDE), tetraxetan, tadocizumab, talizumab, tanezumab, taplitumomab paptox, tefibazumab (AUREXIS), telimomab, tenatumomab, teneliximab, teplizumab, TGN1412, ticilimumab (tremelimumab), tigatuzumab, TNX-650, tocilizumab (atlizumab, ACTEMRA), toralizumab, tositumomab (BEXXAR), trastuzumab (HERCEPTIN), tremelimumab, tucotuzumab, tuvirumab, urtoxazumab, ustekinumab (STELERA), vapaliximab, vedolizumab, veltuzumab, vepalimomab, visilizumab (NUVION), volociximab (HUMASPECT), votumumab, zalutumumab (HuMEX-EGFr), zanolimumab (HuMAX-CD4), ziralimumab and zolimomab.

In some embodiments, the antibodies are directed to cell surface markers for 5T4, CA-125, CEA, CD3, CD19, CD20, CD22, CD30, CD33, CD40, CD44, CD51, CTLA-4, EpCAM, HER2, EGFR (HER1), FAP, folate receptor, HGF, integrin αvβ3, integrin α5β1, IGF-1 receptor, GD3, GPNMB, mucin, MUC1, phosphatidylserine, prostatic carcinoma cells, PDGFR α, TAG-72, tenascin C, TRAIL-R2, VEGF-A and VEGFR2. In this embodiment the antibodies are abagovomab, adecatumumab, alacizumab, altumomab, anatumomab, arcitumomab, bavituximab, bevacizumab (AVASTIN), bivatuzumab, blinatumomab, brentuximab, cantuzumab, catumaxomab, capromab, cetuximab, citatuzumab, clivatuzumab, conatumumab, dacetuzumab, edrecolomab, epratuzumab, ertumaxomab, etaracizumab, farletuzumab, figitumumab, gemtuzumab, glembatumumab, ibritumomab, igovomab, intetumumab, inotuzumab, labetuzumab, lexatumumab, lintuzumab, lucatumumab, matuzumab, mitumomab, naptumomab estafenatox, necitumumab, oportuzumab, oregovomab, panitumumab, pemtumomab, pertuzumab, pritumumab, rituximab (RITUXAN), rilotumumab, robatumumab, satumomab, sibrotuzumab, taplitumomab, tenatumomab, tenatumomab, ticilimumab (tremelimumab), tigatuzumab, trastuzumab (HERCEPTIN), tositumomab, tremelimumab, tucotuzumab celmoleukin, volociximab and zalutumumab.

In certain embodiments, the antibodies directed to cell surface markers for HER2 are pertuzumab or trastuzumab and for EGFR (HER1) the antibody is cetuximab or panitumumab; and for CD20 the antibody is rituximab and for VEGF-A is bevacizumab and for CD-22 the antibody is epratuzumab or veltuzumab and for CEA the antibody is labetuzumab.

Exemplary peptides or peptide mimics include integrin targeting peptides (RGD peptides), LHRH receptor targeting peptides, ErbB2 (HER2) receptor targeting peptides, prostate specific membrane bound antigen (PSMA) targeting peptides, lipoprotein receptor LRP1 targeting, ApoE protein derived peptides, ApoA protein peptides, somatostatin receptor targeting peptides, chlorotoxin derived peptides, and bombesin.

In specific embodiments the peptides or peptide mimics are LHRH receptor targeting peptides and ErbB2 (HER2) receptor targeting peptides.

Exemplary proteins comprise insulin, transferrin, fibrinogen-gamma fragment, thrombospondin, claudin, apolipoprotein E, Affibody molecules such as, for example, ABY-025, Ankyrin repeat proteins, ankyrin-like repeats proteins and synthetic peptides.

In some embodiments, the targeting moiety-linker-polymer-drug conjugates comprise broad spectrum cytotoxins in combination with cell surface markers for HER2 such as pertuzumab or trastuzumab; for EGFR such as cetuximab and panitumumab; for CEA such as labetuzumab; for CD20 such as rituximab; for VEGF-A such as bevacizumab; or for CD-22 such as epratuzumab or veltuzumab.

In other embodiments, the targeting moiety-linker-drug-polymer conjugates comprise combinations of two or more targeting moieties, such as, for example, combination of bispecific antibodies directed to the EGF receptor (EGFR) on tumor cells and to CD3 and CD28 on T cells; combination of antibodies or antibody derived from Fab, Fab2, scFv or camel antibody heavy-chain fragments and peptides or peptide mimetics; combination of antibodies or antibody derived from Fab, Fab2, scFv or camel antibody heavy-chain fragments and proteins; combination of two bispecific antibodies such as CD3×CD19 plus CD28×CD22 bispecific antibodies.

In other embodiments, the targeting moiety-linker-drug-polymer conjugates comprise targeting moieties which are antibodies against antigens, such as, for example, Trastuzumab, Cetuximab, Rituximab, Bevacizumab, Epratuzumab, Veltuzumab, Labetuzumab, B7-H4, B7-H3, CA125, CD33, CXCR2, EGFR, FGFR1, FGFR2, FGFR3, FGFR4, HER2, NaPi2b, c-Met, NOTCH1, NOTCH2, NOTCH3, NOTCH4, PD-L1, c-Kit, MUC1 and 5T4.

In certain embodiments, the targeting moiety-linker-drug-polymer conjugates comprise targeting moieties which are antibodies against 5T4, such as, for example a humanized anti-5T4 scFvFc antibody.

Examples of suitable 5T4 targeting ligands or immunoglobulins include those which are commercially available, or have been described in the patent or non-patent literature, e.g., U.S. Pat. Nos. 8,044,178, 8,309,094, 7,514,546, EP1036091 (commercially available as TroVax™, Oxford Biomedica), EP2368914A1, WO 2013041687 A1 (Amgen), US 2010/0173382, and P. Sapra, et al., Mol. Cancer Ther. 2013, 12:38-47. An anti-5T4 antibody is disclosed in U.S. Provisional Application No. 61/877,439, filed Sep. 13, 2013 and U.S. Provisional Application No. 61/835,858, filed Jun. 17, 2013. The contents of each of the patent documents and scientific publications are herein incorporated by reference in their entireties.

As used herein, the term “5T4 antigen-binding portion” refers to a polypeptide sequence capable of selectively binding to a 5T4 antigen. In exemplary conjugates, the 5T4 antigen-binding portion generally comprises a single chain scFv-Fc form engineered from an anti-5T4 antibody. A single-chain variable fragment (scFv-Fc) is a fusion protein of the variable regions of the heavy (VH) and light chains (VL) of an immunoglobulin, connected with a linker peptide, and further connected to an Fc region comprising a hinge region and CH2 and CH3 regions of an antibody (any such combinations of antibody portions with each other or with other peptide sequences is sometimes referred to herein as an “immunofusion” molecule). Within such a scFvFc molecule, the scFv section may be C-terminally linked to the N-terminus of the Fc section by a linker peptide.

In some embodiments, the Fv portion of the 5T4 antigen-binding portion may be engineered by well-known molecular biology techniques to comprise one or more amino acid substitutions in the VH region. The Fc portion of the 5T4 antigen binding portion preferably comprises a polypeptide sequence engineered from the human hinge, CH2 and CH3 regions of an anti-5T4 antibody.

In some embodiments, the targeting moiety-linker-drug-polymer conjugates comprise targeting moieties which are antibodies against TROP2. TROP2 is a member of the TACSTD family expressed in human trophoblasts and is a single-pass transmembrane type 1 cell membrane protein involved in immune resistance, which is common to human trophoblasts and cancer cells.

The TROP2 antibody useful in the targeting moiety-linker-drug-polymer conjugates provided herein can be obtained using a method usually carried out in the art, which involves immunizing an animal with TROP2 or an arbitrary polypeptide selected from the amino acid sequence of TROP2, and collecting and purifying the antibody produced in vivo. The biological species of TROP2 to be used as an antigen is not limited to being human, and an animal can be immunized with TROP2 derived from an animal other than humans such as a mouse or a rat. In this case, by examining the cross-reactivity between an antibody binding to the obtained heterologous TROP2 and human TROP2, an antibody applicable to a human disease can be selected. Further, a monoclonal antibody can be obtained from a hybridoma established by fusing antibody-producing cells which produce an antibody against TROP2 with myeloma cells according to a known method (for example, Kohler and Milstein, Nature, (1975) 256, pp. 495-497; Rennet, R. ed., Monoclonal Antibodies, pp. 365-367, Plenum Press, N.Y. (1980)).

In some embodiment, the Trop2 antibodies useful in the targeting moiety-linker-drug-polymer conjugates provided herein are commercially available from a number of sources, and include but are not limited to LS-C126418, LS-C178765, LS-C126416, LS-C126417 (LifeSpan Biosciences, Inc., Seattle, WA); 10428-MM01, 10428-MM02, 10428—R001, 10428—R030 (Sino Biological Inc., Beijing, China); MR54 (eBioscience, San Diego, CA); sc-376181, sc-376746, Santa Cruz Biotechnology (Santa Cruz, CA); MM0588-49D6, (Novus Biologicals, Littleton, CO); ab79976, and ab89928 (ABCAM®, Cambridge, MA).

Other Trop2 antibodies useful in the targeting moiety-linker-drug-polymer conjugates provided herein have been disclosed in the patent literature. For example, U.S. Publ. No. 2013/0089872 discloses Trop2 antibodies K5-70 (Accession No. FERM BP-11251), K5-107 (Accession No. FERM BP—11252), K5-116-2-1 (Accession No. FERM BP-11253), T6-16 (Accession No. FERM BP-11346), and T5-86 (Accession No. FERM BP—11254), deposited with the International Patent Organism Depositary, Tsukuba, Japan. U.S. Pat. No. 5,840,854 disclosed the Trop2 monoclonal antibody BR 110 (ATCC No. HB11698). U.S. Pat. No. 7,420,040 disclosed a Trop2 antibody produced by hybridoma cell line AR47A6.4.2, deposited with the ID AC (International Depository Authority of Canada, Winnipeg, Canada) as accession number 141205-05. U.S. Pat. No. 7,420,041 disclosed a Trop2 antibody produced by hybridoma cell line AR52A301.5, deposited with the IDAC as accession number 141205-03. U.S. Publ. No. 2013/0122020 disclosed Trop2 antibodies 3E9, 6G11, 7E6, 15E2, 18B1. Hybridomas encoding a representative antibody were deposited with the American Type Culture Collection (ATCC), Accession Nos. PTA-12871 and PTA-12872. U.S. Pat. No. 8,715,662 discloses Trop2 antibodies produced by hybridomas deposited at the AID-ICLC (Genoa, Italy) with deposit numbers PD 08019, PD 08020 and PD 08021. U.S. Patent Application Publ. No. 20120237518 discloses Trop2 antibodies 77220, KM4097 and KM4590. U.S. Pat. No. 8,309,094 (Wyeth) discloses antibodies A1 and A3, identified by sequence listing. U.S. Pat. No. 10,227,417 discloses a number of Trop2 antibodies identified by sequence listing. The Examples section of each patent or patent application cited above in this paragraph is incorporated herein by reference. For non-patent publications, Lipinski et al. (1981, Proc Natl. Acad Sci USA, 78:5147-50) disclosed Trop-2 antibodies 162-25.3 and 162-46.2. The PriE11 Trop2 antibody was reported to recognize a unique epitope on Trop2 (Ikeda et al., Biochem Biophys Res Comm 458:877-82).

In some embodiments, the targeting moiety-linker-drug-polymer conjugates comprise targeting moieties which are antibodies against HER2. HER2 is one of the oncogene products of a typical growth factor receptor oncogene identified as human epidermal cell growth factor receptor 2-related oncogene, and is a transmembrane receptor protein having a molecular weight of 185 kDa and having a tyrosine kinase domain. HER2 is a member of the EGFR family consisting of HER1 (EGFR, ErbB-1), HER2 (neu, ErbB-2), HER3 (ErbB-3), and HER4 (ErbB-4) and is known to be autophosphorylated at intracellular tyrosine residues by its homodimer formation or heterodimer formation with another EGFR receptor HER1, HER3, or HER4 and is itself activated in that manner, thereby playing an important role in cell growth, differentiation, and survival in normal cells and tumor cells.

The HER2 antibody useful in the targeting moiety-linker-drug-polymer conjugates provided herein are not particularly limited. The HER2 antibody can be obtained according to, for example, a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. Alternatively, antibody-producing cells which produce antibodies against the antigen are fused with myeloma cells according to a method known in the art (e.g., Kohler and Milstein, Nature (1975) 256, p. 495-497; and Kennet, R. ed., Monoclonal Antibodies, p. 365-367, Plenum Press, N.Y. (1980)) to establish hybridomas, from which monoclonal antibodies can in turn be obtained.

Examples of the HER2 antibody as used herein can include, but not limited to, pertuzumab (International Patent Publication No. WO 01/00245), trastuzumab (U.S. Pat. No. 5,821,337), the antibodies identified by sequence listing in U.S. Publ. No. 2019/0077880. However, the HER2 antibody useful herein is not limited thereto as long as it is a HER2 antibody specifically binding to HER2, and more preferably having an activity of internalizing in HER2-expressing cells by binding to HER2.

### Conjugate

In one aspect, there is provided a polymeric scaffold of Formula (IV):

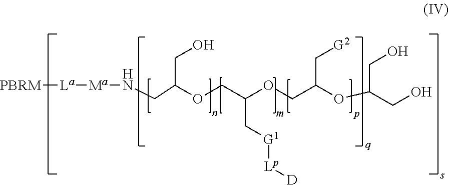

wherein
   the polymeric scaffold comprises linear polyglycerol;
   each L^{a} is independently a divalent moiety connecting the targeting moiety to M^{a};
   each M^{a} is independently a stretcher connecting L^{a} to —NH— moiety;
   each G¹ is independently a functional group connecting L^{p} to the linear polyglycerol;
   each L^{p} is independently a drug release mechanism between a therapeutic agent moiety D and G¹;
   each D is independently a therapeutic agent moiety;
   each G² is independently a functional group capable of converting into a charged state;
   n is an integer from 0 to 1000;
   m is an integer from 0 to 1000;
   p is an integer from 0 to 1000;
   q is an integer from 0 to 1000; and
   s is an integer from 1 to 8.

In some embodiments, Wₚ is capable of reacting with a functional group on the targeting moiety with a click reaction.

In certain embodiments, Wₚ is selected from the group consisting of:

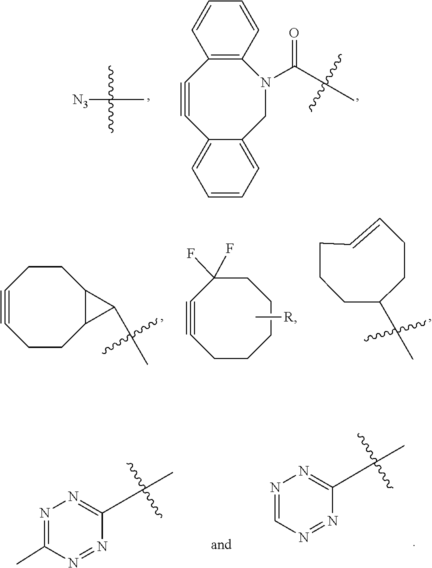

In some embodiments, Wₚ is capable of reacting with amino acids on the targeting moiety.

In certain embodiments, Wₚ is capable of reacting with amino acids on the targeting moiety, and the amino acids are natural amino acids, non-natural amino acids or combination thereof. In certain embodiments, the natural amino acid may comprise cysteine, lysine, tyrosine, aspartic acid and glutamic acid.

In some embodiments, Wₚ is capable of reacting with one or more cysteines on the targeting moiety.

In certain embodiments, Wₚ is capable of reacting with one or more cysteines on the targeting moiety and each Wₚ is selected from the group consisting of:

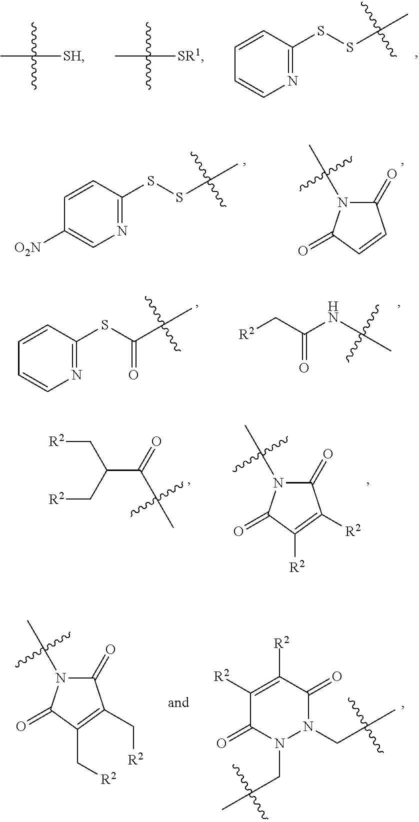

wherein R¹ is a sulfur protecting group, and each R² is independently a leaving group.

In certain embodiments, each R² is independently selected from halo or R^{2a}C(O)O—, in which R^{2a} is hydrogen, aliphatic, heteroaliphatic, cycloalkyl, heterocycloalkyl, aryl or heteroaryl.

In some embodiments, Wₚ is capable of reacting with one or more lysines on the targeting moiety.

In certain embodiments, Wₚ is capable of reacting with one or more lysines on the targeting moiety, and each Wₚ is independently selected from the group consisting of:

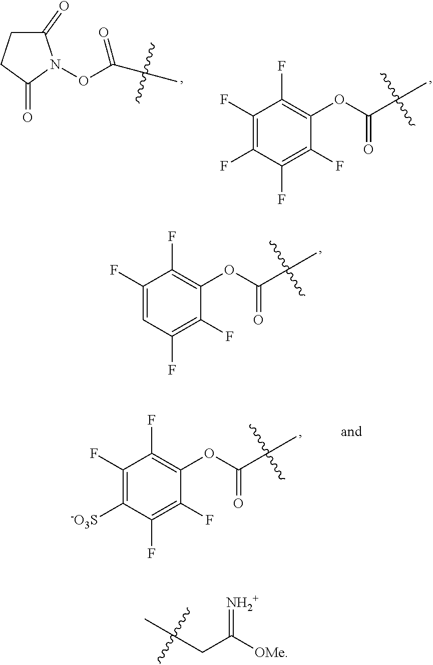

In some embodiments, Wₚ is capable of reacting with one or more non-natural amino acids on the targeting moiety.

In certain embodiments, Wₚ is capable of reacting with one or more non-natural amino acids on the targeting moiety and each Wₚ is independently selected from:

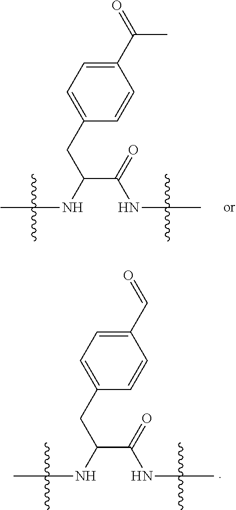 [SMILES: *NC(=O)C(Cc1ccc(C(C)=O)cc1)N* ; *NC(=O)C(Cc1ccc(C=O)cc1)N*]

In some embodiments, M^{a} is selected from the group consisting of:

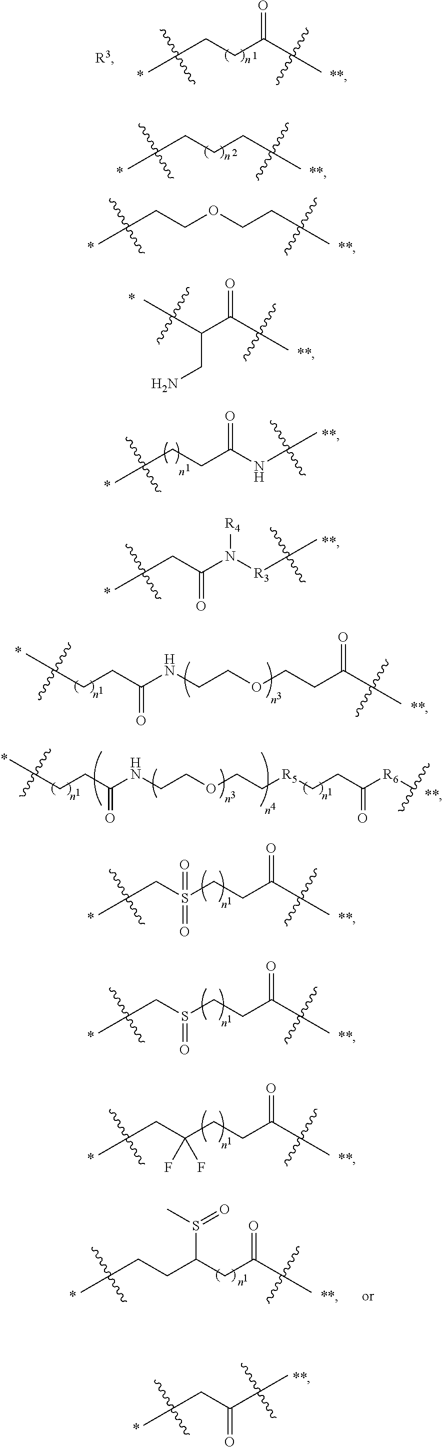

wherein
   * is the site covalently attached to L, ** is the site covalently attached to —NH-moiety;
   R³ is C₁₋₁₀ alkyl, C₁₋₁₀ heteroalkyl, C₃₋₈ cycloalkyl, —O—(C₁₋₈ alkyl)-, aryl, —C₁₋₁₀ alkyl-aryl-, -aryl-C₁₋₁₀ alkyl-, —C₁₋₁₀ alkyl-(C₃₋₈ cycloalkyl)-, —(C₃₋₈ cycloalkyl-C₁₋₁₀ alkyl)-, 4- to 14-membered heterocycloalkyl, —C₁₋₁₀ alkyl-(4- to 14-membered heterocycloalkyl)-, -(4- to 14-membered heterocycloalkyl)-C₁₋₁₀ alkyl-, —C₁₋₁₀ alkyl-C(═O)—, —C₁₋₁₀ heteroalkyl-C(═O)—, —C₃₋₈ cycloalkyl-C(═O)—, —O—(C₁₋₈ alkyl)-C(═O)—, -aryl-C(═O)—, —C₁₋₁₀ alkyl-aryl-C(═O)—, -aryl-C₁₋₁₀ alkyl-C(═O)—, —C₁₋₁₀ alkyl-(C₃₋₈ cycloalkyl)-C(═O)—, —(C₃₋₈ cycloalkyl)-C₁₋₁₀ alkyl-C(═O)—, -4- to 14-membered heterocycloalkyl-C═(O)—, —C₁₋₁₀ alkyl-(4- to 14-membered heterocycloalkyl)-C(═O)—, -(4- to 14-membered heterocycloalkyl)-C₁₋₁₀ alkyl-C(═O)—, —C₁₋₁₀ alkyl-NH—, —C₁₋₁₀ heteroalkyl-NH—, —C₃₋₈ cycloalkyl-NH—, —O—(C₁₋₈ alkyl)-NH—, -aryl-NH—, —C₁₋₁₀ alkyl-aryl-NH—, -aryl-C₁₋₁₀ alkyl-NH—, —C₁₋₁₀ alkyl-(C₃₋₈ cycloalkyl)-NH—, —(C₃₋₈ cycloalkyl)-C₁₋₁₀ alkyl-NH—, -4- to 14-membered heterocycloalkyl-NH—, —C₁₋₁₀ alkyl-(4- to 14-membered heterocycloalkyl)-NH—, -(4- to 14-membered heterocycloalkyl)-C₁₋₁₀ alkyl-NH—, —C₁₋₁₀ alkyl-S—, —C₁₋₁₀ heteroalkyl-S—, —C₃₋₈ cycloalkyl-S—, —O—C₁₋₈ alkyl-S—, -aryl-S—, —C₁₋₁₀ alkyl-aryl-S—, -aryl-C₁₋₁₀ alkyl-S—, —C₁₋₁₀ alkyl-(C₃₋₈ cycloalkyl)-S—, —(C₃₋₈ cycloalkyl)-C₁₋₁₀ alkyl-S—, -4- to 14-membered heterocycloalkyl-S—, —C₁₋₁₀ alkyl-(4- to 14-membered heterocycloalkyl)-S—, or -(4- to 14-membered heterocycloalkyl)-C₁₋₁₀ alkyl-S—;
   each R⁴ independently is hydrogen, C₁₋₆ alkyl, C₆₋₁₀ aryl, C₃₋₈ cycloalkyl, —COOH or —COO—C₁₋₆ alkyl;
   R⁵ is —C(O)—NR^{5a} or —NR^{5a}—C(O)—;
   R^{5a} is hydrogen, C₁₋₆ alkyl, C₆₋₁₀ aryl, C₃₋₈ cycloalkyl, —COOH or —COO—C₁₋₆ alkyl;
   R⁶ is a bond or —NR^{5a}—(CR^{6b}R^{6c})—C(O)—;
   R^{6a} is hydrogen, C₁₋₆ alkyl, C₆₋₁₀ aryl, C₃₋₈ cycloalkyl, —COOH or —COO—C₁₋₆ alkyl;
   each R^{6b} and R^{6c} independently is hydrogen, C₁₋₆ alkyl, C₆₋₁₀ aryl, hydroxylated C₆₋₁₀ aryl, polyhydroxylated C₆₋₁₀ aryl, 5- to 12-membered heterocycloalkyl, C₃₋₈ cycloalkyl, hydroxylated C₃₋₈ cycloalkyl, polyhydroxylated C₃₋₈ cycloalkyl or a side chain of a natural or unnatural amino acid;
   each n¹ independently is an integer from 0 to 6;
   n² is an integer from 0 to 8;
   each n³ independently is an integer from 1 to 6; and
   n⁴ is an integer from 1 to 4.

In certain embodiments, M^{a} is selected from the group consisting of:

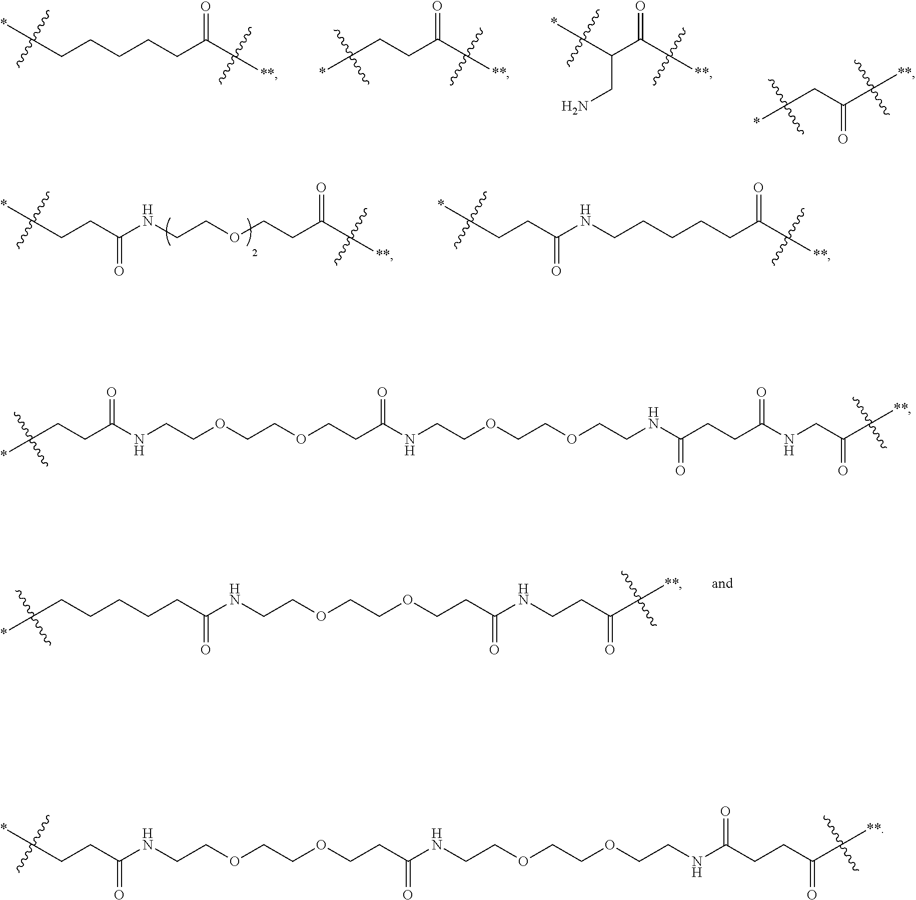

In some embodiments, G¹ is selected from the group consisting of:

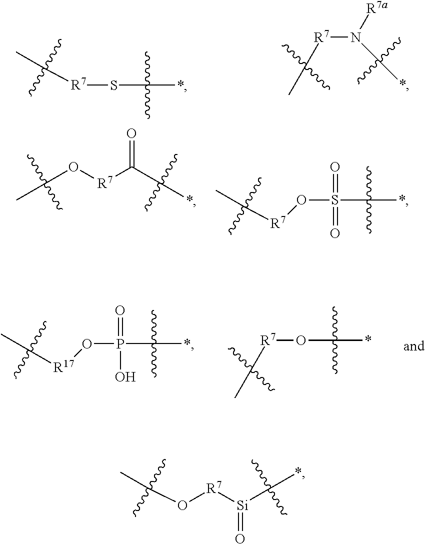

wherein * is the site covalently attached to L^{p}, each R⁷ is independently selected from a direct bond, alkyl, cycloalkyl, heterocycloalkyl, aryl, or heteroaryl, and R^{7a} is selected from hydrogen, an aliphatic, heteroaliphatic, cycloalkyl, or heterocycloalkyl moiety.

In some embodiments, G¹ is selected from the group consisting of:

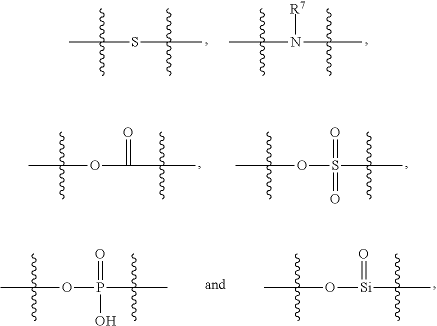

wherein R⁷ is selected from hydrogen, aliphatic, heteroaliphatic, cycloalkyl, heterocycloalkyl, aryl or heteroaryl.

In some embodiments, each L^{p} independently comprises a labile structure.

In certain embodiments, the labile structure is selected from hydrolytically labile structures or enzymatic labile structures.

In certain embodiments, the hydrolytically labile structure is selected from the group consisting of:

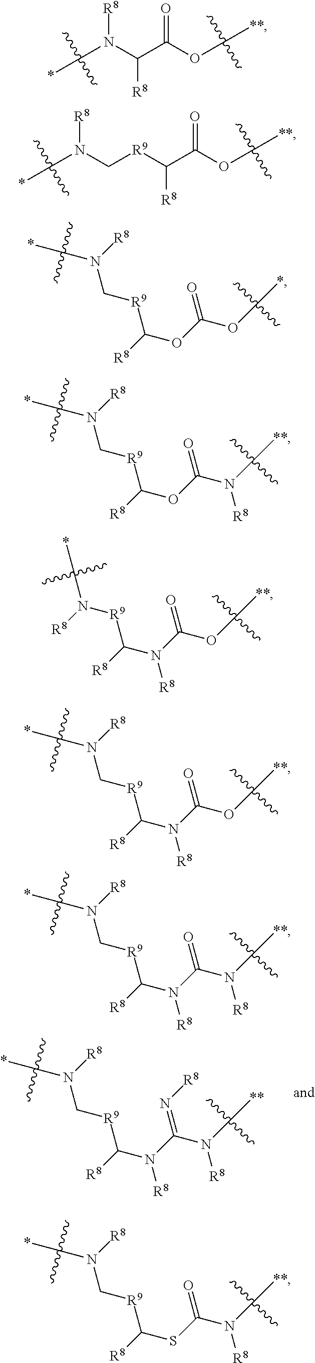

wherein * is the site covalently attached to G¹, ** is the site covalently attached to D, R⁸ is selected from hydrogen, alky or aryl, R⁹ is selected from aliphatic, heteroaliphatic, cycloalkyl, heterocycloalkyl, aryl, or heteroaryl.

In certain embodiments, the hydrolytically labile structure is selected from the group consisting of:

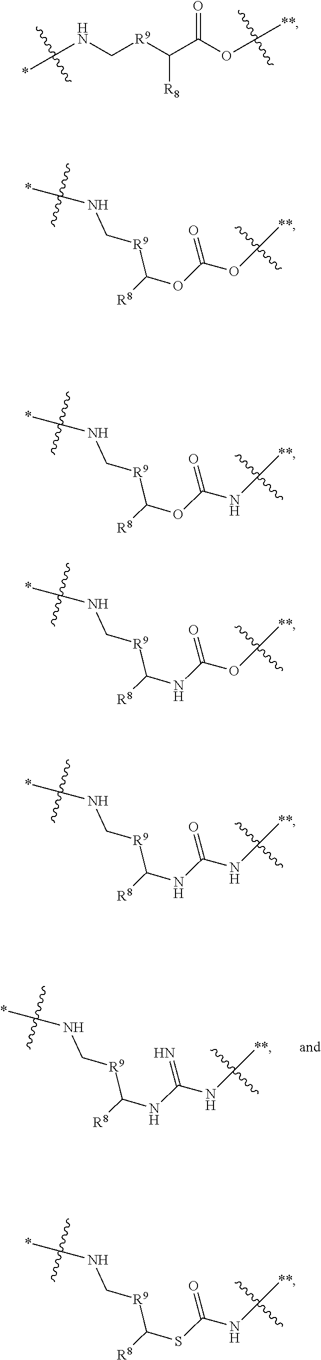

wherein * is the site covalently attached to G¹, ** is the site covalently attached to D, R⁸ is selected from hydrogen, alky or aryl, R⁹ is selected from aliphatic, heteroaliphatic, cycloalkyl, heterocycloalkyl, aryl, or heteroaryl.

In some embodiments, G¹ is

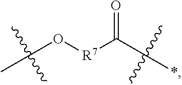

wherein * is the site covalently attached to L^{p}, R⁷ is alkyl.

In certain embodiments, -G¹-L^{p}-D is

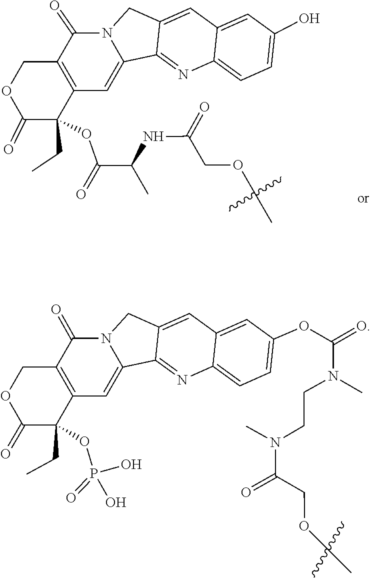 [SMILES: *OCC(=O)N[C@@H](C)C(=O)O[C@]1(CC)C(=O)OCc2c1cc1n(c2=O)Cc2cc3cc(O)ccc3nc2-1 ; *OCC(=O)N(C)CCN(C)C(=O)Oc1ccc2nc3c(cc2c1)Cn1c-3cc2c(c1=O)COC(=O)[C@@]2(CC)OP(=O)(O)O]

In some embodiments, the enzymatic labile structure is liable to enzymes selected from Cathepsin B, phosphatase, sulfatase, or glucuronidase.

In certain embodiments, the enzymatic labile structure is liable to cathepsin B and is selected from —Z— or

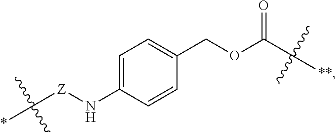

wherein Z is a substrate for cathepsin B comprising 2 to 4 amino acids.

In some embodiments, G¹ is

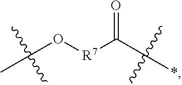

wherein * is the site covalently attached to L^{p}, R⁷ is alkyl.

In certain embodiments, -G¹-L^{p}-D is

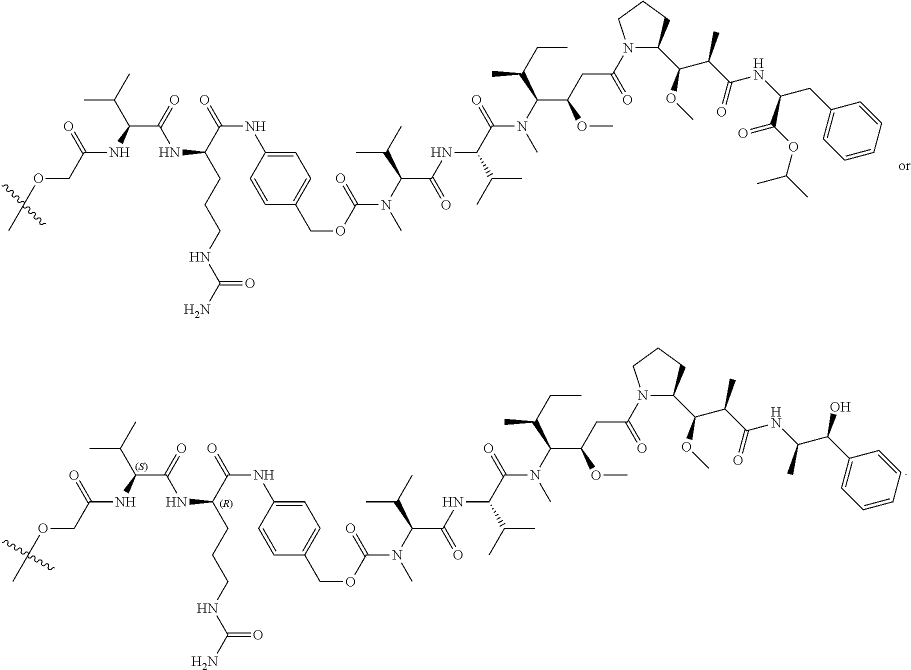

In certain embodiments, the enzymatic labile structure is liable to glucuronidase and is

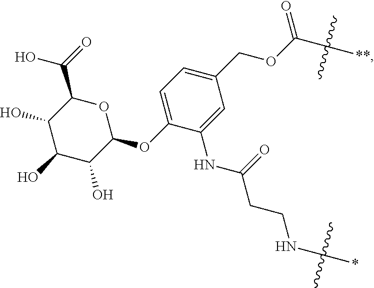

wherein * is the site covalently attached to G¹, ** is the site covalently attached to D.

In some embodiments, G¹ is

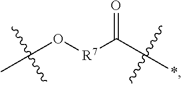

wherein * is the site covalently attached to L^{p}, R⁷ is alkyl.

In certain embodiments, G¹ is

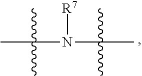

wherein R⁷ is selected from hydrogen, aliphatic, heteroaliphatic, cycloalkyl, or heterocycloalkyl.

In certain embodiments, -L^{p}-D is selected from:

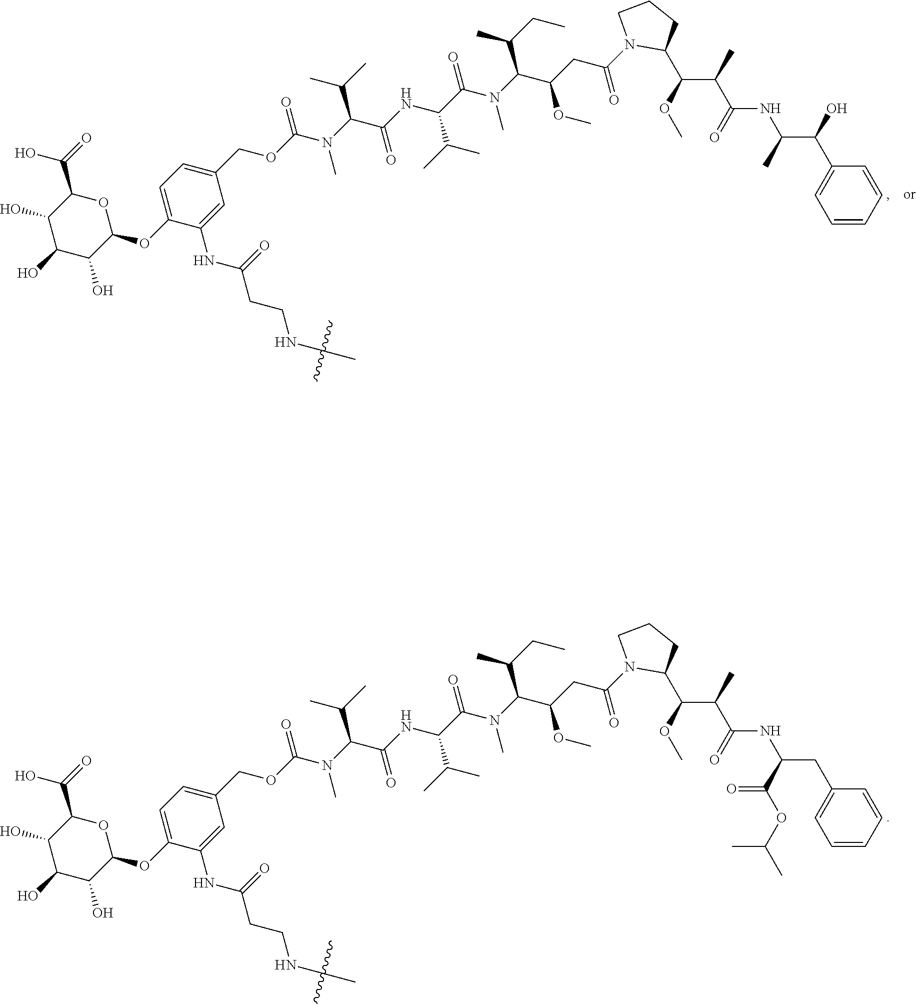 [SMILES: *NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)ccc1O[C@@H]1O[C@H](C(=O)O)[C@@H](O)[C@H](O)[C@H]1O ; *NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)C(=O)OC(C)C)OC)C(C)C)C(C)C)ccc1O[C@@H]1O[C@H](C(=O)O)[C@@H](O)[C@H](O)[C@H]1O]

In certain embodiments, -G¹-L^{p}-D is selected from:

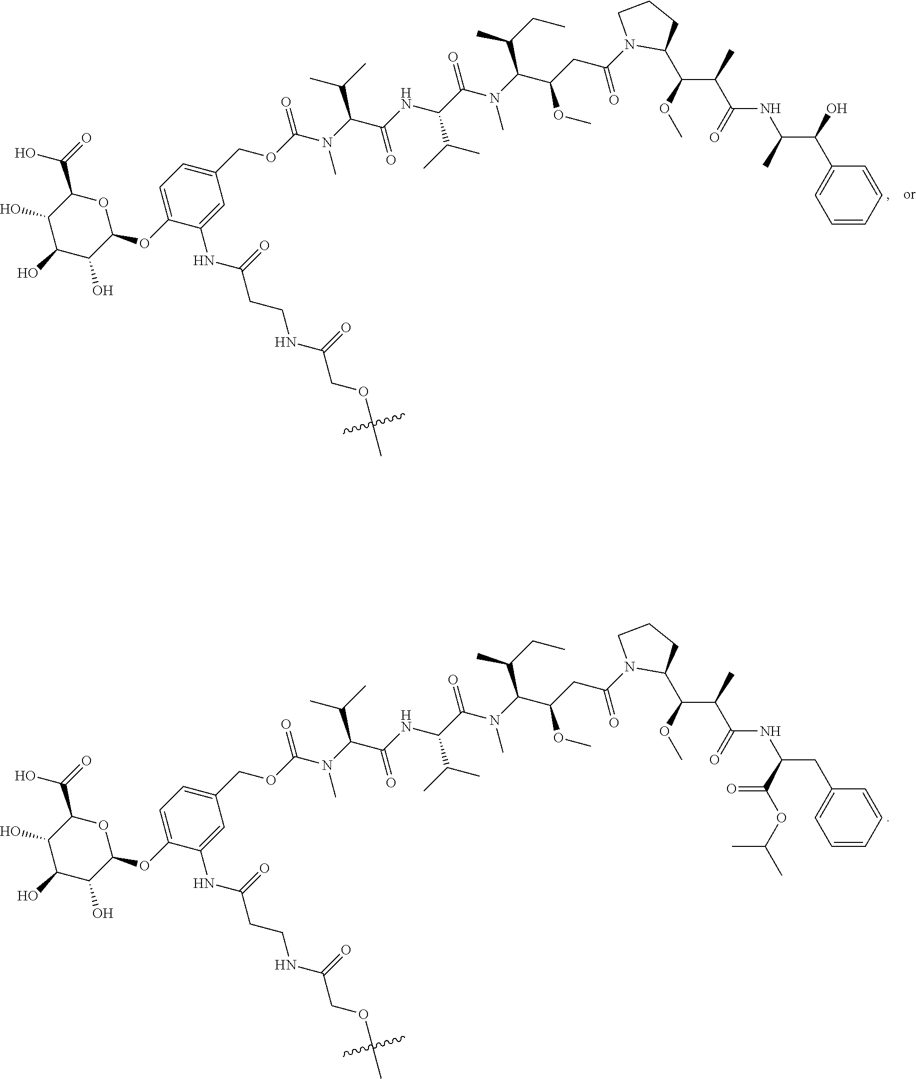

In certain embodiments, the enzymatic labile structure is liable to phosphatase and is selected from

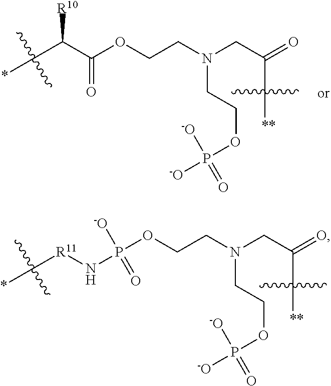

wherein * is the site covalently attached to G¹, ** is the site covalently attached to D, each of R¹⁰ and R¹¹ is independently hydrogen, aliphatic, heteroaliphatic, cycloalkyl, or heterocycloalkyl moiety.

In certain embodiments, G¹ is

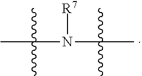

In certain embodiments, -G¹-L^{p}-D is selected from the group consisting of:

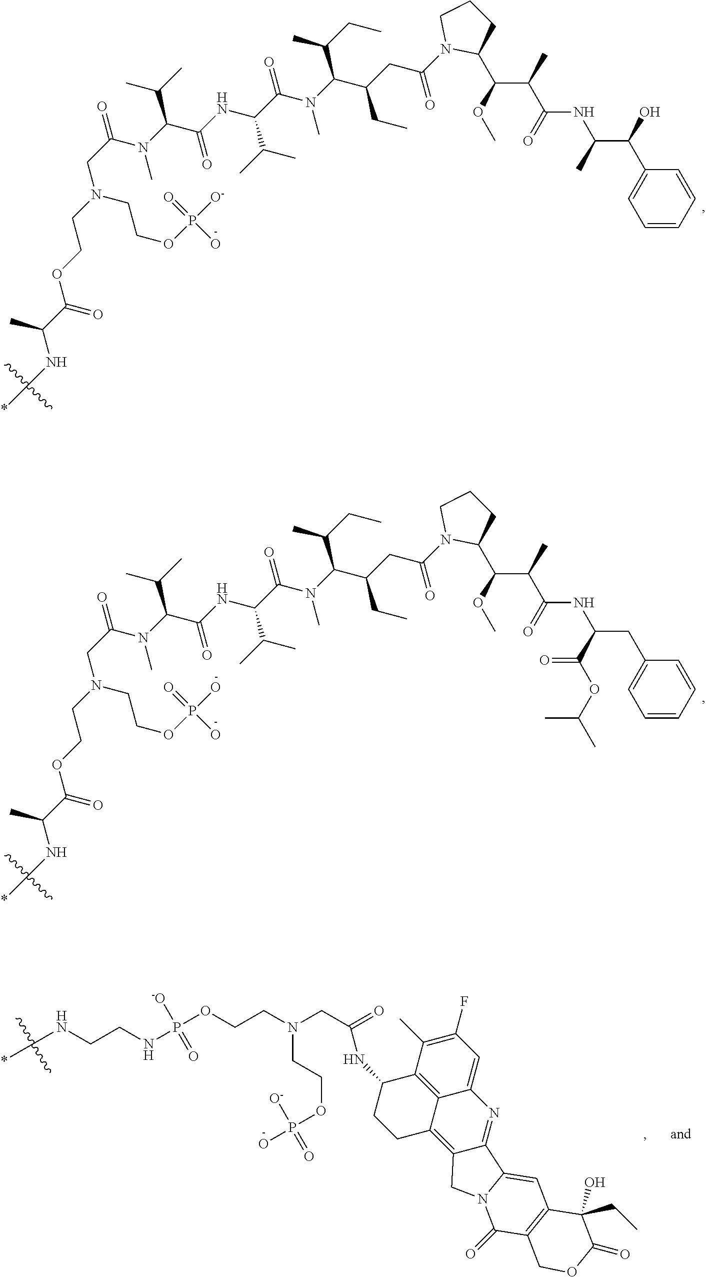

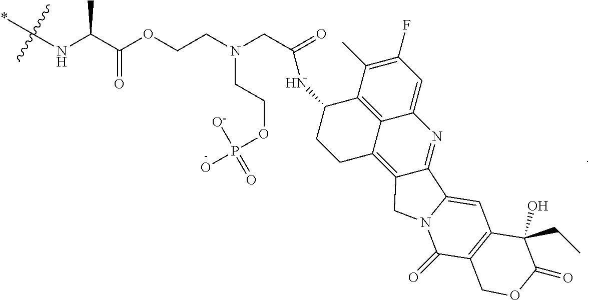

In certain embodiments, the enzymatic labile structures are liable to sulfatase and is

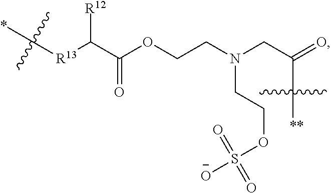

wherein * is the site covalently attached to G¹, ** is the site covalently attached to D, each of R¹² and R¹³ is independently hydrogen, aliphatic, heteroaliphatic, cycloalkyl, or heterocycloalkyl moiety.

In some embodiments, G¹ is

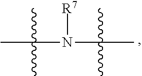

wherein R⁷ is selected from hydrogen, aliphatic, heteroaliphatic, cycloalkyl, or heterocycloalkyl moiety.

In some embodiments, G¹ is

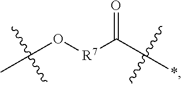

wherein * is the site covalently attached to L^{p}, R⁷ is alkyl.

In certain embodiments, -G¹-L^{p}-D is:

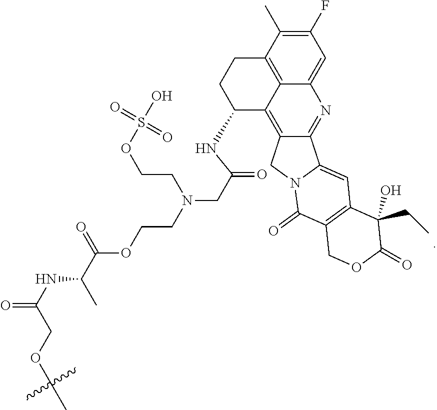 [SMILES: *OCC(=O)N[C@@H](C)C(=O)OCCN(CCOS(=O)(=O)O)CC(=O)N[C@@H]1CCc2c(C)c(F)cc3nc4c(c1c23)Cn1c-4cc2c(c1=O)COC(=O)[C@]2(O)CC]

In some embodiments, n is an integer from 1 to 100; m is an integer from 1 to 100; and p is an integer from 1 to 50.

In some embodiments, the therapeutic agent has antiproliferative activity against a target cell or pathway.

In certain embodiments, the antiproliferative activity is selected from cytostatic and/or cytotoxic activity.

In certain embodiments, the therapeutic agent is selected from anti-cancer substances, cytotoxic drugs, radionuclides, vitamins, anti-AIDS substances, antibiotics, immunosuppressants, immunomodulatory compounds, therapeutic RNAs, anti-viral substances, enzyme inhibitors, neurotoxins, opioids, hypnotics, anti-histamines, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, antisecretory factors, anticoagulants and/or antithrombotic agents, local anesthetics, ophthalmics, prostaglandins, anti-depressants, anti -psychotic substances, anti-emetics, imaging agents.

In certain embodiments, the therapeutic agent comprises amino acid-based molecules.

In certain embodiments, the amino acid-based molecules comprise peptides, polypeptides, enzymes, antibodies, immunoglobulins, or functional fragments thereof.

In certain embodiments, the therapeutic agent has a chemically reactive group.

In certain embodiments, the chemically reactive group comprises —COOH, primary amine, secondary amine-NHR, —OH, —SH, —C(O)H, C(O)R¹⁴. —C(O)NHR¹⁵, C(S)OH, —S(O)₂OR¹⁵, —P(O)₂OR¹⁵, —CN, —NC or —ONO, in which R¹⁴ is selected from aliphatic, heteroaliphatic, cycloalkyl, heterocycloalkyl, aryl or heteroaryl, and R¹⁵ is selected from a hydrogen, aliphatic, heteroaliphatic, cycloalkyl, heterocycloalkyl, aryl or heteroaryl.

In certain embodiments, G² is selected from the group consisting of:

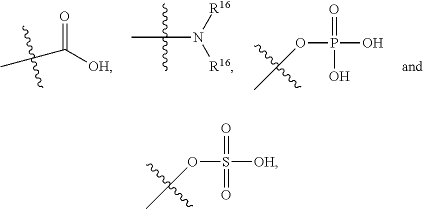

wherein each R¹⁶ is independently hydrogen, aliphatic, heteroaliphatic, cycloalkyl, heterocycloalkyl, aryl or heteroaryl.

In some embodiments, the targeting moiety is an antibody and/or fragment thereof.

In certain embodiments, the targeting moiety is an antibody IgG1, IgG2, IgG3, and IgG4.

In certain embodiments, the targeting moiety is selected from the group consisting of a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer, a multispecific antibody, a camelized single domain antibody, a nanobody, a domain antibody, or a bivalent domain antibody.

### Synthetic Methods

Any available techniques can be used to make the conjugates provided herein or compositions including them, and intermediates and components (e.g., carriers and modifiers) useful for making them. For example, semi-synthetic and fully synthetic methods may be used.

The synthetic processes of the disclosure can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, ester or prodrug thereof.

In some embodiments, the linker-polymer compound provided herein can conjugate with both a targeting moiety and a therapeutic agent (D). The linker-polymer compound provided herein comprises a linking moiety suitable for connecting a targeting moiety and a linking moiety suitable for connecting a drug (D).

In some embodiments, the conjugates provided herein are formed in several steps, including (1) modifying the polymer carrier so that the polymer carrier contains a functional group that can react with a functional group of the targeting moiety or its derivative and a functional group that can react with a functional group of the drug or its derivative; (2) reacting the modified polymer with the drug or its derivative so that the drug is linked to the modified polymer; (3) reacting the modified polymer-drug conjugate with the targeting moiety or its derivative to form the conjugate provided herein.

In another embodiment the conjugates are formed in several steps: (1) modifying the polymer carrier so that the polymer carrier contains a functional group that can react with a functional group of the targeting moiety or its derivative and a functional group that can react with a functional group of a first drug or its derivative; (2) reacting the modified polymer with the first drug or its derivative so that the first drug is linked to the modified polymer; (3) modifying the resultant polymer-drug conjugate so that it contains a different functional group that can react with a functional group of a second drug or its derivative; (4) reacting the modified polymer-drug conjugate with the second drug or its derivative so that the second drug is linked to the modified polymer-drug conjugate; (5) reacting the modified polymer-drug conjugate of step (4) with the targeting moiety or its derivative to form the conjugate provided herein.

The synthetic processes of the invention can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, ester or prodrug thereof.

Drug compounds used for the conjugates provided herein can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, incorporated by reference herein, are useful and recognized reference textbooks of organic synthesis known to those in the art.

Conjugates of the present disclosure can be conveniently prepared by a variety of methods familiar to those skilled in the art. The conjugates of the present disclosure with each of the formulae described herein may be prepared from commercially available starting materials or starting materials which can be prepared using literature procedures. The procedures show the preparation of representative conjugates of the present disclosure.

Conjugates designed, selected and/or optimized by methods described above, once produced, can be characterized using a variety of assays known to those skilled in the art to determine whether the conjugates have biological activity. For example, the conjugates can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the conjugate molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

### Pharmaceutical Composition

For the purposes of administration, in some embodiments, the conjugates provided herein are administered as a raw chemical or are formulated as pharmaceutical compositions.

Therefore, in one aspect, the present disclosure provides a pharmaceutical composition comprising one or more conjugates as disclosed herein and an acceptable carrier, such as a stabilizer, buffer, and the like. The conjugates can be administered and introduced into a subject by standard means, with or without stabilizers, buffers, and the like, to form a pharmaceutical composition. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral administration including intravenous, intraarterial, subcutaneous, intraperitoneal or intramuscular injection or infusion or intracranial, e.g., intrathecal or intraventricular, administration. The conjugates can be formulated and used as sterile solutions and/or suspensions for injectable administration; lyophilized powders for reconstitution prior to injection/infusion; topical compositions; as tablets, capsules, or elixirs for oral administration; or suppositories for rectal administration, and the other compositions known in the art.

A pharmacological composition or formulation refers to a composition or formulation in a form suitable for administration, e.g., systemic administration, into a cell or subject, including for example a human. Suitable forms, in part, depend upon the use or the route of entry, for example oral, inhaled, transdermal, or by injection/infusion. Such forms should not prevent the composition or formulation from reaching a target cell (i.e., a cell to which the drug is desirable for delivery). For example, pharmacological compositions injected into the blood stream should be soluble. Other factors are known in the art, and include considerations such as toxicity and forms that prevent the composition or formulation from exerting its effect.

As used herein, the term “systemic administration” means in vivo systemic absorption or accumulation of the modified polymer in the blood stream followed by distribution throughout the entire body. Administration routes that lead to systemic absorption include, without limitation: intravenous, subcutaneous, intraperitoneal, inhalation, oral, intrapulmonary, and intramuscular. Each of these administration routes exposes the modified polymers to an accessible diseased tissue. The rate of entry of an active agent into the circulation has been shown to be a function of molecular weight or size. The use of a conjugate provided herein can localize the drug delivery in certain cells, such as cancer cells via the specificity of targeting moieties.

As used herein, the term “pharmaceutically acceptable formulation” means a composition or formulation that allows for the effective distribution of the conjugates in the physical location most suitable for their desired activity. In some embodiments, effective delivery occurs before clearance by the reticuloendothelial system or the production of off-target binding which can result in reduced efficacy or toxicity. Non-limiting examples of agents suitable for formulation with the conjugates include: P-glycoprotein inhibitors (such as Pluronic P85), which can enhance entry of active agents into the CNS; biodegradable polymers, such as poly (DL-lactide-coglycolide) microspheres for sustained release delivery after intracerebral implantation; and loaded nanoparticles, such as those made of polybutylcyanoacrylate, which can deliver active agents across the blood brain barrier and can alter neuronal uptake mechanisms.

Also included herein are pharmaceutical compositions prepared for storage or administration, which include a pharmaceutically effective amount of the desired conjugates in a pharmaceutically acceptable carrier or diluent. Acceptable carriers, diluents, and/or excipients for therapeutic use are well known in the pharmaceutical art. For example, buffers, preservatives, bulking agents, dispersants, stabilizers, dyes, can be provided. In addition, antioxidants and suspending agents can be used Examples of suitable carriers, diluents and/or excipients include, but are not limited to: (1) Dulbecco's phosphate buffered saline, pH about 6.5, which would contain about 1 mg/ml to 25 mg/ml human serum albumin, (2) 0.9% saline (0.9% w/v NaCl), and (3) 5% (w/v) dextrose.

As used herein, the term “pharmaceutically effective amount” refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Pharmaceutically effective amounts for a given situation can be determined by routine experimentation that is within the skill and judgment of the clinician.

For any conjugate, the pharmaceutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The dosage may vary within this range depending upon the dosage form employed, sensitivity of the patient, and the route of administration.

For example, linker-polymer-drug compound or targeting moiety-linker-polymer-drug conjugates can be evaluated for their ability to inhibit tumor growth in several cell lines using Cell titer Glo. Dose response curves can be generated using SoftMax Pro software and IC₅₀ values can be determined from four-parameter curve fitting. Cell lines employed can include those which are the targets of the targeting moiety and a control cell line that is not the target of the targeting moiety contained in the test conjugates.

In some embodiments, the conjugates are formulated for parenteral administration by injection including using conventional catheterization techniques or infusion. Formulations for injection may be presented in unit dosage form, e.g., in ampules or in multi-dose containers, with an added preservative. The conjugates can be administered parenterally in a sterile medium. The conjugate, depending on the vehicle and concentration used, can either be suspended or dissolved in the vehicle. Advantageously, adjuvants such as local anesthetics, preservatives, and buffering agents can be dissolved in the vehicle. The term “parenteral” as used herein includes percutaneous, subcutaneous, intravascular (e.g., intravenous), intramuscular, or intrathecal injection or infusion techniques and the like. In addition, there is provided a pharmaceutical formulation comprising conjugates and a pharmaceutically acceptable carrier. One or more of the conjugates can be present in association with one or more non-toxic pharmaceutically acceptable carriers and/or diluents and/or adjuvants, and if desired other active ingredients.

The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, a bland fixed oil can be employed including synthetic mono- or diglycerides. In addition, fatty acids such as oleic acid find use in the preparation of injectables.

The conjugates and compositions described herein may be administered in appropriate form, preferably parenterally, more preferably intravenously. For parenteral administration, the conjugates or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants.

Dosage levels of the order of from between about 0.001 mg and about 140 mg per kilogram of body weight per day are useful in the treatment of the above-indicated conditions (between about 0.05 mg and about 7 g per subject per day). In some embodiments, the dosage administered to a patient is between about 0.001 mg/kg to about 100 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. The amount of conjugate that can be combined with the carrier materials to produce a single dosage form varies depending upon the host treated and the particular mode of administration. Dosage unit forms can generally contain from between about 0.001 mg and about 100 mg; between about 0.01 mg and about 75 mg; or between about 0.01 mg and about 50 mg; or between about 0.01 mg and about 25 mg; of a conjugate.

For intravenous administration, the dosage levels can comprise ranges described above, or from about 0.01 to about 200 mg of a conjugate per kg of the animal's body weight. In some embodiments, the composition can include from about 1 to about 100 mg of a conjugate per kg of the animal's body weight. In some embodiments, the amount administered will be in the range from about 0.1 to about 25 mg/kg of body weight of a compound.

In some embodiments, the conjugates can be administered are as follows. The conjugates can be given daily for about 5 days either as an i.v., bolus each day for about 5 days, or as a continuous infusion for about 5 days.

Alternatively, the conjugates can be administered once a week for six weeks or longer. As another alternative, the conjugates can be administered once every two or three weeks. Bolus doses are given in about 50 to about 400 ml of normal saline to which about 5 to about 10 ml of human serum albumin can be added. Continuous infusions are given in about 250 to about 500 ml of normal saline, to which about 25 to about 50 ml of human serum albumin can be added, per 24 hour period.

In some embodiments, about one to about four weeks after treatment, the patient can receive a second course of treatment. Specific clinical protocols with regard to route of administration, excipients, diluents, dosages, and times can be determined by the skilled artisan as the clinical situation warrants.

In other embodiments, the therapeutically effective amount may be provided on another regular schedule, i.e., daily, weekly, monthly, or yearly basis or on an irregular schedule with varying administration days, weeks, months, etc. Alternatively, the therapeutically effective amount to be administered may vary. In some embodiments, the therapeutically effective amount for the first dose is higher than the therapeutically effective amount for one or more of the subsequent doses. In some embodiments, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the recommendations of the relevant regulatory bodies and judgment of a health-care practitioner. The therapeutically effective amounts described herein refer to total amounts administered for a given time period; that is, if more than one different conjugate described herein is administered, the therapeutically effective amounts correspond to the total amount administered. It is understood that the specific dose level for a particular subject depends upon a variety of factors including the activity of the specific conjugate, the age, body weight, general health, sex, diet, time of administration, route of administration, and rate of excretion, combination with other active agents, and the severity of the particular disease undergoing therapy.

For administration to non-human animals, the conjugates can also be added to the animal feed or drinking water. It can be convenient to formulate the animal feed and drinking water so that the animal takes in a therapeutically appropriate quantity of the conjugates along with its diet. It can also be convenient to present the conjugates as a premix for addition to the feed or drinking water.

The conjugates can also be administered to a subject in combination with other therapeutic compounds to increase the overall therapeutic effect. The use of multiple compounds to treat an indication can increase the beneficial effects while reducing the presence of side effects. In some embodiments, the conjugates are used in combination with chemotherapeutic agents, such as those disclosed in U.S. Pat. No. 7,303,749. In other embodiments the chemotherapeutic agents, include, but are not limited to letrozole, oxaliplatin, docetaxel, 5-FU, lapatinib, capecitabine, leucovorin, erlotinib, pertuzumab, bevacizumab, and gemcitabine. The present disclosure also provides pharmaceutical kits comprising one or more containers filled with one or more of the conjugates and/or compositions of the present disclosure, including, one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products. The compositions described herein can be packaged as a single dose or for continuous or periodic discontinuous administration. For continuous administration, a package or kit can include the conjugates in each dosage unit (e.g., solution or other unit described above or utilized in drug delivery), and optionally instructions for administering the doses daily, weekly, or monthly, for a predetermined length of time or as prescribed. If varying concentrations of a composition, of the components of the composition, or the relative ratios of the conjugates or agents within a composition over time is desired, a package or kit may contain a sequence of dosage units which provide the desired variability.

A number of packages or kits are known in the art for dispensing pharmaceutical agents for periodic oral use. In some embodiments, the package has indicators for each period. In some embodiments, the package is a labeled blister package, dial dispenser package, or bottle. The packaging means of a kit may itself be geared for administration, such as a syringe, pipette, eye dropper, or other such apparatus, from which the formulation may be applied to an affected area of the body, injected into a subject, or even applied to and mixed with the other components of the kit.

### Method of Treatment of Disease

The targeting moiety-linker-polymer-drug conjugate provided herein can be used in methods of treating animals (for example mammals, such as humans and includes males, females, infants, children and adults).

In some embodiments, the conjugates provided herein may be used in a method of treating animals which comprises administering to the animal the conjugate of the present disclosure. The conjugates of this invention can be used as drug carriers and drug carrier components, in systems of controlled drug release, preparations for low-invasive surgical procedures, etc. Pharmaceutical formulations can be injectable, implantable, etc.

In yet another aspect, the present disclosure provides a method of treating a disease or disorder in a subject in need thereof, comprising administering to the subject an efficient amount of at least one conjugate provided herein, wherein said conjugate releases one or more therapeutic agents upon biodegradation.

In some embodiments, the conjugates provided herein can be administered in vitro, in vivo and/or ex vivo to treat subjects and/or to modulate the growth of selected cell populations including, for example, cancer. In some embodiments, the particular types of cancers that can be treated with the conjugates provided herein include, but are not limited to: (1) solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor, cervical cancer, uterine cancer, testicular cancer, small cell lung carcinoma, non-small cell lung carcinoma, bladder carcinoma, lung cancer, epithelial carcinoma, glioma, glioblastoma, multiforme astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; (2) blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, e.g., lymphoblastic myelogenous and lymphocytic myelocytic leukemias; and (3) lymphomas such as Hodgkin's disease, non-Hodgkin's Lymphoma, Multiple myeloma, Waldenstrom's macroglobulinemia, Heavy chain disease, and Polycythemia vera.

In some embodiments, the conjugates provided herein can be administered in vitro, in vivo and/or ex vivo to treat subjects and/or to modulate the growth of selected cell populations in patients having anal, astrocytoma, leukemia, lymphoma, head and neck, liver, testicular, cervical, sarcoma, hemangioma, esophageal, eye, laryngeal, mouth, mesothelioma, skin, myeloma, oral, rectal, throat, bladder, breast, uterus, ovary, prostate, lung, colon, pancreas, renal, or gastric cancer.

In certain embodiment, the cancers are selected from the group consisting of breast cancer, gastric cancer, non-small cell lung cancer (NSCLC), and ovarian cancer.

In some embodiment, the conjugates provided herein can be administered in vitro, in vivo and/or ex vivo to treat, prevent, reduce the risk of developing and/or delay onset of certain pathologies, for example, a cancer. For example, the conjugates provided herein are useful in treating, preventing, delaying the progression of or otherwise ameliorating a symptom of a cancer selected from the group consisting of anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, skin cancer, myeloma, oral cancer, rectal cancer, throat cancer, bladder cancer, breast cancer, uterine cancer, ovarian cancer, prostate cancer, lung cancer, non-small cell lung cancer (NSCLC), colon cancer, pancreatic cancer, renal cancer, and gastric cancer.

In some embodiments the conjugates provided herein can be administered in vitro, in vivo and/or ex vivo to treat autoimmune diseases, such as systemic lupus, rheumatoid arthritis, psoriasis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, and AIDS; and parasite infections, such as giardiasis, amoebiasis, schistosomiasis, and the like.

In some embodiments, the conjugates provided herein can also be used for the manufacture of a medicament useful for treating or lessening the severity of disorders, such as, characterized by abnormal growth of cells (e.g., cancer).

In some embodiments, the therapeutic agent is locally delivered to a specific target cell, tissue, or organ.

In certain embodiments, the conjugates provided herein can further comprise or are associated with a diagnostic label. In certain embodiments, the diagnostic label is selected from the group consisting of: radiopharmaceutical or radioactive isotopes for gamma scintigraphy and PET, contrast agent for Magnetic Resonance Imaging (MRI), contrast agent for computed tomography, contrast agent for X-ray imaging method, agent for ultrasound diagnostic method, agent for neutron activation, moiety which can reflect, scatter or affect X-rays, ultrasounds, radiowaves and microwaves and fluorophores. In certain exemplary embodiments, the conjugate is further monitored in vivo.

Examples of diagnostic labels include, but are not limited to, diagnostic radiopharmaceutical or radioactive isotopes for gamma scintigraphy and PET, contrast agent for Magnetic Resonance Imaging (MRI) (for example paramagnetic atoms and superparamagnetic nanocrystals), contrast agent for computed tomography, contrast agent for X-ray imaging method, agent for ultrasound diagnostic method, agent for neutron activation, and moiety which can reflect, scatter or affect X-rays, ultrasounds, radiowaves and microwaves, fluorophores in various optical procedures, etc. Diagnostic radiopharmaceuticals include y-emitting radionuclides, e.g., indium-111, technetium-99m and iodine-131, etc. Contrast agents for MRI (Magnetic Resonance Imaging) include magnetic compounds, e.g., paramagnetic ions, iron, manganese, gadolinium, lanthanides, organic paramagnetic moieties and superparamagnetic, ferromagnetic and antiferromagnetic compounds, e.g., iron oxide colloids, ferrite colloids, etc. Contrast agents for computed tomography and other X-ray based imaging methods include compounds absorbing X-rays, e.g., iodine, barium, etc. Contrast agents for ultrasound based methods include compounds which can absorb, reflect and scatter ultrasound waves, e.g., emulsions, crystals, gas bubbles, etc. Still other examples include substances useful for neutron activation, such as boron and gadolinium. Further, labels can be employed which can reflect, refract, scatter, or otherwise affect X-rays, ultrasound, radiowaves, microwaves and other rays useful in diagnostic procedures. Fluorescent labels can be used for photoimaging. In certain embodiments a modifier comprises a paramagnetic ion or group.

In another aspect, the present disclosure provides a method of treating a disease or disorder in a subject, comprising preparing an aqueous formulation of at least one conjugate provided herein and parenterally injecting said formulation in the subject.

In another aspect, the present disclosure provides a method of treating a disease or disorder in a subject, comprising preparing an implant comprising at least one conjugate provided herein, and implanting said implant into the subject. In certain embodiments, the implant is a biodegradable gel matrix.

In another aspect, the present disclosure provides a method for treating of an animal in need thereof, comprising administering a conjugate according to the methods described above.

In another aspect, the present disclosure provides a method for eliciting an immune response in an animal, comprising administering a conjugate as in the methods described above.

In another aspect, the present disclosure provides a method of diagnosing a disease in an animal, comprising steps of: administering a conjugate as in the methods described above, wherein said conjugate comprises a detectable molecule; and detecting the detectable molecule.

In some embodiments, the step of detecting the detectable molecule is performed non-invasively. In some embodiments, the step of detecting the detectable molecule is performed using suitable imaging equipment.

In some embodiments, a method for treating an animal comprises administering to the animal the conjugates provided herein as a packing for a surgical wound from which a tumor or growth has been removed. The conjugate packing will replace the tumor site during recovery and degrade and dissipate as the wound heals.

In certain embodiments, the conjugate provided herein is associated with a diagnostic label for in vivo monitoring.

The conjugates provided herein can be used for therapeutic, preventative, and analytical (diagnostic) treatment of animals. The conjugates are intended, generally, for parenteral administration, but in some cases may be administered by other routes.

In some embodiments, soluble or colloidal conjugates are administered intravenously. In some embodiments, soluble or colloidal conjugates are administered via local (e.g., subcutaneous, intramuscular) injection. In some embodiments, solid conjugates (e.g., particles, implants, drug delivery systems) are administered via implantation or injection.

In some embodiments, conjugates comprising a detectable label are administered to study the patterns and dynamics of label distribution in animal body.

In certain embodiments, any one or more of the conjugates provided herein may be used in practicing any of the methods described above.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same.

### Examples

For the purpose of illustration, the following examples are included. The Examples provided herein describe the synthesis of the compounds and conjugates disclosed herein as well as intermediates used to prepare the compounds and conjugates. However, it is to be understood that these examples do not limit the present disclosure and are only meant to suggest a method of practicing the present disclosure. Persons skilled in the art will recognize that the chemical reactions described may be readily adapted to prepare a number of other compounds or conjugates of the present disclosure, and alternative methods for preparing the compounds and conjugates of the present disclosure are deemed to be within the scope of the present disclosure. Besides, persons skilled in the art will also understand that individual steps described herein or in the separate batches of a compound may be combined. Alternatively, other reactions disclosed herein or known in the art will be recognized as having applicability for preparing other compounds of the present disclosure. The following description is, therefore, not intended to limit the scope of the present disclosure, but rather is specified by the claims appended hereto.

### Abbreviations

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.

| | |
|---|---|
| DMSO | Dimethyl sulfoxide |
| DMF | N,N-Dimethylformamide |
| HATU | O-(7-Azabenzotriazol-1-yl)-N,N,N,N-tetramethyl uronium |
| | hexafluorophosphate |
| THF | Tetrahydrofuran |
| DIPEA | N,N-Diisopropylethylamine |
| IPA | Isopropanol |
| DCM | Dichloromethane |
| EDCI | N-(3-dimethylaminopropyl)-n″-ethylcarbodiimidehydrochloride |
| DMAP | 4-Dimethylaminopyridine |
| TFA | Trifluoroacetic acid |
| EEDQ | 2-Ethoxy-1-ethoxycarbonyl-1,2-dihydroquinoline |
| HOBt | N-Hydroxybenzotrizole |
| TBAF | Tetra-n-butylammonium fluoride |
| EGE | Ethoxyethyl glycidyl ether |
| t-BGA | t-Butyl glycidoxy acetate |
| EA | Ethyl acetate |
| PS | Polystyrene |
| NHS | N-Hydroxysuccinimide |
| PEG | Polyethylene glycol |
| PB | Phosphate Buffer |
| TCEP | Tris(2-carboxyethyl)phosphine |
| EDTA | Ethylenediaminetetraacetic acid |

### Example 1. Synthesis of the Copolymer Linker Compound 1

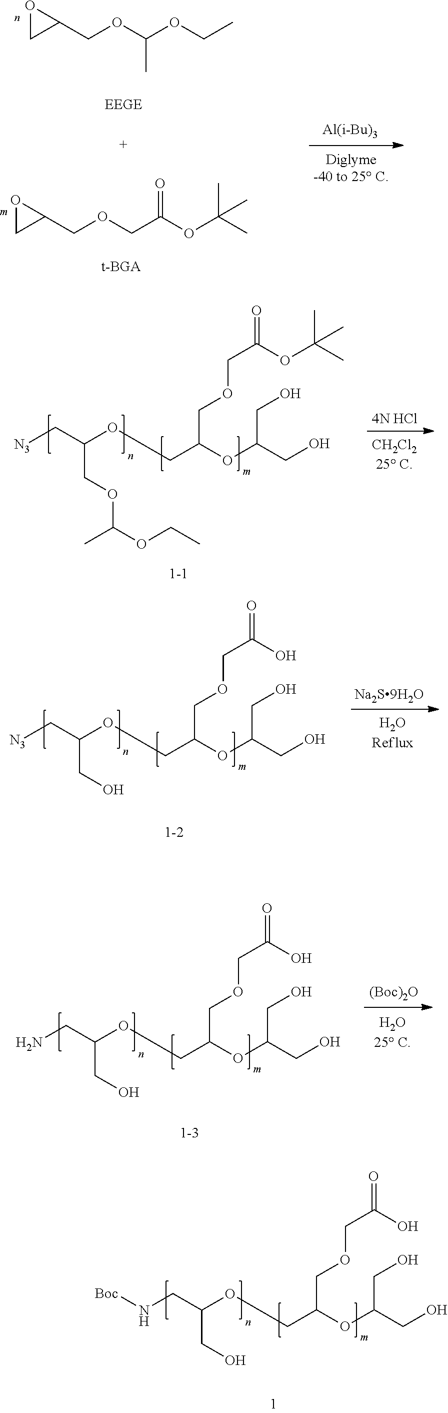

To the solution of N₃NBu₄ (1.98 g, 6.98 mmol) in diglyme (200 mL), EGE (61 g, 418.6 mmol) and t-BGA (39.3 g, 209.3 mmol) were added under N₂. The mixture was cooled to −40° C., then Al(iBu)₃ (30 ml, 29.7 mmol, 1M in hexanes) was added slowly. The reaction was warmed to room temperature and stirred under N₂ for 16 hours. The reaction was quenched with EtOH, diluted with water and extracted with EA. The combined organic phase was washed with brine, dried with Na₂SO₄, filtered and concentrated under reduced pressure to afford compound 1-1 (103 g) as colorless oil with Mw 27,000 which was determined by GPC with PS as standard.

To the solution of compound 1-1 (50 g, 1.85 mmol) in DCM (500 mL), HCl (200 mL, 1 M in 1,4-dioxane) was added slowly and the reaction mixture was stirred at room temperature for 16 hours. After completion, the solvent was removed under reduced pressure and the residue was dissolved with 10% NaOH solution. After adjusted the pH to 7 with 4 N HCl, the total volume of the solution was adjusted to 500 mL with water to obtain compound 1-2 as water solution.

To the solution of 1-2, Na₂S·9H₂O (30 g, 125 mmol) was added and reaction mixture was heated at 100° C. for 18 hours. After completion, the reaction was cooled to room temperature and the pH was adjust to 10 with 4 N HCl. The mixture was dialyzed with 3 KD MWCO to afford compound 1-3 as water solution (300 mL).

To the solution of 1-3, NaOH (5 g, 125 mmol) and (Boc)₂O (30 g, 125 mmol) were added and the reaction mixture was stirred at room temperature for 18 hours. After completion, the mixture was dialyzed with 3 KD MWCO to afford compound 1 as water solution (800 mL, 25 mg/mL).

1H NMR (400 MHz, D₂O) δ 3.95 (s, 100H), 3.87-3.57 (br, 800H), 1.43 (s, 9H).

### Example 2. Synthesis of Compound 2

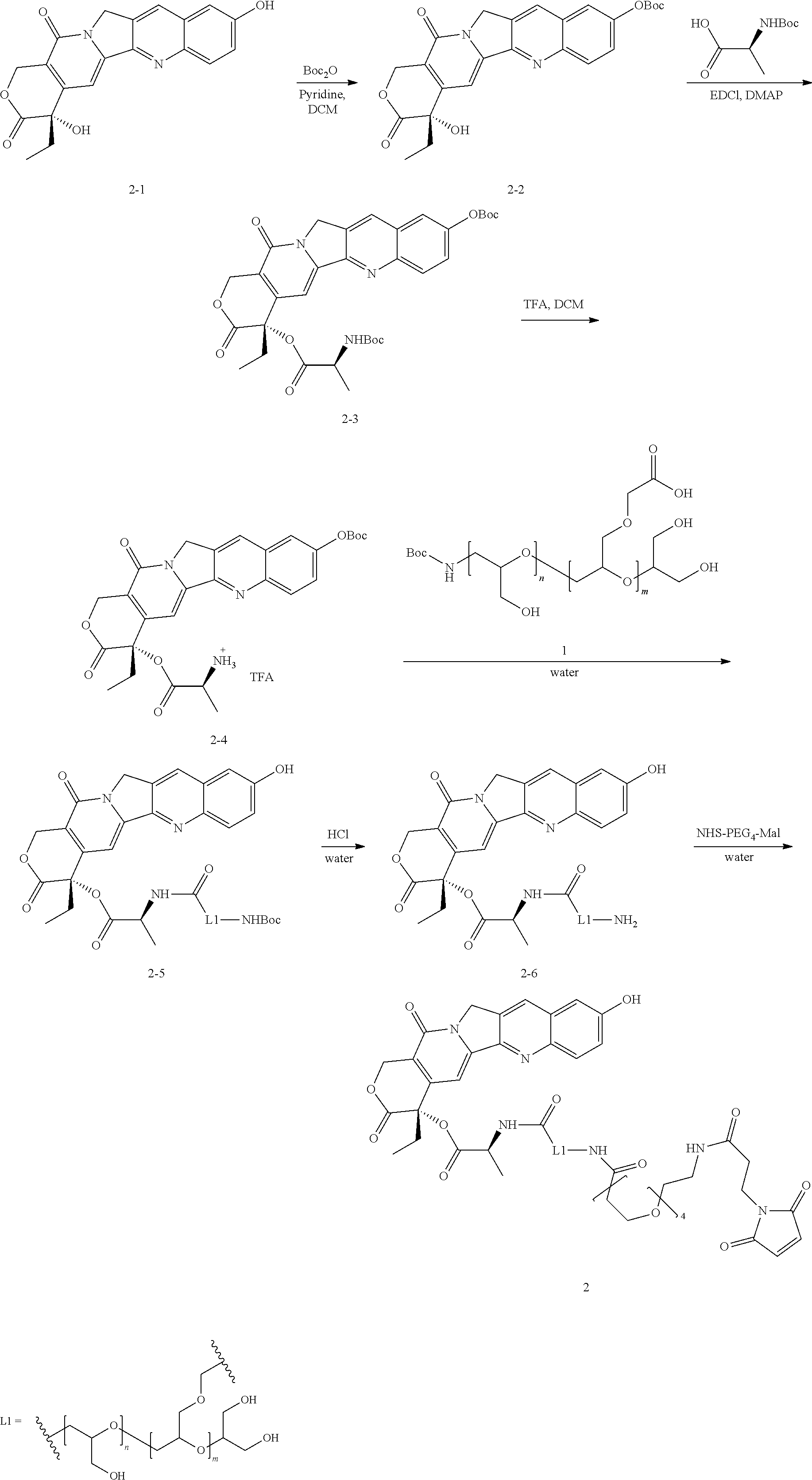

To a solution of 2-1 in THF (200 mL) was added pyridine (12 mL, 148 mmol) and Boc₂O (1.44 g, 6.59 mmol). The reaction mixture was stirred at 15° C. for 18 hours. The reaction mixture was washed with 0.5 N HCl (3×100 mL) and saturated NaHCO₃ (100 mL). The organic phase was dried over MgSO₄, filtered and concentrated in vacuo to give the desired product 2-2 (2.45 g) as a white solid.

To a solution of 2-2 (2.5 g, 5.38 mmol) in DCM (50 mL) was added (tert-butoxycarbonyl)-L-alanine (2.5 g, 13.2 mmol), EDCI (1.99 g, 10.4 mmol) and DMAP (197.3 mg, 1.61 mmol). The reaction mixture was stirred at 15° C. for 16 hours. The reaction mixture was washed with 0.5% NaHCO₃ (2×400 mL), water (400 mL), and 0.1 N HCl (2×400 mL). The organic phase was dried over anhydrous MgSO₄, filtered, and evaporated under vacuum. The residue was purified by silica gel column chromatography to give the desired product 2-3 (3.0 g, 87.7% yield) as a white solid.

To a solution of 2-3 (1.5 g, 2.36 mmol) in DCM (30 mL) was added TFA (10 mL). After stirred at 15° C. for 16 h, the solvent was removed under vacuum and the residue was purified by prep-HPLC (TFA) to give the desired product 2-4 (627 mg) as a yellow solid.

To the water solution of compound 1, the DMF solution of NHS (0.12 mg, 1.05 μmol), EDC (20 mg, 105.4 μmol) and 2-4 (23.4 mg, 42.2 μmol) was added slowly at 0° C. and the reaction mixture was stirred at room temperature for 16 hours. After completion, the mixture was dialyzed with 5 KD MWCO to afford compound 2-5 as water solution (25 mL, 5 mg/mL).

To the water solution of compound 2-5 (25 mL, 5 mg/mL), 4N HCl (1.6 mL) was added slowly at 0° C., the reaction mixture was stirred at room temperature for 16 hours. After completion, the pH of the mixture was adjusted to 7 with saturated NaHCO_{3(q)} to afford compound 2-6 which was used directly to the next step.

To the water solution of compound 2-6, NHS-PEG₄-Mal (32.6 mg, 5.25 μmol) in DMF (10 mL) was added slowly at 0° C., the reaction mixture was stirred at room temperature for 16 hours. After completion, the mixture was dialyzed with 5 KD MWCO to afford compound 2 as water solution (15 mL, 5 mg/mL).

### Example 3. Synthesis of Compound 3

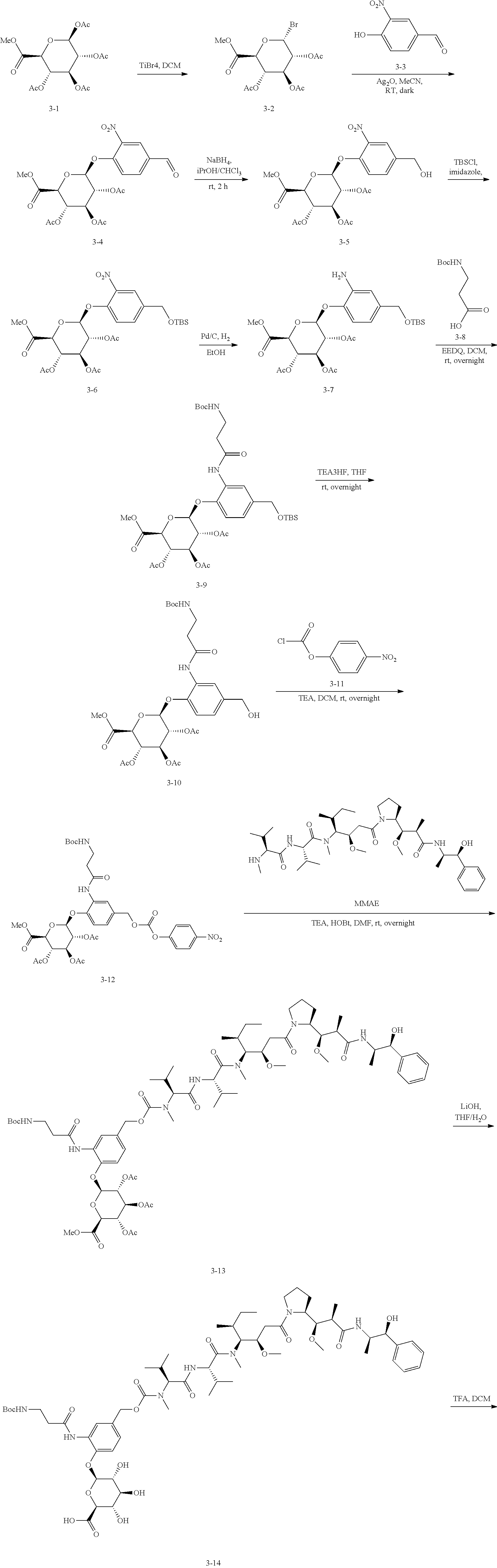

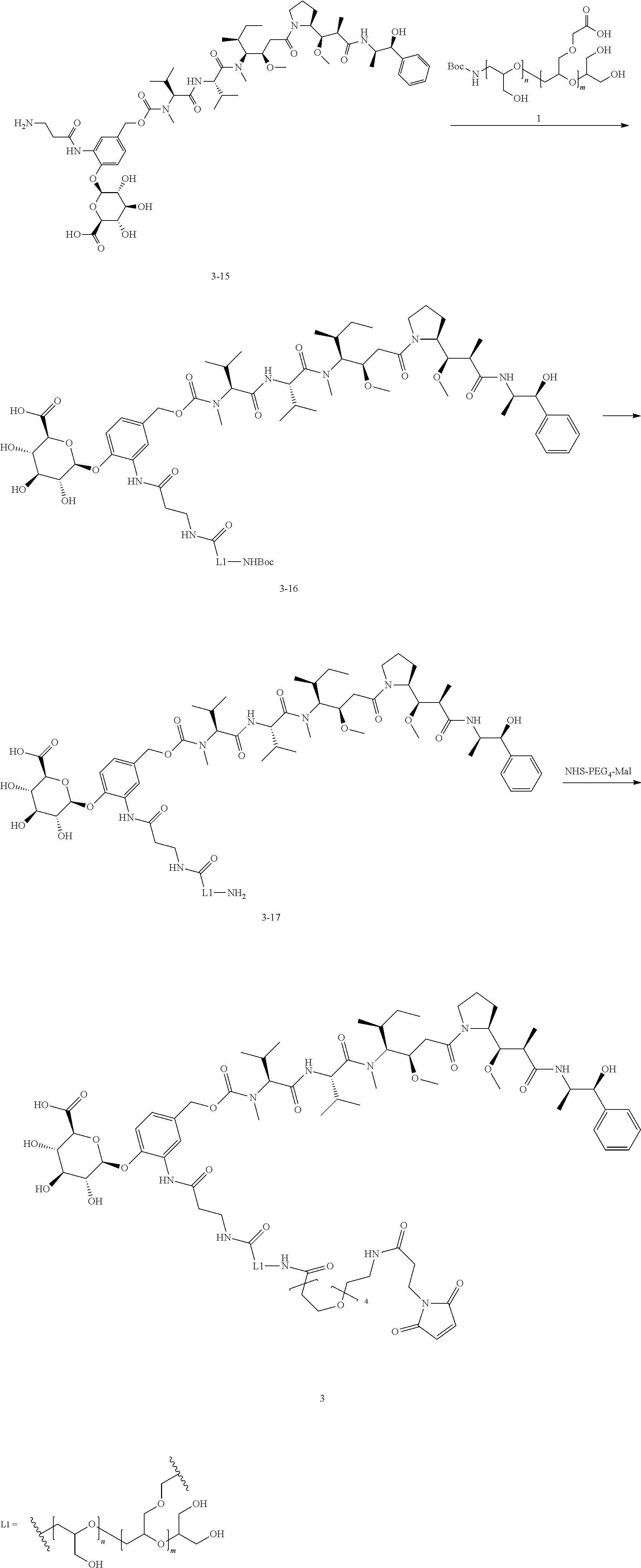

To a solution of 3-1 (10 g, 26.6 mmol) in DCM (100 mL) was added TiBr₄ (10.7 g, 29.2 mmol) under N₂. The reaction mixture was stirred at room temperature for 15 hours. The mixture was filtered and the filtrate was concentrated, the residue was purified with silica gel column chromatograph with Petroleum Ether/EtOAc=10:1 to give the compound 3-2 (8.0 g, 20.1 mmol, 75.7% yield) as a white solid.

To a solution of 3-2 (8.0 g, 20.1 mmol) in MeCN (160 mL) was added 3-3 (3.39 g, 20.1 mmol) and Ag₂O (5.12 g, 22.1 mmol) under N₂. After stirred at room temperature under dark for 15 hours, the mixture was filtered and the filtrate was concentrated under reduced pressure. The residue was purified with silica gel column chromatograph with Petroleum Ether/DCM=10:1 to give compound 3-4 (4.8 g, 9.94 mmol, 49.4% yield) as a white solid.

To the mixture of 3-4 (4.8 g, 9.94 mmol) and silica gel (2.12 g) in CHCl₃ (50 mL) and iPrOH (10 mL) was added NaBH₄ (564 mg, 14.91 mmol) under N₂ at 0° C. After stirred at 25° C. for 1.5 hour, the mixture was filtered and the solvent was evaporated to give the product 3-5 (5.0 g, 10.3 mmol, crude) as colorless oil.

To a solution of 3-5 (5.0 g, 10.3 mmol) in DMF (50 mL) was added imidazole (5.2 g, 9.26 mmol) and DMAP (282 mg, 2.31 mmol) under N₂, followed by the addition of the solution of TBSCl (2.33 g, 15.45 mmol) in DMF (30 mL). After stirred at room temperature for 16 hours, the reaction mixture was diluted with EtOAc (100 mL), washed with NH₄Cl (3×50 mL) and brine (3×50 mL). The combined organic layers were dried over Na₂SO₄, filtered and the solvent was removed in vacuo. The crude product was purified by silica gel column chromatograph (PE:EA=4:1) to give 3-6 (6.2 g, 10.3 mmol, 100% yield) as a white solid.

To a solution of 3-6 (4.2 g, 7 mmol) in EtOH (50 mL) was added 10% Pd/C (420 mg) at room temperature. The mixture was stirred under H2 (1 atm) at 25° C. for 16 hours. After completion, the mixture was filtered through Celite and washed with EtOH (50 mL). The filtrate was concentrated under vacuum to give the desired product 3-7 (3.8 g, 6.67 mmol, 95% yield) as a white solid.

To a solution of 3-7 (3.8 g, 6.67 mmol) and 3-8 (1.39 g, 7.3 mmol) in DCM (50 ml) was added EEDQ (6.6 g, 26.68 mmol). After stirred under an atmosphere N₂ at room temperature for 16 hours, the reaction mixture was diluted with water (100 mL) and extracted with DCM (3×100 mL). The organic phase was washed with brine (100 mL), dried over Na₂SO₄ and concentrated under vacuum to give the crude, which was purified by flash chromatography to give the product 3-9 (2.4 g, 3.24 mmol, 48.6% yield) as a white solid.

To a solution of 3-9 (2.4 g, 3.24 mmol) in THF (20 mL) was added TEA 3HF (2 mL). The reaction mixture was stirred at room temperature for 1.5 h. After completion, the solvent was removed and the residue was purified by silica gel column chromatography to give the desired product 3-10 (1.75 g, 2.79 mmol, 86% yield) as a white solid.

To a solution of 3-10 (1.1 g, 1.76 mmol) and TEA (356 mg, 3.52 mmol) in DCM (50 mL) was added 3-11 (532 mg, 2.64 mmol) under N₂. After was stirred at 25° C. for 16 hours, the solvent was removed and the residue was purified by silica gel column chromatography to give the desired product 3-12 (800 mg, 1.01 mmol, 57% yield) as a white solid.

To a solution of 3-12 (200 mg, 0.25 mmol) in DMF (5 mL) was added (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (181 mg, 0.25 mmol), HOBT (6.7 mg, 0.025 mmol) and TEA (50.6 mg, 0.5 mmol) under N₂. After stirred at 25° C. for 16 hours, the mixture was diluted with water and extracted with EtOAc (20 mL×3). The combined organic layer was washed with brine (20 mL×2), dried over Na₂SO₄, filtered and concentrated in vacuo to give the crude, which was purified by pre-HPLC to give the desired product 3-13 (202 mg, 0.147 mmol, 59% yield).

To a solution of 3-13 (400 mg, 0.29 mmol) in THF (10 mL) and water (2 mL) was added LiOH (27.7 mg, 1.16 mmol) at 0′T. After stirred for 4 hours at 25° C., the reaction was diluted with water (50 mL) and extracted with EtOAc (50 mL×3). The combined organic layer was washed with brine (20 mL), dried over Na₂SO₄, filtered and concentrated in vacuum to give 3-14 (300 mg, 0.24 mmol, 84% yield) as colorless oil.

To a solution of 3-14 (300 mg, 0.24 mmol) in DCM (3 mL) was added TFA (1 mL) at room temperature. After stirred at room temperature for 16 hours, the reaction was concentrated in vacuo to give the crude, which was purified by prep-HPLC (TFA) to give the desired product 3-15 (120 mg, 0.106 mmol, 44% yield).

To the water solution of compound 1 (150 mg, 6 mL, 25 mg/mL), the DMF solution of NHS (0.12 mg, 1.05 μmol), EDCI (20 mg, 105.4 μmol) and 3-15 (23.4 mg, 42.2 μmol) were added slowly at 0° C., the reaction solution was stirred at room temperature for 16 hours. After completion, the mixture was dialyzed with 5 KD MWCO to afford compound 3-16 as water solution (25 mL, 5 mg/mL).

To the water solution of compound 3-16 (25 mL, 5 mg/mL), 4N HCl (1.6 mL) was added slowly at 0° C., the reaction mixture was stirred at room temperature for 16 hours. After completion, the pH of the mixture was adjusted to 7 with saturated NaHCO_{3(aq)} to afford the solution compound 3-17 which was used directly to the next step.

To the water solution of compound 3-17, NHS-PEG₄-Mal (32.6 mg, 5.25 μmol) in DMF (10 mL) was added slowly at 0° C., the reaction mixture was stirred at room temperature for 16 hours. After completion, the mixture was dialyzed with 5 KD MWCO to afford compound 3 as water solution (19 mL, 5 mg/mL).

### Example 4. Synthesis of Compound 4

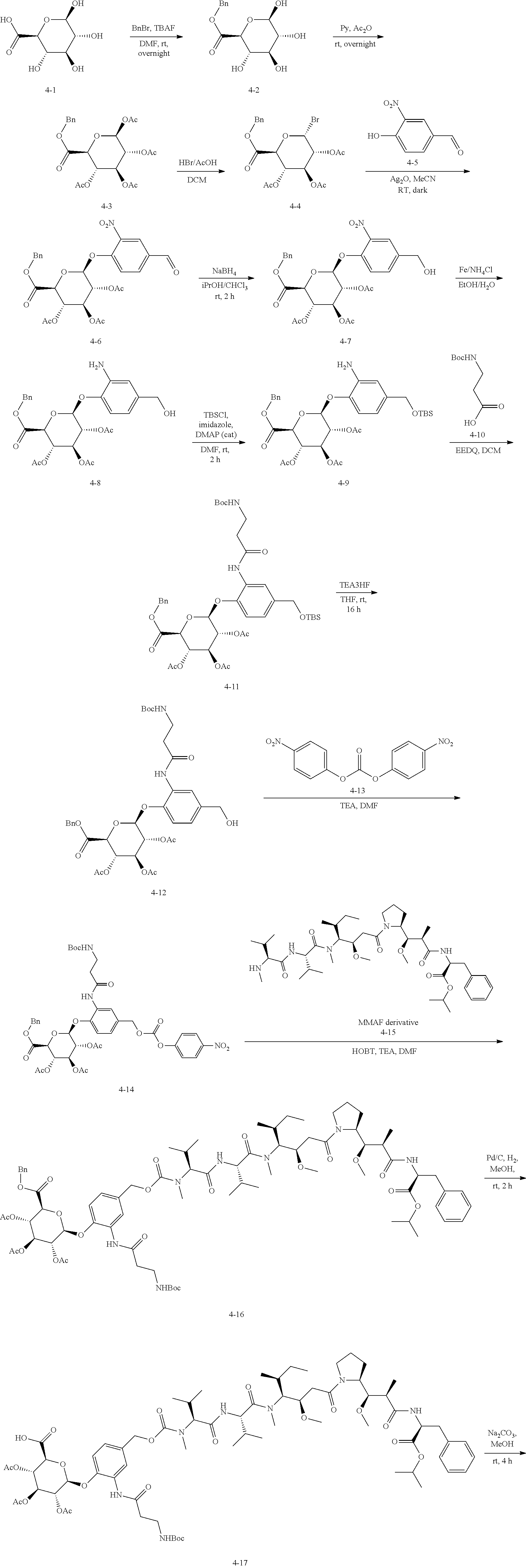

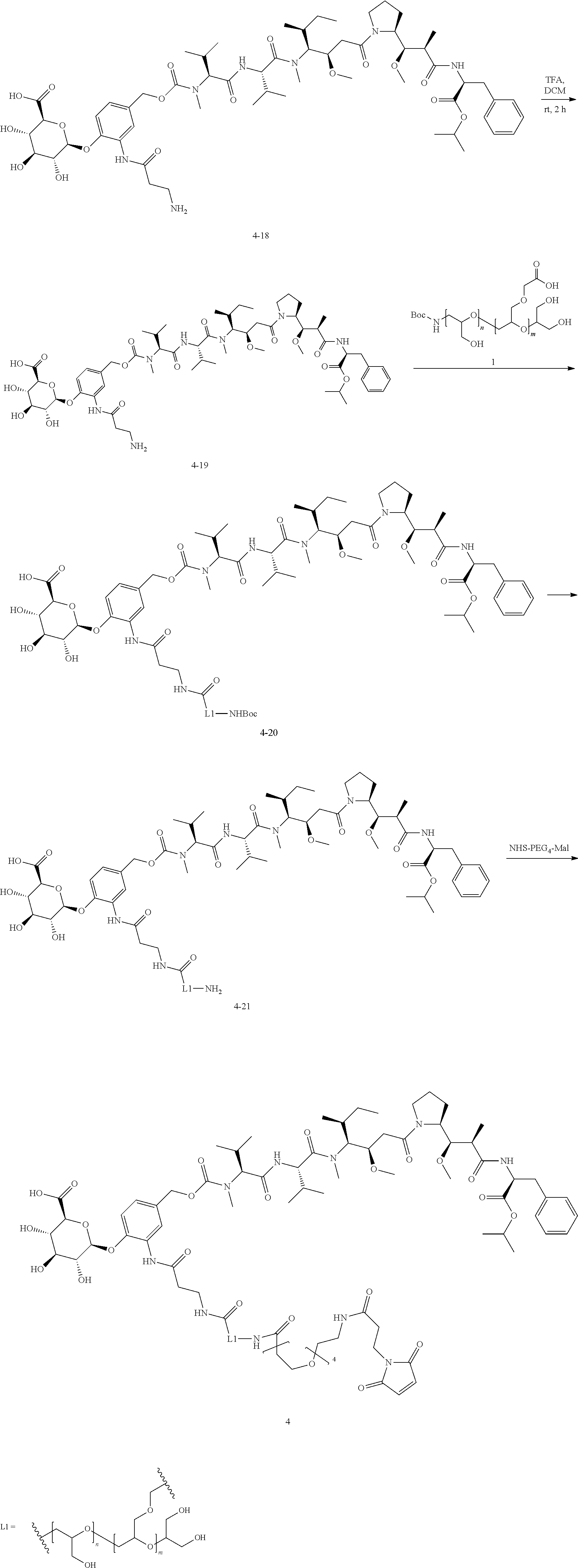

To a solution of 4-1 (15 g, 77 mmol) and TBAF (92.7 mL, 1M in THF, 92.7 mL) in DMF (105 mL) was added BnBr (11.97 g, 0.07 mol). The reaction mixture was stirred at room temperature for 16 hours. After completion, the solvent was evaporated under vacuum and the residue was purified by silica gel column chromatography (DCM:MeOH=10:1) to give 4-2 (20.1 g, 70 mmol, 96% yield) as yellow oil.

To a solution of 4-2 (20 g, 70 mmol) in pyridine (60 mL) was added Ac₂O (30 mL). The reaction mixture was stirred at room temperature for 16 hours. After completion, the solvent was removed in vacuo and the residue was dissolved with DCM (400 mL), then washed with water (400 mL) and Brine (2×400 ml). The organic phase was dried over anhydrous Na₂SO₄, filtered, and evaporated under vacuum. The residue was purified by silica gel column chromatography to give desired product 4-3 (10 g, 31.6% yield) as a white solid.

To a solution of 4-3 (5.0 g, 11 mmol) in DCM (10 mL) was added HBr (11 mL, 48% in HBr, 44 mmol) dropwise. The reaction mixture was stirred at room temperature for 1.5 hours. The solvent was co-evaporated with toluene (3×500 mL) under vacuum. The residue was dissolved with DCM (500 mL) then washed with water (500 mL) and Brine (2×500 mL). The organic phase was dried over anhydrous Na₂SO₄, filtered, and evaporated under vacuum. The residue was purified by silica gel column chromatography to give desired product 4-4 (1.8 g, 3.8 mmol, 34.5% yield) as a white solid.

To a solution of 4-4 (5.0 g, 10.57 mol) and 4-5 (1.76 g, 10.57 mmol) in MeCN (50 mL) was added Ag₂O (2.45 g, 10.57 mmol). The reaction mixture was stirred room temperature for 2 hours under dark conditions. The solvent was removed under vacuum and the residue was purified by silica gel column chromatography to give 4-6 (4.2 g, 9.3 mmol, 88.1% yield) as a white solid.

To the mixture of 4-6 (5.1 g, 9.12 mmol) and silica gel (2.12 g) in CHCl₃ (50 mL) and iPrOH (10 mL) was added NaBH₄ (517 mg, 13.68 mmol). After stirred at 25′T for 1.5 h, the reaction mixture was filtered and the solvent was removed under vacuum. The residue was purified by silica gel column chromatography (PE:EA=1:1) to give 4-7 (4.8 g, 8.5 mmol, 93.8% yield) as a white solid.

Imidazole (5.2 g, 9.26 mmol) and DMAP (282 mg, 2.31 mmol) was added to a solution of 4-7 (5.2 g, 1.85 mmol) in DMF (30 mL) under N₂ atmosphere. After stirred for 5 min, the solution of TBSCl (2.08 g, 13.9 mmol) in DMF (30 mL) was added and the reaction solution was stirred at room temperature for 16 hours. The mixture was diluted with CH₂Cl₂, and the organic phase was washed with NH₄Cl(aq) (3×150 mL) and Brine (3×150 mL). The combined organic layers were dried over Na₂SO₄, filtered and the solvent removed in vacuo. The crude product was purified by the silica gel column chromatography (PE:EA=4:1) to give compound 4-8 (5 g, 79.9% yield) as a white solid.

To a solution of 4-8 (3.87 g, 5.7 mmol) in EtOH (20 mL) and H₂O (5 mL) was added Fe (1.59 g, 28.5 mmol) and NH₄Cl_{(aq)} (1.54 g, 28.5 mmol). After stirred at 65° C. for 2 hours, the reaction mixture was quenched with NH₄Cl(aq) (150 mL) and extracted with EA (2×150 mL). The combined organic layer was washed with Brine (2×150 mL), dried over anhydrous Na₂SO₄, filtered, and evaporated under vacuum. The residue was purified by the flash silica gel column chromatography to give 4-9 (3.0 g, 81.5% yield) as a yellow solid.

To a stirred solution of 4-9 (3.0 g, 4.65 mmol) and 4-10 (4.38 g, 3.75 mmol) in DCM (50 ml) was added EEDQ (5.7 g, 23.2 mmol). The mixture was stirred under N₂ atmosphere at room temperature 16 hours. The reaction was quenched with water (100 mL) and extracted with DCM (3×100 mL). The combined organic layer was washed with Brine (3×100 mL), dried over Na₂SO₄, filtered, and evaporated under vacuum. The residue was purified by silica gel column chromatography to afford 4-11 (3.2 g, 3.92 mmol, 84.3% yield) as a white solid.

To a solution of 4-11 in THF (20 mL) was added TEA3HF (5 mL). The reaction mixture was stirred at room temperature 1.5 hours. After completion, the solvent was removed and the residue was purified by silica gel column chromatography to give 4-12 (2 g, 66.4% yield) as a white solid.

To a solution of 4-12 (1.0 g, 1.42 mmol) and TEA (0.39 mL, 2.84 mol) in DCM (10 mL) was added 4-13 (2.16 g, 7.12 mmol). After stirred at 25° C. for 16 hours, the reaction was concentrated and the residue was purified by silica gel column chromatography to give 4-14 (930 mg, 1.07 mmol, 75.5% yield) as yellow oil.

To a solution of 4-14 (954 mg, 1.1 mmol) in DMF (10 mL) was added 4-15 (950 mg, 1.1 mmol), HOBT (148 mg, 1.1 mmol) and TEA (222 mg, 2.2 mmol). After stirred at 25° C. for 16 hours, the mixture was quenched with water, extracted with EtOAc (200 mL×3). The combined organic layer was washed with brine (100 mL×2), dried over Na₂SO₄, filtered and concentrated. The residue was purified by pre-HPLC to afford 4-16 (300 mg, 2 mmol, 18.0% yield).

To a solution of 4-16 (300 mg, 0.2 mmol) in MeOH (3 mL) was added 10% Pd/C (60 mg). The reaction mixture was stirred at 25° C. for 1 hour under 1 atm of H₂. The mixture was filtered and the solvent was evaporated to afford 4-17 (250 mg, 0.17 mmol, 88.0% yield).

To a solution of 4-17 (250 mg, 0.17 mmol) in MeOH (3 mL) was added Na₂CO₃ (60 mg, 0.6 mmol). The reaction mixture was stirred at 25° C. for 16 hours. After filtration, the solvent was removed to afford 4-18 (250 mg, 0.17 mmol, 99% yield).

To a solution of 4-18 (250 mg, 0.17 mmol) in DCM (5 mL) was added TFA (2 mL). After stirred at 25° C. for 1 hour, the solvent was removed and the residue was purified by pre-HPLC to get the desired product 4-19 (100 mg, 0.08 mmol, 44.4% yield).

To the water solution of compound 1 (150 mg, 6 mL, 25 mg/mL), the DMF solution of NHS (0.12 mg, 1.05 μmol), EDC (20 mg, 105.4 μmol) and 4-19 (23.4 mg, 42.2 μmol) were added slowly at 0° C., the reaction mixture was stirred at room temperature for 16 hours. After completion, the mixture was dialyzed with 5 KD MWCO to afford compound 4-20 as water solution (25 mL, 5 mg/mL).

To the water solution of compound 4-20 (25 mL, 5 mg/mL), 4N HCl (1.6 mL) was added slowly at 0° C., the reaction mixture was stirred at room temperature for 16 hours. After completion, the pH of the mixture was adjusted to 7 with saturated NaHCO₃ (aq) to afford the water solution of compound 4-21 which was used directly to the next step.

To the water solution of compound 4-21, NHS-PEG₄-Mal (32.6 mg, 5.25 μmol) in DMF (10 mL) was added slowly at 0° C., the reaction mixture was stirred at room temperature for 16 hours. After completion, the mixture was dialyzed with 5 KD MWCO to afford compound 4 as water solution (24 mL, 5 mg/mL).

### Example 5. Synthesis of Compound 5

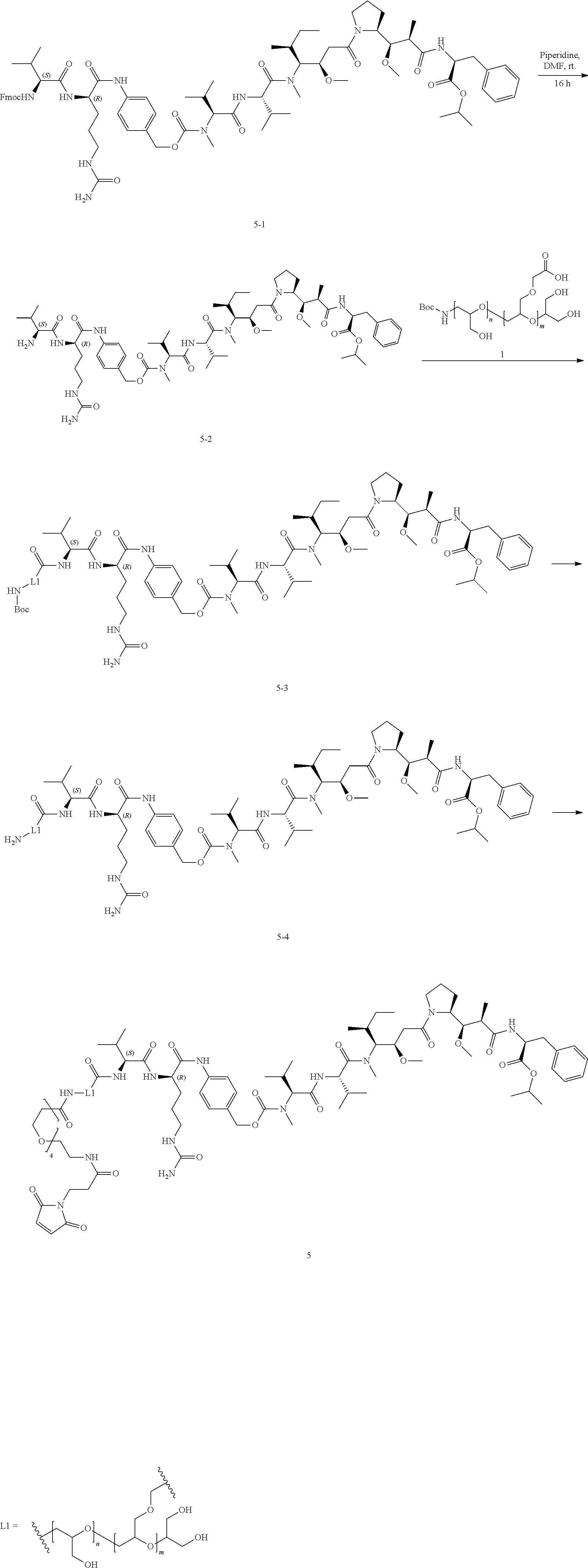

To the solution of 5-1 (500 mg, 0.36 mmol) in DMF (5 mL) was added piperidine (60.7 mg, 0.71 mmol) at 20 TC. Then the reaction was stirred at room temperature for 18 hours. After completion, the mixture was purified by prep-HPLC (TFA) to give the desired product 5-2 (200 mg, 0.169 mmol, 47.5% yield) as a white solid.

To the water solution of compound 1 (150 mg, 6 mL, 25 mg/mL), the DMF solution of NHS (0.12 mg, 1.05 μmol), EDC (20 mg, 105.4 μmol) and 5-2 (23.4 mg, 42.2 μmol) were added slowly at 0° C., the reaction mixture was stirred at room temperature for 16 hours. After completion, the mixture was dialyzed with 5 KD MWCO to afford compound 5-3 as water solution (25 mL, 5 mg/mL).

To the water solution of compound 5-3 (25 mL, 5 mg/mL), 4N HCl (1.6 mL) was added slowly at 0° C., the reaction mixture was stirred at room temperature for 16 hours. After completion, the pH of the mixture was adjusted to 7 with saturated NaHCO₃ (aq) to afford the water solution of compound 5-4 which was used directly to the next step.

To the water solution of compound 5-4, NHS-PEG₄-Mal (32.6 mg, 5.25 μmol) in DMF (10 mL) was added slowly at 0° C., the reaction mixture was stirred at room temperature for 16 hours. After completion, the mixture was dialyzed with 5 KD MWCO to afford compound 5 as water solution (16 mL, 5 mg/mL).

### Example 6. Synthesis of Compound 6

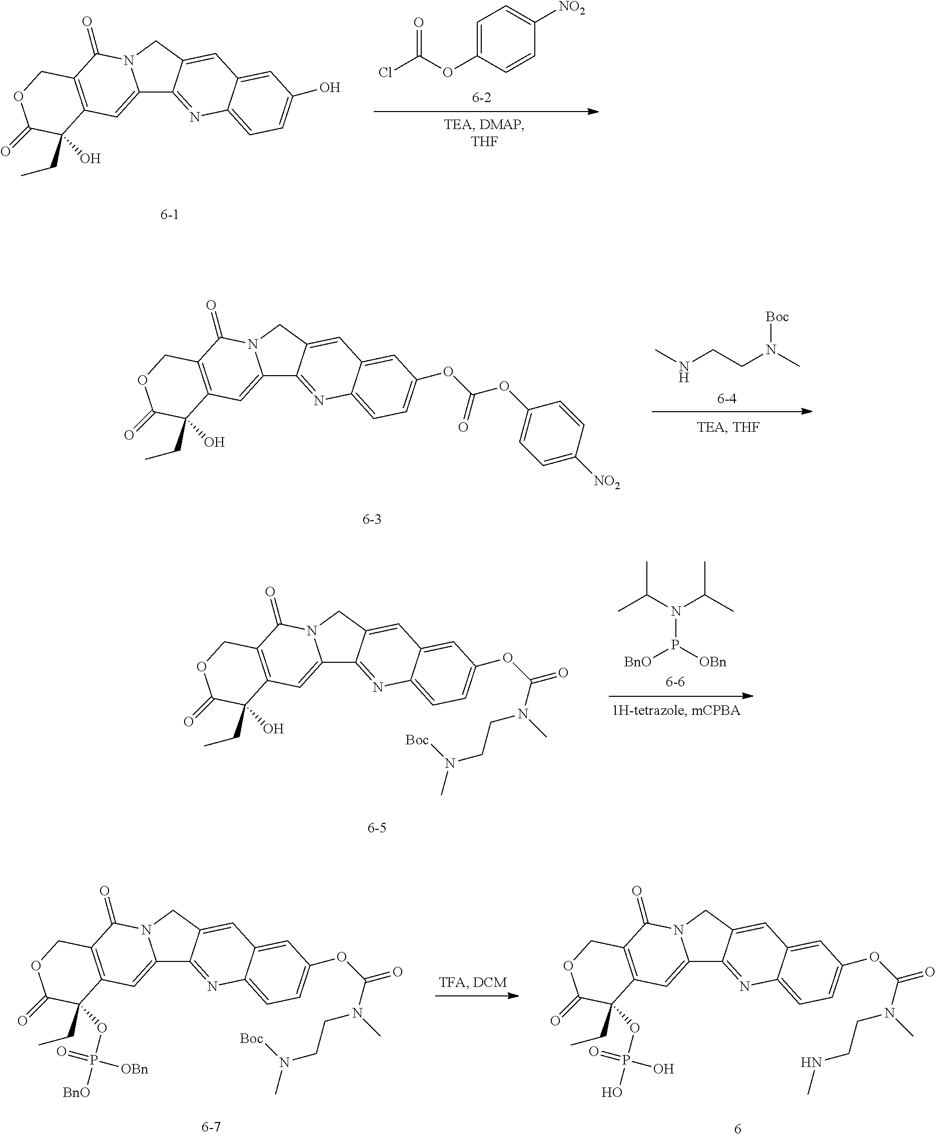

To a solution of 6-1 (2.5 g, 6.87 mmol) in THF (400 mL) was added 6-2 (5.55 g, 27.6 mmol) and TEA (10 mL) under N₂. The reaction was stirred at room temperature for 16 hours. The reaction was diluted with water (100 mL) and extracted with EtOAc (50 mL×3). The combined organic phases were washed with brine (100 mL), dried over Na₂SO₄ and concentrated under vacuum to give the residue, which was purified by silica gel column chromatography to give the desired product 6-3 (2.5 g, 4.7 mmol, 69% yield) as an off white solid.

To a solution of 6-3 (3.3 g, 6.24 mmol) in TH (300 mL) was added 6-4 (1.5 g, 7.9 mmol) and TEA (815 mg, 8.05 mmol) under N₂. The reaction was stirred at room temperature for 16 hours. The reaction was quenched with water (100 mL) and extracted with EtOAc (100 mL×3). The combined organic phases were washed with brine (100 mL), dried over Na₂SO₄ and concentrated under vacuum to give the residue, which was purified by silica gel column chromatography to give the desired product 6-5 (2.1 g, 3.6 mmol, 58% yield) as a white solid.

To a solution of 6-5 (1.0 g, 1.73 mmol) in DCM (50 mL) was added tetrazole (242 mg, 3.46 mmol) at room temperature under N₂. After stirred for 0.5 hour, 6-6 (1.07 g, 3.11 mmol) was added dropwise at 0T. The resulting solution was stirred at room temperature for 16 hours. After completion, mCPBA (559 mg, 2.59 mmol) was added at 09T and stirred for another 16 h. The reaction was then diluted with water (100 mL) and the organic layer was washed with aqueous sodium metabisulfite, sodium bicarbonate solution and dried over Na₂SO₄. The mixture was filtered and solvent was evaporated, the residue was purified by prep-HPLC (water/ACN, with 0.1% TFA) to obtain the desired product 6-7 (1.0 g, 69% yield) as a yellow solid.

To a solution of 6-7 (1.0 g, 1.19 mmol) in DCM (20 mL) was added TFA (20 mL) at 0° C. under N₂. The reaction was stirred for 16 hours at room temperature. The reaction was concentrated under vacuum to give the crude, which was purified by prep-HPLC (TFA) to give the desired product 6 as a yellow solid.

¹H NMR (400 MHz, CDCl3) δ 8.21 (s, I H), 7.93 (dd, J=25.8, 9.1 Hz, 2H), 7.59 (s, 1H), 7.43 (s, 1H), 5.60 (d, J=16.5 Hz, 1H), 5.40 (d, J=16.6 Hz, 1H), 5.18 (s, 2H), 3.94 (s, 1H), 3.40 (s, 1H), 3.29-3.17 (m, 2H), 3.08 (d, J=15.4 Hz, 3H), 2.78 (s, 3H), 2.14 (d, J=7.3 Hz, 2H), 1.03 (t, J=7.2 Hz, 3H).

### Example 7. Synthesis of Compound 7

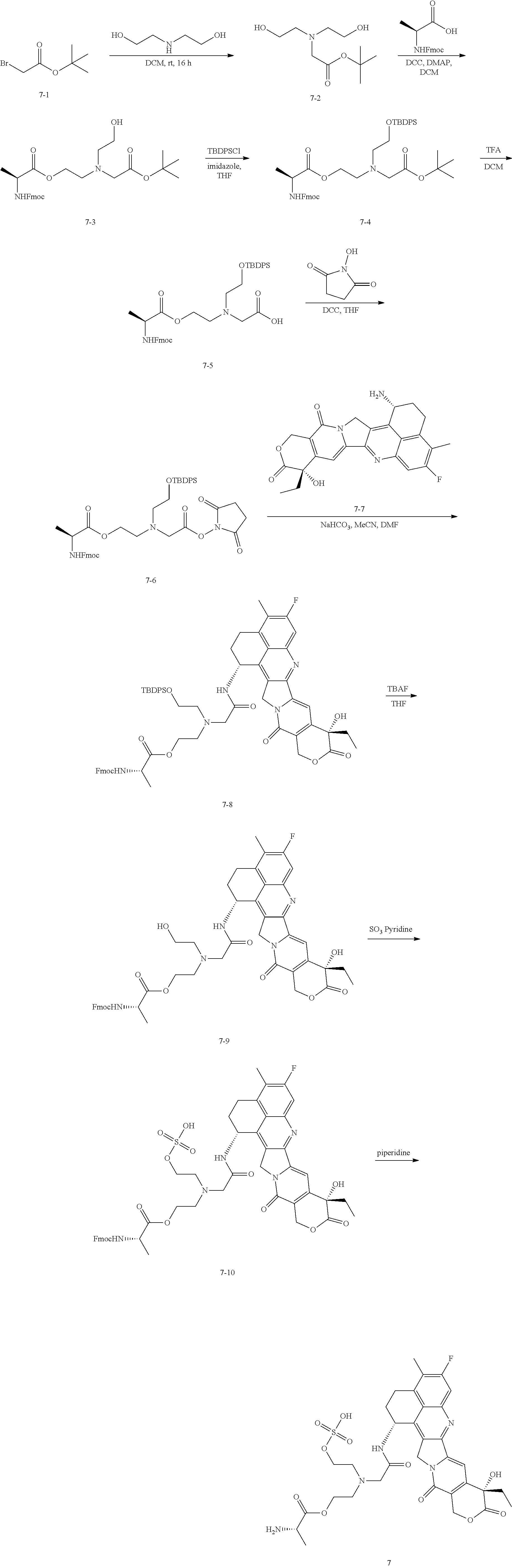

To the solution of 7-1 (25.0 g, 114.01 mmol) in DCM (250 mL) was added 2,2′-azanediylbis(ethan-1-ol) (39.04 g, 125.41 mmol), DMAP (1.39 g, 11.4 mmol) and DCC (3.56 g, 136.81 mmol). After stirred at room temperature for 16 hours, the reaction mixture was diluted with water (400 mL) and extracted with EA (700 mL), the organic phase was dried with Na₂SO₄, filtered, and concentrated. The residue was purified by silica gel column chromatography to afford the desired product 7-2 as yellow oil (25 g, 114 mmol, 89%).

To a solution of 7-2 (25.0 g, 114.01 mmol) in DCM (250 mL) was added (((9H-fluoren-9-yl)methoxy)carbonyl)-L-alanine (39.04 g, 125.41 mmol), DMAP (1.39 g, 11.4 mmol) and DCC (3.56 g, 136.81 mmol). After stirred at room temperature for 16 hours, the reaction mixture was diluted with water (400 mL), and extracted with EA (700 mL). The organic phase was dried with Na₂SO₄, filtered and concentrated. The residue was purified by silica gel column chromatography to get the desired product 7-3 as colorless oil (25.5 g, 49.7 mmol, 43%).

To a solution of 7-3 (23 g, 44.87 mmol) in THF (200 mL) was added TBDPSCl (13.57 g, 49.36 mmol) and imidazole (6.11 g, 89.74 mmol). After stirred at room temperature for 12 hours, the reaction mixture was concentrated to afford the crude product 7-4 as colorless oil (39 g, 51.9 mmol, 100%) which was used for next step without further purification.

To a solution of 7-4 (39 g, 51.93 mmol) in DCM (150 mL) was added TFA (150 mL) and stirred at room temperature 12 hours. The solvent was removed under vacuum and the residue was purified by flash column chromatography with DCM/MeOH (20/1, v/v) to afford the desired product 7-5 as a white solid (21 g, 30.2 mmol, 58%).

To a solution of 7-5 (5 g, 7.2 mmol) in DCM (50 mL) was added 1-hydroxypyrrolidine-2,5-dione (1.24 g, 10.79 mmol) and DCC (5.94 g, 28.8 mmol) at room temperature and stirred for 16 hours. The solvent was removed under vacuum and the residue was purified by flash column chromatography with PE/EtOAc (1/1, v/v) to afford the desired product 7-6 as colorless oil (5 g, 6.3 mmol, 88%).

To a solution of 7-6 in DMF (20 mL) was added 7-7 (1.2 g, 1.52 mmol), and DIEA (653 mg). The reaction mixture was stirred at room temperature for 16 hours. The mixture was diluted with water and extracted with EA. The organic phase was dried with Na₂SO₄, filtered and evaporated under reduced pressure. The residue was purified by flash silica gel column chromatography with DCM/MeOH (20/1, v/v) to afford the desired product 7-8 as a green solid. (750 mg, 0.67 mmol, 67%).

To a solution of 7-8 (750 mg, 0.67 mmol) in THF (8 mL) was added TBAF (2 mL, 1M in THF, 2 mmol) dropwise at room temperature. The reaction mixture was stirred at room temperature for 16 hours. The mixture was concentrated under vacuum and the residue was purified by flash silica gel column chromatography with DCM/MeOH (20/1, v/v) to afford the desired product 7-9 (500 mg, 0.57 mmol, 85% yield) as black solid.

To a solution of 7-9 (500 mg, 0.57 mmol) in pyridine (5 mL) was added SO₃Pyridine (455 mg, 2.86 mmol) at room temperature and the mixture was stirred for 16 hours. Then the solvent was removed under vacuum and the residue was purified by silica gel column chromatography with MeOH/H₂O (1/1, v/v) to afford 7-10 as a yellow solid. (500 mg, 0.52 mmol, 92%).

To a solution of 7-10 (500 mg, 0.52 mmol) in DMF (3 mL) was added piperidine (89.26 mg, 1.05 mmol) at room temperature and the mixture was stirred for 16 hours. The solvent was removed under vacuum and the residue was purified by C18 column chromatography (MeOH/H₂O (1/1, v/v)) to afford the desired product 7 as a yellow solid (229 mg, 0.31 mmol, 60%).

1H NMR (400 MHz, DMSO) δ 8.92 (s, 1H), 8.31 (s, 3H), 7.78 (d, J=10.9 Hz, 1H), 7.30 (s, 1H), 5.67-5.12 (m, 5H), 4.44 (s, 2H), 3.96 (d, J=62.4 Hz, 6H), 3.59-2.98 (m, 6H), 2.44-2.04 (m, 5H), 1.85 (tt, J=14.0, 7.1 Hz, 2H), 1.46-1.17 (m, 3H), 0.86 (t, J=7.3 Hz, 3H).

### Example 8. Synthesis of Compound 8

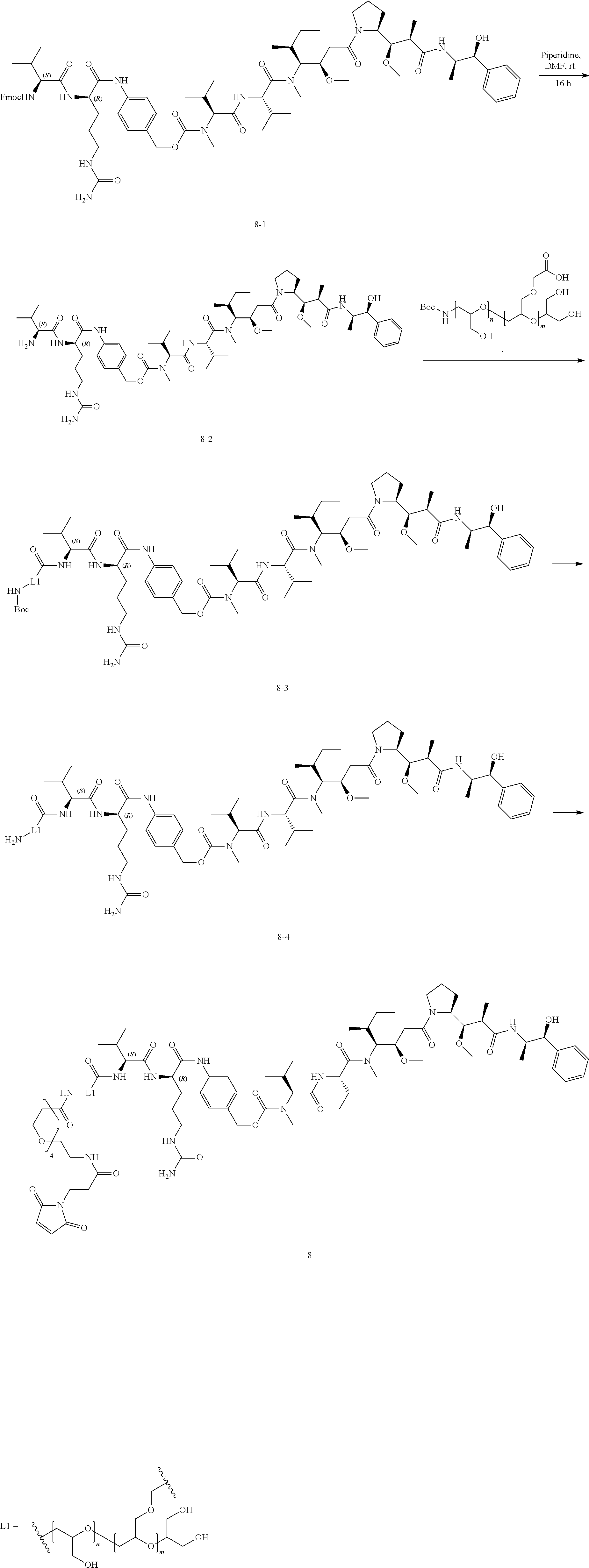

To the solution of 8-1 (500 mg, 0.36 mmol) in DMF (5 mL) was added piperidine (60.7 mg, 0.71 mmol) at 20 Tc. Then the reaction was stirred at room temperature for 18 hours. After completion, the mixture was purified by prep-HPLC (TFA) to give the desired product 8-2 (200 mg, 0.169 mmol, 47.5% yield) as a white solid.

To the water solution of compound 1 (150 mg, 6 mL, 25 mg/mL), the DMF solution of NHS (0.12 mg, 1.05 μmol), EDC (20 mg, 105.4 μmol) and 8-2 (23.4 mg, 42.2 μmol) were added slowly at 0° C., the reaction mixture was stirred at room temperature for 16 hours. After completion, the mixture was dialyzed with 5 KD MWCO to afford compound 8-3 as water solution (25 mL, 5 mg/mL).

To the water solution of compound 8-3 (25 mL, 5 mg/mL), 4N HCl (1.6 mL) was added slowly at 0° C., the reaction mixture was stirred at room temperature for 16 hours. After completion, the pH of the mixture was adjusted to 7 with saturated NaHCO₃ (aq) to afford the water solution of compound 8-4 which was used directly to the next step.

To the water solution of compound 8-4, NHS-PEG₄-Mal (32.6 mg, 5.25 μmol) in DMF (10 mL) was added slowly at 0° C., the reaction mixture was stirred at room temperature for 16 hours. After completion, the mixture was dialyzed with 5 KD MWCO to afford compound 8 as water solution (16 mL, 5 mg/mL).

### Example 9. Synthesis of ADC-1

To a Trastuzumab (5 mg, 2.5 mg/ml) buffer solution (PB, histidine, TEAA, sodium acetate buffer), TCEP (8 μL, 17.4 mM, 2 eq) was added. After reacted for 1.5 hours at room temperature, 6 eq. of Compound 2 was added and reacted for another 1.5 hours. Then 50 eq. of L-cysteine was added to quench the reaction. The mixture was purified via cation exchange column to afford ADC-1 (3 mg, 2 mg/ml). DAR was determined by UV spectrometer.

### Example 10. Synthesis of ADC-2

To a Trastuzumab (5 mg, 2.5 mg/ml) buffer solution (PB, histidine, TEAA, sodium acetate buffer), TCEP (8 μL, 17.4 mM, 2 eq) was added. After reacted for 1.5 hours at room temperature, 6 eq. of Compound 3 was added and reacted for another 1.5 hours. Then 50 eq. of L-cysteine was added to quench the reaction. The mixture was purified via cation exchange column to afford ADC-2 (3 mg, 2 mg/ml). DAR was determined by UV spectrometer.

### Example 11. Synthesis of ADC-3

To a Trastuzumab (5 mg, 2.5 mg/ml) buffer solution (PB, histidine, TEAA, sodium acetate buffer), TCEP (8 μL, 17.4 mM, 2 eq) was added. After reacted for 1.5 hours at room temperature, 6 eq. of Compound 4 was added and reacted for another 1.5 hours. Then 50 eq. of L-cysteine was added to quench the reaction. The mixture was purified via cation exchange column to afford ADC-3 (3 mg, 2 mg/ml). DAR was determined by UV spectrometer.

### Example 12. Synthesis of ADC-4

To a Trop2 antibody Trop2-1 (5 mg, 2.5 mg/ml) buffer solution (PB, histidine, TEAA, sodium acetate buffer), TCEP (8 μL, 17.4 mM, 2 eq) was added. After reacted for 1.5 hours at room temperature, 6 eq. of Compound 2 was added and reacted for another 1.5 hours. Then 50 eq. of L-cysteine was added to quench the reaction. The mixture was purified via cation exchange column to afford ADC-4 (3 mg, 2 mg/ml). DAR was determined by UV spectrometer.

### Example 13. Synthesis of ADC-5

To a Trop2 antibody Trop2-1 (5 mg, 2.5 mg/ml) buffer solution (PB, histidine, TEAA, sodium acetate buffer), TCEP (8 μL, 17.4 mM, 2 eq) was added. After reacted for 1.5 hours at room temperature, 6 eq. of Compound 3 was added and reacted for another 1.5 hours. Then 50 eq. of L-cysteine was added to quench the reaction. The mixture was purified via cation exchange column to afford ADC-5 (3 mg, 2 mg/ml). DAR was determined by UV spectrometer.

### Example 14. Synthesis of ADC-6

To the Trop2 antibody Trop2-1 (5 mg, 2.5 mg/ml) buffer solution (PB, histidine, TEAA, sodium acetate buffer), TCEP (8 μL, 17.4 mM, 2 eq) was added. After reacted for 1.5 hours at room temperature, 6 eq. of Compound 4 was added and reacted for another 1.5 hours. Then 50 eq. of L-cysteine was added to quench the reaction. The mixture was purified via cation exchange column to afford ADC-6 (3 mg, 2 mg/ml). DAR was determined by UV spectrometer.

### Example 15. Synthesis of ADC-7

To the Trop2 antibody Trop2-2 (5 mg, 2.5 mg/ml) buffer solution (PB, histidine, TEAA, sodium acetate buffer), TCEP (8 μL, 17.4 mM, 2 eq) was added. After reacted for 1.5 hours at room temperature, 6 eq. of Compound 2 was added and reacted for another 1.5 hours. Then 50 eq. of L-cysteine was added to quench the reaction. The mixture was purified via cation exchange column to afford ADC-7 (3 mg, 2 mg/ml). DAR was determined by UV spectrometer.

### Example 16. Synthesis of ADC-8

To the Trop2 antibody Trop2-2 (5 mg, 2.5 mg/ml) buffer solution (PB, histidine, TEAA, sodium acetate buffer), TCEP (8 μL, 17.4 mM, 2 eq) was added. After reacted for 1.5 hours at room temperature, 6 eq. of Compound 3 was added and reacted for another 1.5 hours. Then 50 eq. of L-cysteine was added to quench the reaction. The mixture was purified via cation exchange column to afford ADC-8 (3 mg, 2 mg/ml). DAR was determined by UV spectrometer.

### Example 17. Synthesis of ADC-9

To the Trop2 antibody Trop2-2 (5 mg, 2.5 mg/ml) buffer solution (PB, histidine, TEAA, sodium acetate buffer), TCEP (8 μL, 17.4 mM, 2 eq) was added. After reacted for 1.5 hours at room temperature, 6 eq. of Compound 4 was added and reacted for another 1.5 hours. Then 50 eq. of L-cysteine was added to quench the reaction. The mixture was purified via cation exchange column to afford ADC-9 (3 mg, 2 mg/ml). DAR was determined by UV spectrometer.

### Example 18. Cell Based Binding of Anti-HER2 Antibody and Anti-HER2 ADCs to HER2 Expressing Tumor Cell Lines

The HER2 positive cell lines SKBR-3 cells (Breast cancer, ATCC No. HTB30) and NCI-N87 cells (Gastric cancer, ATCC No. CRL-5822) were used for the in-vitro cell binding evaluation of exemplary anti-HER2 ADCs ADC-2 (two batches, namely ADC-2-1 and ADC-2-2) and ADC-3. The marketed anti-HER2 ADC drugs, DS-8201a from Daiichi Sankyo and T-DM1 from Genentech (Roche), were used as the positive control ADCs, and native antibody Trastuzumab (Genentech (Roche)) was used for the free antibody control.

Briefly, SKBR-3 cells and NCI-N87 cells were respectively maintained in McCoy's 5a medium (Cat. No. 30-2007, ATCC) and RPMI-1640 medium (Cat. No. abs9468, Absin) supplemented with 10% fetal bovine serum (FBS) (Cat. No. FSP500, Excell Bio), 2 mM L-glutamine (Cat. No. 25030081, ThermoFisher) and 1% Penicillin-Streptomycin Solution (BL505A, Biosharp Life Sciences). Cells were cultured at 37° C. in an atmosphere of 5% CO₂ in air, and harvested from flask for the assay when the confluence reached 70%-80%, 100 μL of 2×10⁶ cells/mL of cells per well in 96-well cell culture plate were incubated with Trastuzumab and test anti-HER2 ADCs in a 5-fold serial dilutions starting from 100 nM to 0.00128 nM for one hour on ice. After being washed twice with 200 μL per well of FACS buffer (PBS with 1% FBS), cells were incubated with 100 μL 1× secondary antibody PE anti-human IgG Fc Antibody (Cat. No. 366904, Biolegend) for 30-60 mins on ice. Cells were washed twice with 200 μL of FACS buffer and re-suspended with 200 μL FACS buffer for further detection by CytoFlex Cytometry (Beckman Coulter). Data were analyzed with the built-in analysis software of CytoFlex.

The cell binding results were shown in FIG. 1. The binding capacities of the tested anti-HER2 ADCs to SKBR-3 cells and NCI-N87 cells were efficient and compatible to Trastuzumab.

### Example 19. Antibody Mediated Internalization Effect in HER2 Expressing Tumor Cells Using FACS Analysis

The HER2 positive cell lines SKBR-3 cells and NCI-N87 cells and the test anti-HER2 ADCs (exemplary anti-HER2 ADCs ADC-2-1, ADC-2-2 and ADC-3, and the positive control ADCs DS-8201a and T-DM1) as used in Example 18 were also used in the following internalization assay. The culture conditions of two cell lines are the same as in Example 18.

Cells were harvested with 0.25% Trysin/EDTA (Tl 320, Solarbio) and adjusted the cell concentration to 4×10⁶ cells/mL with 1×PBS containing 1% FBS buffer. 100 μL cells per well were then plated in the 96-well cell culture plates and incubated with Trastuzumab and the test anti-HER2 ADCs at a final concentration 100 nM for seven time points (0, 0.5, 2, 4, 8, 16, and 24 hours). The mixtures were incubated at 4° C. in an atmosphere of 5% CO₂ in air. At specific time points, cells were immediately washed twice and suspended in 100 μL cold wash buffer containing 2% paraformaldehyde for 30 mins. Cells were washed twice and then incubated with 1×PE anti-human IgG Fc Antibody (Cat. No. 366904, Biolegend) at 4° C. and 5% CO₂ for another one hour. After the two-step washing by washing buffer, cells were re-suspended in 100 μL FACS buffer, CytoFlex Cytometry (Beckman Coulter) was used to detect the fluorescent signals of cells. The internalization ratio at several time points were calculated as the missing rate of median fluorescence intensity (MFI) values at specific time point in that at 0 hours. Curves of internalization rate versus incubation time (hours) were plotted with the Graphpad Prism Software.

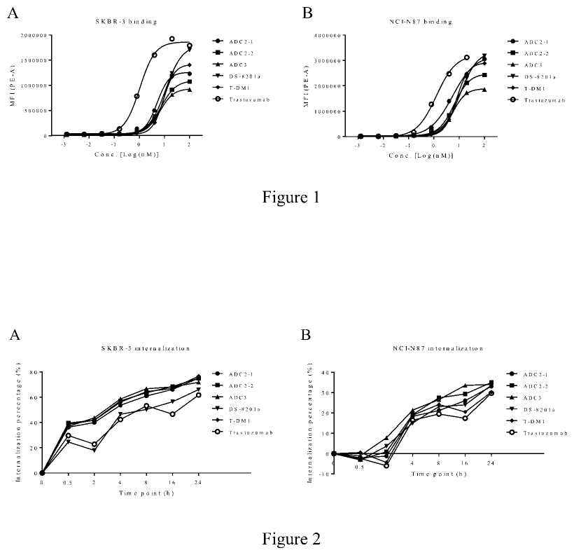

The results for SKBR-3 cells and NCI-N87 cells were shown in Tables 1 and 2 as well as FIG. 2. All the test anti-HER2 ADCs could effectively mediate the internalization into HER2 positive cells (SKBR-3 cells and NCI-N87 cells), with the internalization rate of >60% (SKBR-3 cells) and >30% (NCI-N87 cells) within 24 hours. The internalization results indicated that the conjugation could not change the target mediated internalization.

TABLE 1
MFI value of internalization assay on SKBR-3 cells

| | ADC-2- | ADC-2- | | DS- | | |
|---|---|---|---|---|---|---|
| MFI | 1 | 2 | ADC-3 | 8201a | T-DM1 | Trastuzumab |
| 0 h | 81296.3 | 70886.1 | 58383.9 | 111493.1 | 89030.9 | 114620.8 |
| 0.5 h | 51815.9 | 42850.6 | 36842.6 | 84105.2 | 54683.9 | 80551.7 |
| 2 h | 48758.2 | 41203.7 | 32890.5 | 91504.5 | 51777.7 | 88422.6 |
| 4 h | 37648.4 | 30356.6 | 24160.4 | 59683.6 | 39019.6 | 66067.7 |
| 8 h | 31700.9 | 25784.3 | 19417.6 | 55324.7 | 31583.1 | 53596.8 |
| 16 h | 27502.4 | 22283.5 | 18533.7 | 48587.4 | 29393.7 | 61201.3 |
| 24 h | 20177 | 17657.4 | 16436.4 | 37770 | 20671.3 | 43793.5 |

TABLE 2
MFI value of internalization assay on NCI-N87 cells

| MFI | ADC-2-1 | ADC-2-2 | ADC-3 | DS-8201a | T-DM1 | Trastuzumab |
|---|---|---|---|---|---|---|
| 0 h | 382098.4 | 322860.4 | 302930 | 470434.6 | 455385 | 494263.3 |
| 0.5 h | 389864.7 | 332284.1 | 306071.1 | 485012.7 | 452742.7 | 505817.1 |
| 2 h | 386741.6 | 319953.5 | 279387.2 | 453325.2 | 475100.1 | 523528.4 |
| 4 h | 311931.9 | 260945 | 238134.4 | 399661.6 | 369328.5 | 412925.1 |
| 8 h | 301151.8 | 234073.9 | 222454.3 | 361668.5 | 345220.2 | 398055.9 |
| 16 h | 282719.9 | 228232.9 | 201278 | 357611.1 | 362105.3 | 408260.5 |
| 24 h | 255656.2 | 209783 | 199868.8 | 312219.9 | 317532.7 | 347311 |

### Example 20. In Vitro Cytotoxic Assays of Anti-HER2 ADCs on HER2-Expressing Cells

Cytotoxicity of exemplary anti-HER2 ADCs was investigated using SKBR-3 cells (Breast cancer, ATCC No. HTB30) and NCI-N87 cells (Gastric cancer, ATCC No. CRL-5822) and compared to the positive control ADCs DS-8201a and T-DM1.

In brief, 5,000 cells/well of SKBR-3 cells and NCI-N87 cells were platted in white clear 96-well assay plates (Cat. No. 6005182, PerkinElmer) (excluding edge wells, which contained medium only) in 90 μL basic culture medium (McCoy's 5a medium for SKBR-3 cells and RPMI-1640 medium for NCI-N87 cells) containing 10% FBS, 1% Penicillin-Streptomycin Solution (BL505A, Biosharp Life Sciences), and 2 mM L-Glutamine (Cat. No. 25030081, ThermoFisher) and were grown at 37° C. in a humidified incubator at 5% CO₂ atmosphere. After incubation overnight, each test anti-HER2 ADC or Trastuzumab was added to the respective wells in an amount of 10 μL of 10-concentrate tests from 100 nM to 0.015 nM. After additional 72-hour incubation, plates were removed from incubator and equilibrated to room temperature. After approximately 30 mins, 50 μL of CellTiter-Glo® 2.0 Luminescent Cell Viability Reagent (Cat. No. G7573, Promega) were added to each well. After shaking the plates at 450 rpm for 3 mins followed by 10-min incubation without shaking, luminescence was measure on the Envision plate reader (Equipment No. 2104, PerkinElmer) with integration time of 250 ms per well. Curves of luminescence versus ADC concentration (μM) were fitted with GraphPad Prism Software.

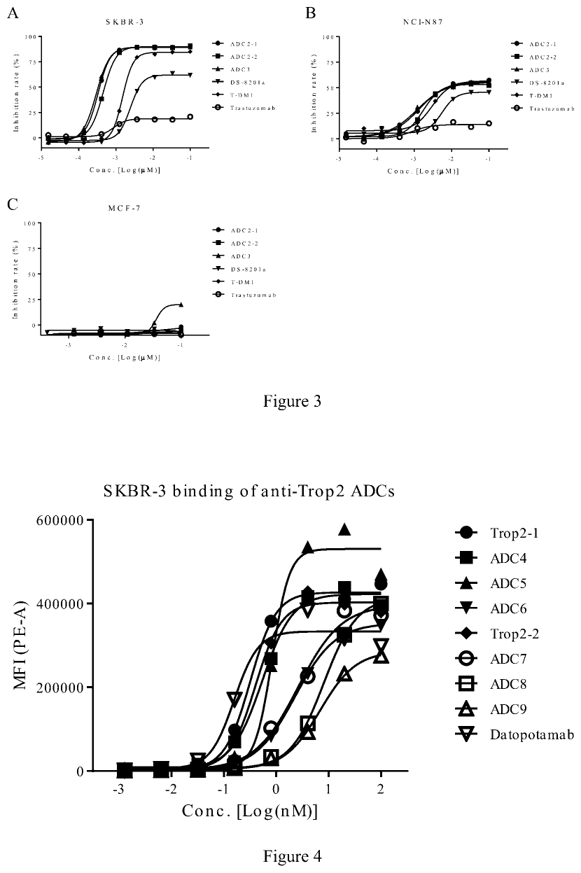

The results of the in vitro cytotoxic assays are shown in FIG. 3. The exemplary anti-HER2 ADCs (ADC-2-1, ADC-2-2, and ADC-3) showed a better cytotoxic activity than the positive control ADCs DS-8201a and T-DM1 on both HER positive NCI-N87 cells (FIG. 3A) and SKBR-3 cells (FIG. 3B), while the cytotoxic activity was only observed at very high concentrations of ADCs (especially that of ADC-3) in HER2 negative MCF-7 cells (FIG. 3C). The control native antibody Trastuzumab had only limited cytotoxic activity even at high concentrations.

### Example 21. Cell Based Binding of Anti-Trop2 Antibodies and Anti-Trop2 ADCs to Trop2 Expressing Tumor Cells

The Trop2 positive SKBR-3 cells (Breast cancer, ATCC No. HTB30) were used for the in-vitro cell binding evaluation of exemplary anti-Trop2 ADCs ADC-4, ADC-5, ADC-6, ADC-7, ADC-8 and ADC-9. The native human IgG1 antibody Datopotama of DS-1062 from Daiichi Sankyo was used for the free antibody control. The protocol of binding assay was same as that in Example 18.

Briefly, SKBR-3 cells were maintained in McCoy's 5a medium (Cat. No. 30-2007, ATCC) supplemented with 10% FBS (Cat. No. FSP500, Excell Bio), 2 mM L-glutamine (Cat. No. 25030081, ThermoFisher) and 1% Penicillin-Streptomycin Solution (BL505A, Biosharp Life Sciences). Cells were cultured at 37° C. in an atmosphere of 5% CO₂ in air, and harvested from flask for the assay when the confluence reached 70%-80%, 100 μL of 2×10⁶ cells/mL of cells per well in 96-well cell culture plate were incubated with Trop2 antibodies Trop2-1, Trop2-2, and Datopotamab, as well as the exemplary anti-Trop2 ADCs in a 5-fold serial dilutions starting from 100 nM to 0.00128 nM for one hour on ice. After being washed twice with 200 μL per well of FACS buffer, cells were incubated with 100 μL 1× secondary antibody PE anti-human IgG Fc Antibody (Cat. No. 366904, Biolegend) for 30-60 mins on ice. Cells were washed twice with 200 μL of FACS buffer and re-suspended with 200 μL FACS buffer for further detection by CytoFlex Cytometry (Beckman Coulter). Data were analyzed with the built-in analysis software of CytoFlex.

The cell binding results were shown in FIG. 4. The exemplary anti-Trop2 ADCs could effectively bind to SKBR-3 cells with comparable binding capacity as their native Trop2 antibody Trop2-1 and Trop2-2, which indicated that the conjugation has sparely binding influences on the target binding activities.

### Example 22. Antibody Mediated Internalization Effect in Trop2 Expressing Tumor Cells Using FACS Analysis

The Trop2 positive cell line SKBR-3 cells (Breast cancer, ATCC No. HTB30) were used for the following internalization assay. The culture conditions are the same as in Example 21.

Cells were harvested with 0.25% Trysin/EDTA (T1320, Solarbio) and adjusted the cell concentration to 4×10⁶ cells/mL with 1×PBS containing 1% FBS buffer. 100 μL cells per well were then plated in the 96-well cell culture plates and incubated with Trop2 antibodies Trop2-1, Trop2-2, Datopotamab, and Sacituzumab, as well as exemplary anti-Trop2 ADCs (ADC-4, ADC-5, ADC-6, ADC-7, ADC-8 and ADC-9) at final concentration 100 nM for seven time points (0, 0.5, 2, 4, 8, 16, and 24 hours). The mixtures were incubated at 4° C. in an atmosphere of 5% CO₂ in air. At specific time points, cells were immediately washed twice and suspended in 100 μL cold wash buffer containing 2% paraformaldehyde for 30 mins. Cells were washed twice and then incubated with 1×PE anti-human IgG Fc Antibody (Cat. No. 366904, Biolegend) at 4° C. and 5% CO₂ for another one hour. After the two-step washing by washing buffer, cells were re-suspended in 100 μL FACS buffer, CytoFlex Cytometry (Beckman Coulter) was used to detect the fluorescent signals of cells. The internalization ratio at several time points were calculated as the missing rate of MFI values at specific time point in that at 0 hours. Curves of internalization rate versus incubation time (hours) were plotted with the Graphpad Prism Software.

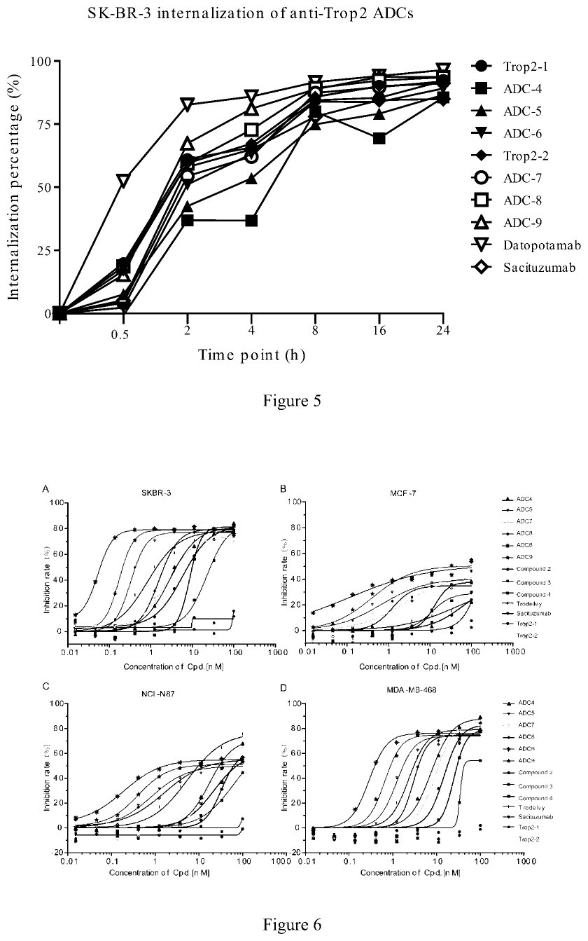

The results were shown in Table 3 and FIG. 5. The exemplary anti-Trop2 ADCs could effectively mediate the internalization into SKBR-3 cells, with the internalization rate of >60% within 8 hours. The internalization results indicated that the conjugation could not change the target mediated internalization.

TABLE 3
MFI value of internalization assay on SKBR-3 cells

| MFI | Trop2-1 | ADC-4 | ADC-5 | ADC-6 | Trop2-2 | ADC-7 | ADC-8 | ADC-9 | Datopotamab | Sacituzumab |
|---|---|---|---|---|---|---|---|---|---|---|
| 0 h | 350877.3 | 253057.4 | 197184.5 | 214459.9 | 202392.5 | 155471.5 | 90165.1 | 105345.6 | 237324.4 | 216386 |
| 0.5 h | 280839.1 | 255084.3 | 182352.4 | 209643.3 | 167809.1 | 148921.1 | 73393.6 | 89224.6 | 112896.1 | 205198.9 |
| 2 h | 136432.5 | 159723.1 | 113396.9 | 104988.9 | 81928.8 | 70940.6 | 36635.6 | 34421.6 | 41192.3 | 90975.5 |
| 4 h | 120178 | 160017.5 | 91722.5 | 78943.3 | 66187.5 | 58877.4 | 24539 | 19899.7 | 33418.6 | 75749.9 |
| 8 h | 54526.5 | 50624.5 | 49440.6 | 34608.2 | 28769.9 | 19651.6 | 9695.9 | 11816.6 | 19928.3 | 47695.7 |
| 16 h | 51083.2 | 77416.1 | 41163.3 | 34844.2 | 20174.8 | 16069.8 | 6937.3 | 6680.6 | 14134.9 | 33528.1 |
| 24 h | 27612.4 | 36599.2 | 26440.6 | 23360.7 | 17971.6 | 12512 | 5802.5 | 6618.8 | 8497.3 | 32475 |

### Example 23: In Vitro Cytotoxic Assays of Anti-Trop2 ADCs on Trop2-Expressing Cancer Cells

Cytotoxicity of exemplary free linker-payloads (Compounds 2, 3 and 4), free Trop2 antibodies (Trop2-1, Trop2-2, Datopotamab, and Sacituzumab), exemplary anti-Trop2 ADCs (ADC-4, ADC-5, ADC-6, ADC-7, ADC-8 and ADC-9) and Trodelvy (an anti-Trop2 ADC approved by FDA) as positive control was evaluated on SKBR-3 cells (Breast cancer, ATCC No. HTB30), NCI-N87 cells (Gastric cancer, ATCC No. CRL-5822), MCF-7 cells (Breast cancer, ATCC No. HTB-22), and MDA-MB-468 cells (Breast cancer, ATCC No. HTB-132).

In brief, 5,000 cells/well were platted in white clear 96-well assay plates (Cat. No. 6005182, PerkinElmer) (excluding edge wells, which contained medium only) in 90 μL basic culture medium (McCoy's 5a medium for SKBR-3 cells, RPMI-1640 medium for NCI-N87 cells, EMEM medium for MCF-7 cells, and Leibovitz's L-15 medium for MDA-MB-468 cells) containing 10% FBS, I % Penicillin-Streptomycin Solution (BL505A, Biosharp Life Sciences), and 2 mM L-Glutamine (Cat. No. 25030081, ThermoFisher) and were grown at 37° C. in a humidified incubator at 5% CO₂ atmosphere. After incubation overnight, the exemplary anti-Trop2 ADCs, Trodelvy, the free Trop2 antibodies, and the free linker-payloads were added to the respective wells in an amount of 10 μL of 10×concentrate tests from 100 nM to 0.015 nM. After additional 72-hour incubation, plates were removed from incubator and equilibrated to room temperature. After approximately 30 mins, 50 μL of CellTiter-Glo® 2.0 Luminescent Cell Viability Reagent (Cat. No. G7573, Promega) were added to each well. After shaking the plates at 450 rpm for 3 mins followed by 10-min incubation without shaking, luminescence was measure on the Envision plate reader (Equipment No. 2104, PerkinElmer) with integration time of 250 ms per well. Curves of luminescence versus ADC concentration (μM) were fitted with GraphPad Prism Software.

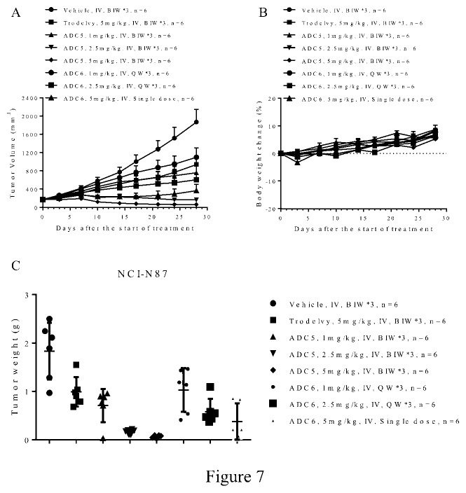

The results of the in vitro cytotoxic assays are shown in FIG. 6 and Table 4. All exemplary anti-Trop2 ADCs showed cytotoxic activity on the test cell lines. ADC-6, ADC-9, and ADC-5 showed a better cytotoxic activity than Trodelvy on the test cell lines, and ADC-4 presented a comparable target cytotoxicity as Trodelvy on the test cell lines. The free linker-payloads Compounds 2-4 had only limited cytotoxic activity even at high concentrations. In addition, the native antibodies Sacituzumab, Trop2-1, and Trop2-2 presented no target cell killing effect on these four Trop-2 expressing cells.

TABLE 4
The in-vitro cytotoxicity of several anti-Trop2 ADCs on SKBR-3, NCI-N87, MCF-7, and MDA-MB-468 cells

| | | ADC- | ADC- | ADC- | ADC- | ADC- | ADC- | Cpd | Cpd | Cpd | | | Trop2- | Trop2- |
|---|---|---|---|---|---|---|---|---|---|---|---|---|---|---|
| | | 4 | 5 | 6 | 7 | 8 | 9 | 2 | 3 | 4 | Sacituzumab | Trodelvy | 1 | 2 |
| SKBR- | ReIC₅₀ | 2.883 | 0.327 | 0.053 | 2.402 | 1.572 | 0.173 | 5.811 | 22.18 | 8.581 | NA | 0.846 | NA | NA |
| 3 | (nM) | | | | | | | | | | | | | |
| | Bottom | 0 | 0 | 9.556 | 3.115 | 0 | 1.855 | 3.489 | 2.951 | 0 | 3.225 | 0 | 1.44 | 2.676 |
| | (%) | | | | | | | | | | | | | |
| | Top | 81.31 | 77.2 | 79.35 | 73.63 | 79.59 | 78.99 | 82.2 | 83.07 | 81.34 | 9.741 | 78.16 | 19.64 | 8.888 |
| | (%) | | | | | | | | | | | | | |
| NCI- | ReIC₅₀ | 17.24 | 0.811 | 0.186 | 19.15 | 1.61 | 0.405 | 32.41 | 61.71 | 22.17 | NA | 5.389 | 90.78 | NA |
| N87 | (nM) | | | | | | | | | | | | | |
| | Bottom | 0 | 0 | 3.489 | 0 | 0.958 | 1.245 | 0 | 0 | 0 | NA | 0 | 0 | NA |
| | (%) | | | | | | | | | | | | | |
| | Top | 71.41 | 49.97 | 55.05 | 69.5 | 55.42 | 51.07 | 60.82 | 69.68 | 60.27 | NA | 76.68 | 8.204 | NA |
| | (%) | | | | | | | | | | | | | |
| MCF- | ReIC₅₀ | 192 | 0.665 | 0.11 | 2.57 | 1.238 | 0.401 | 63.97 | 11.09 | 12.72 | NA | 40.49 | NA | NA |
| 7 | (nM) | | | | | | | | | | | | | |
| | Bottom | 0.026 | 0 | 0 | 0 | 0 | 0 | 0 | 0 | 0 | 0 | 0 | NA | NA |
| | (%) | | | | | | | | | | | | | |
| | Top | 100 | 40.37 | 50.87 | 43.21 | 34.81 | 50.55 | 38.44 | 28.93 | 37.63 | NA | 35.94 | NA | NA |
| | (%) | | | | | | | | | | | | | |
| MDA- | ReIC₅₀ | 7.426 | 1.228 | 0.286 | 9.485 | 2.98 | 0.655 | 14.82 | NA | 24.79 | NA | 2.333 | NA | 1.168 |
| MB- | (nM) | | | | | | | | | | | | | |
| 468 | Bottom | 0 | 0 | 0 | 0 | 0 | 0 | 0 | 0 | 0 | NA | 0 | NA | 0 |
| | (%) | | | | | | | | | | | | | |
| | Top | 88.37 | 73.96 | 76.2 | 85.98 | 74.38 | 74.89 | 83.21 | 54.17 | 78.73 | NA | 79.13 | NA | −4.402 |
| | (%) | | | | | | | | | | | | | |

### Example 24: In Vivo Therapeutic Efficacy of Anti-Trop2 ADCs in NCI-N87 Cell Xenograft Mouse Models

On day 0, NCI-N87 cells (1×10⁷ with 1:1 Matrigel) suspended in 0.2 mL PBS supplement with 50% (v/v) matrigel were injected subcutaneously in the right upper flank of 6- to 8-wk-old BALB/c female nude mice (purchased from Beijing Vital River Laboratory Animal Technology Co., Ltd). On day 6, mice were randomized to 8 groups (n=6) when the average tumor volume was 178 mm³. Treatments were initiated on the very same day (defined as PG-D0). Mice at Trodelvy group received 5.0 mg/kg (i.v., BIW) for 3 wk; mice at the ADC-5 treatment groups were injected with 1.0, 2.5, and 5.0 mg/kg (i.v., BIW) ADC-5 for 3 wk; mice at ADC-6 treatment groups were injected (i.v.) with 1.0 and 2.5 mg/kg ADC-6 weekly (QW) for three doses, or 5.0 mg/kg (single dose at day PG-D0). The tumor volumes and body weights were measured and recorded twice a week. On the termination day 28 (defined as PG-D28), tumor weight were dissected and measured. Tumor growth inhibition rate (%) was calculated as (TV_{CR}−TV_{TR})/TV_{CR} (%), where TV_{CR} and TV_{TR} are the relative tumor volumes of the vehicle control group and the experimental groups, respectively. Mice were euthanized and deemed to have succumbed to disease once tumors grew greater than 2,500 mm³. GraphPad Prism 6.0 Software was used for statistical analysis with a one-way ANOVA.

The results are shown in FIG. 7 and Table 5. The mice in all three ADC-5 treatment groups (1.0, 2.5, and 5.0 mg/kg, BIW*3) demonstrated a significantly better antitumor response than the mice in Trodelvy (5.0 mg/kg, BIW*3) treatment group. In addition, the mice in ADC-5 treatment groups at middle (2.5 mg/kg, BIW) and high (5.0 mg/kg, BIW) doses showed complete tumor response (CR) with the tumor growth inhibition (TGI) on day 28 of 100.37% and 106.43%, respectively. For ADC-6 treatment group, the results of middle dose (2.5 mg/kg, QW*3) and high dose (5.0 mg/kg, single dose) with lower frequency and/or dosage amount indicated that ADC-6 also exhibited a great tumor inhibitory efficacy, though ADC-6 at low dose (1.0 mg/kg, QW*3) presented a slightly weaker anti-tumor efficacy due to the less frequency and amount of the dosage than Trodelvy. Meanwhile, the results of body weight change indicated no obvious potential toxic risk of ADC-5 and ADC-6 in this in vivo efficacy study, which further provided solid evidence for an expanded therapeutic window of ADC-5 and ADC-6 than Trodelvy (FIG. 7B).

TABLE 5
Tumor growth inhibition rate (TGI, %) of exemplary anti-Trop2
ADCs in gastric cancer NCI-N87 cell xenograft mouse model

| Treatment | TGI (%) in days after administration | | | | | | | |
|---|---|---|---|---|---|---|---|---|
| groups | Day 3 | Day 7 | Day 10 | Day 14 | Day 17 | Day 21 | Day 24 | Day 28 |
| Vehicle, IV, | NA | NA | NA | NA | NA | NA | NA | NA |
| BIW*3 | | | | | | | | |
| Trodelvy, 5 mg/kg, | 44.83 | 44.43 | 39.67 | 42.59 | 49.05 | 55.07 | 55.32 | 54.53 |
| IV, BIW*3 | | | | | | | | |
| ADC-5, 1 mg/kg, | 16.91 | 27.26 | 30.28 | 35.03 | 49.34 | 56.15 | 61.09 | 65.29 |
| IV, BIW*3 | | | | | | | | |
| ADC-5, 2.5 | 19.92 | 56.81 | 86.82 | 90.48 | 94.44 | 98.25 | 100.24 | 100.37 |
| mg/kg, IV, BIW*3 | | | | | | | | |
| ADC-5, 5 mg/kg, | 100.35 | 92.02 | 111.91 | 113.31 | 110.87 | 109.52 | 108.01 | 106.43 |
| IV, BIW*3 | | | | | | | | |
| ADC-6, 1 mg/kg, | 26.92 | 5.17 | 15.35 | 19.94 | 28.53 | 36.40 | 42.09 | 45.37 |
| IV, QW*3 | | | | | | | | |
| ADC-6, 2.5 | 32.63 | 38.29 | 49.49 | 56.36 | 63.88 | 68.58 | 72.58 | 74.31 |
| mg/kg, IV, QW*3 | | | | | | | | |
| ADC-6, 5 mg/kg, | 71.22 | 59.23 | 83.25 | 89.27 | 92.14 | 91.48 | 90.88 | 88.49 |
| IV, single dose | | | | | | | | |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

The words “comprise”, “comprising”, “include”, “including”, and “includes” when used in this specification and in the following claims are intended to specify the presence of stated features, integers, components, or steps, but they do not preclude the presence or addition of one or more other features, integers, components, steps, or groups thereof.

What claimed is:

1. A polymeric scaffold of Formula (I) useful to conjugate with a targeting moiety: 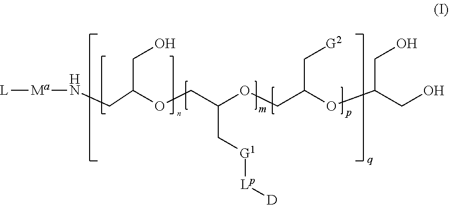
wherein:
the polymeric scaffold comprises linear polyglycerol;
L is a linking moiety comprising a functional group Wₚ that is capable of forming a covalent bond with the targeting moiety;
M^{a} is a stretcher connecting L to —NH— moiety;
each G¹ is independently a functional group connecting L^{p} to the linear polyglycerol;
each L^{p} is independently a drug release mechanism between a therapeutic agent moiety D and G¹;
each D is independently a therapeutic agent moiety;
each G2 is independently a functional group capable of converting into a charged state;
n is an integer from 0 to 1000;
m is an integer from 0 to 1000;
p is an integer from 0 to 1000; and
q is an integer from 0 to 1000.

2. The polymeric scaffold of claim 1, wherein Wₚ is capable of reacting with a functional group on the targeting moiety with a click reaction.

3. The polymeric scaffold of claim 2, wherein Wₚ is selected from the group consisting of:

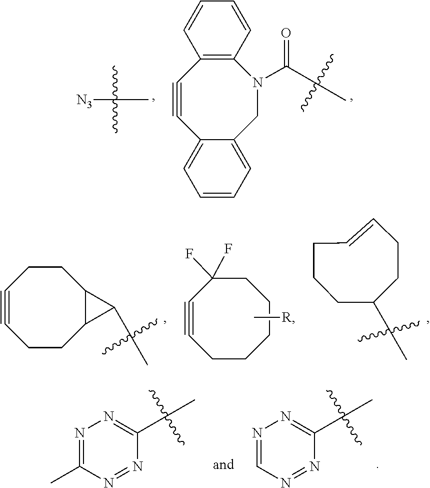

4. The polymeric scaffold of claim 1, wherein Wₚ is capable of reacting with amino acids on the targeting moiety.

5. The polymeric scaffold of claim 4, wherein the amino acids are natural amino acids, non-natural amino acids or combination thereof.

6. The polymeric scaffold of claim 5, wherein the natural amino acid comprises cysteine, lysine, tyrosine, aspartic acid and glutamic acid.

7. The polymeric scaffold of claim 4, wherein Wₚ is capable of reacting with one or more cysteines on the targeting moiety.

8. The polymeric scaffold of claim 7, wherein Wₚ is selected from the group 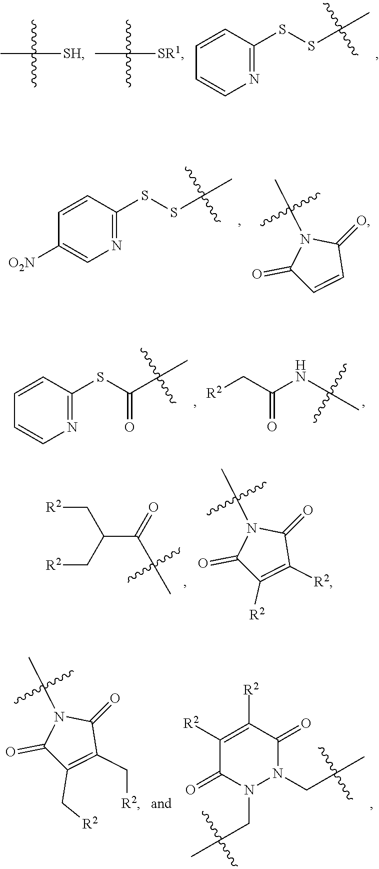
wherein R¹ is a sulfur protecting group, each R² is independently a leaving group.

9. The polymeric scaffold of claim 8, wherein each R² is independently selected from halo or R^{2a}C(O)O—, in which R^{2a} is hydrogen, an aliphatic, heteroaliphatic, cycloalkyl, heterocycloalkyl, aryl or heteroaryl.

10. The polymeric scaffold of claim 4, wherein Wₚ is capable of reacting with one or more lysines on the targeting moiety, in which each Wₚ is independently selected from the group consisting of:

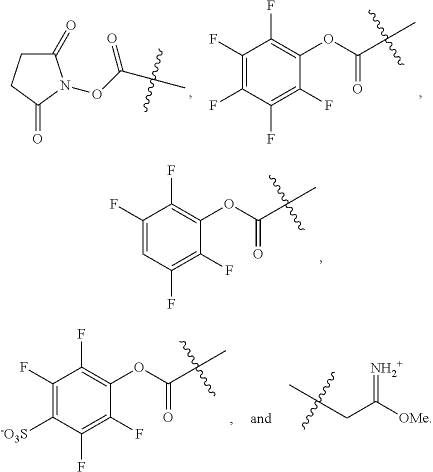

11. The polymeric scaffold of claim 4, wherein Wₚ is capable of reacting with one or more non-natural amino acids on the targeting moiety.

12. The polymeric scaffold of claim 11, wherein Wₚ is selected from:

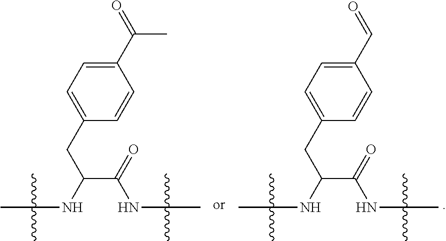 [SMILES: *NC(=O)C(Cc1ccc(C(C)=O)cc1)N* ; *NC(=O)C(Cc1ccc(C=O)cc1)N*]

13. The polymeric scaffold of claim 1, wherein M^{a} is selected from the group 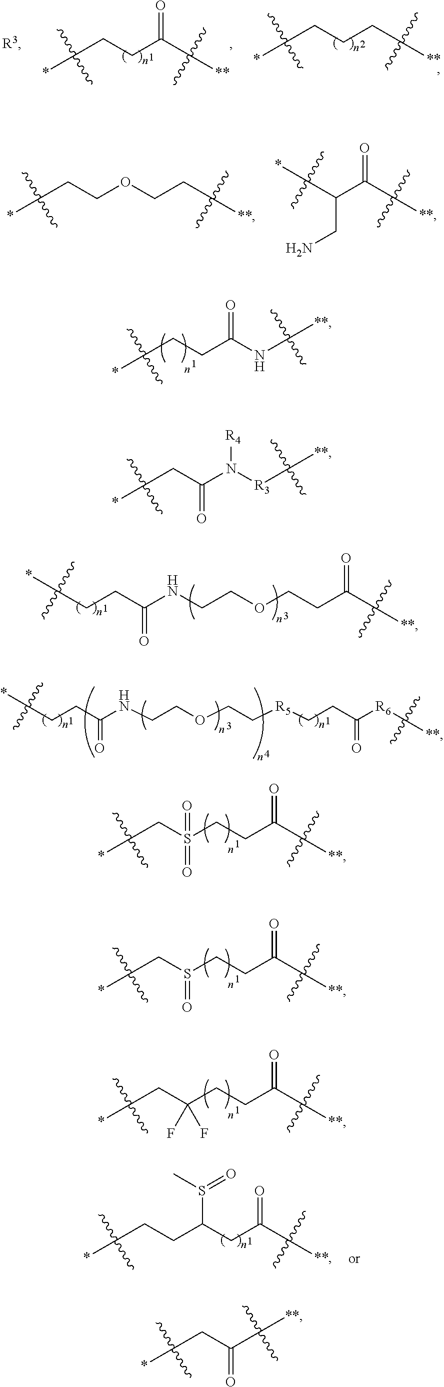
wherein * is the site covalently attached to L, ** is the site covalently attached to —NH— moiety,
R³ is C₁₋₁₀ alkyl, C₁₋₁₀ heteroalkyl, C₃₋₈ cycloalkyl, —O—(C₁₋₈ alkyl)-, aryl, —C₁₋₁₀ alkyl-aryl-, -aryl-C₁₋₁₀ alkyl-, —C₁₋₁₀ alkyl-(C₃₋₈ cycloalkyl)-, —(C₃₋₈ cycloalkyl-C₁₋₁₀ alkyl)-, 4- to 14-membered heterocycloalkyl, —C₁₋₁₀ alkyl-(4- to 14-membered heterocycloalkyl)-, -(4- to 14-membered heterocycloalkyl)-C₁₋₁₀ alkyl-, —C₁₋₁₀ alkyl-C(═O)—, —C₁₋₁₀ heteroalkyl-C(═O)—, —C₃₋₈ cycloalkyl-C(═O)—, —O—(C₁₋₈ alkyl)-C(═O)—, -aryl-C(═O)—, —C₁₋₁₀ alkyl-aryl-C(═O)—, -aryl-C₁₋₁₀ alkyl-C(═O)—, —C₁₋₁₀ alkyl-(C₃₋₈ cycloalkyl)-C(═O)—, —(C₃₋₈ cycloalkyl)-C₁₋₁₀ alkyl-C(═O)—, -4- to 14-membered heterocycloalkyl-C═(O)—, —C₁₋₁₀ alkyl-(4- to 14-membered heterocycloalkyl)-C(═O)—, -(4- to 14-membered heterocycloalkyl)-C₁₋₁₀ alkyl-C(═O)—, —C₁₋₁₀ alkyl-NH—, —C₁₋₁₀ heteroalkyl-NH—, —C₃₋₈ cycloalkyl-NH—, —O—(C₁₋₈ alkyl)-NH—, -aryl-NH—, —C₁₋₁₀ alkyl-aryl-NH—, -aryl-C₁₋₁₀ alkyl-NH—, —C₁₋₁₀ alkyl-(C₃₋₈ cycloalkyl)-NH—, —(C₃₋₈ cycloalkyl)-C₁₋₁₀ alkyl-NH—, -4- to 14-membered heterocycloalkyl-NH—, —C₁₋₁₀ alkyl-(4- to 14-membered heterocycloalkyl)-NH—, -(4- to 14-membered heterocycloalkyl)-C₁₋₁₀ alkyl-NH—, —C₁₋₁₀ alkyl-S—, —C₁₋₁₀ heteroalkyl-S—, —C₃₋₈ cycloalkyl-S—, —O—C₁₋₈ alkyl-S—, -aryl-S—, —C₁₋₁₀ alkyl-aryl-S—, -aryl-C₁₋₁₀ alkyl-S—, —C₁₋₁₀ alkyl-(C₃₋₈ cycloalkyl)-S—, —(C₃₋₈ cycloalkyl)-C₁₋₁₀ alkyl-S—, -4- to 14-membered heterocycloalkyl-S—, —C₁₋₁₀ alkyl-(4- to 14-membered heterocycloalkyl)-S—, or -(4- to 14-membered heterocycloalkyl)-C₁₋₁₀ alkyl-S—;
each R⁴ independently is hydrogen, C₁₋₆ alkyl, C₆₋₁₀ aryl, C₃₋₈ cycloalkyl, —COOH or —COO—C₁₋₆ alkyl;
R⁵ is —C(O)—NR^{5a} or —NR^{5a}—C(O)—;
R^{5a} is hydrogen, C₁₋₆ alkyl, C₆₋₁₀ aryl, C₃₋₈ cycloalkyl, —COOH or —COO—C₁₋₆ alkyl;
R⁶ is a bond or —NR^{6a}—(CR^{6b}R^{6c})—C(O)—;
R^{6a} is hydrogen, C₁₋₆ alkyl, C₆₋₁₀ aryl, C₃₋₈ cycloalkyl, —COOH or —COO—C₁₋₆ alkyl;
each R^{6b} and R^{6c} independently is hydrogen, C₁₋₆ alkyl, C₆₋₁₀ aryl, hydroxylated C₆₋₁₀ aryl, polyhydroxylated C₆₋₁₀ aryl, 5- to 12-membered heterocycloalkyl, C₃₋₈ cycloalkyl, hydroxylated C₃₋₈ cycloalkyl, polyhydroxylated C₃₋₈ cycloalkyl or a side chain of a natural or unnatural amino acid;
each n¹ independently is an integer from 0 to 6;
n² is an integer from 0 to 8;
each n³ independently is an integer from 1 to 6; and
n⁴ is an integer from 1 to 4.

14. The polymeric scaffold of claim 13, wherein M^{a} is selected from the group consisting of:

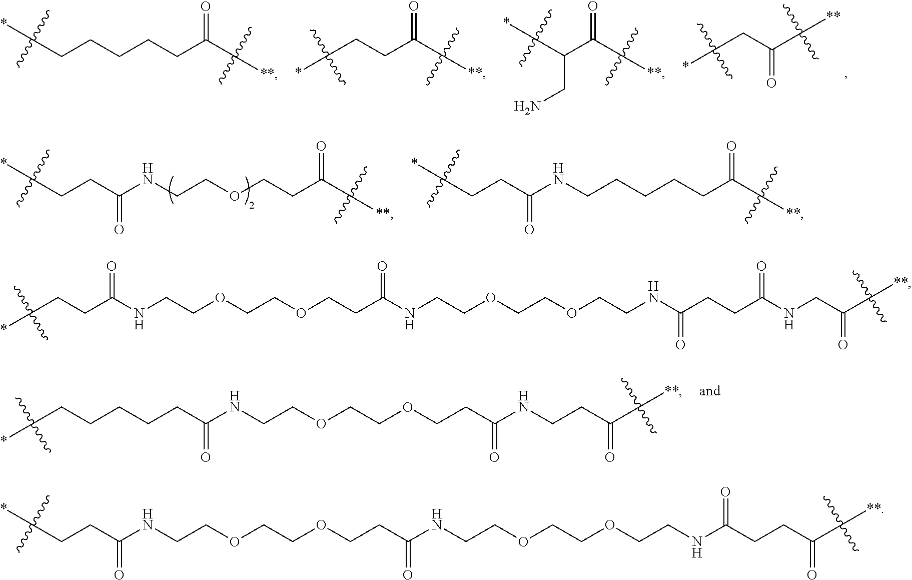

15. The polymeric scaffold of claim 1, wherein G¹ is selected from the group consisting of: 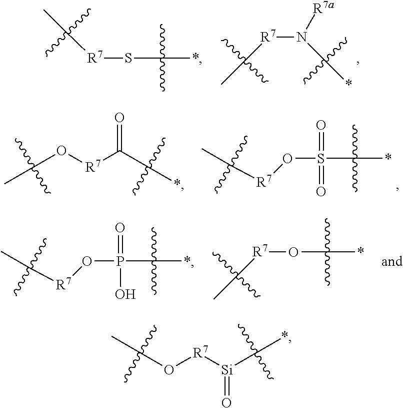
wherein * is the site covalently attached to L^{p}, each R⁷ is independently selected from a direct bond, alkyl, cycloalkyl, heterocycloalkyl, aryl, or heteroaryl, and R^{7a} is selected from hydrogen, an aliphatic, heteroaliphatic, cycloalkyl, or heterocycloalkyl moiety.

16. The polymeric scaffold of claim 1, wherein G¹ is selected from the group consisting of: 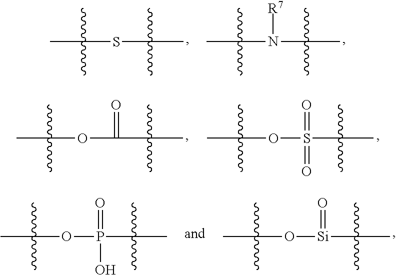
wherein R⁷ is selected from hydrogen, an aliphatic, heteroaliphatic, cycloalkyl, or heterocycloalkyl moiety.

17. The polymeric scaffold of claim 1, wherein each Lₚ independently comprises a labile structure.

18. The polymeric scaffold of claim 17, wherein the labile structure is selected from hydrolytically labile structures or enzymatic labile structures.

19. The polymeric scaffold of claim 18, wherein the hydrolytically labile structure is selected from the group consisting of: 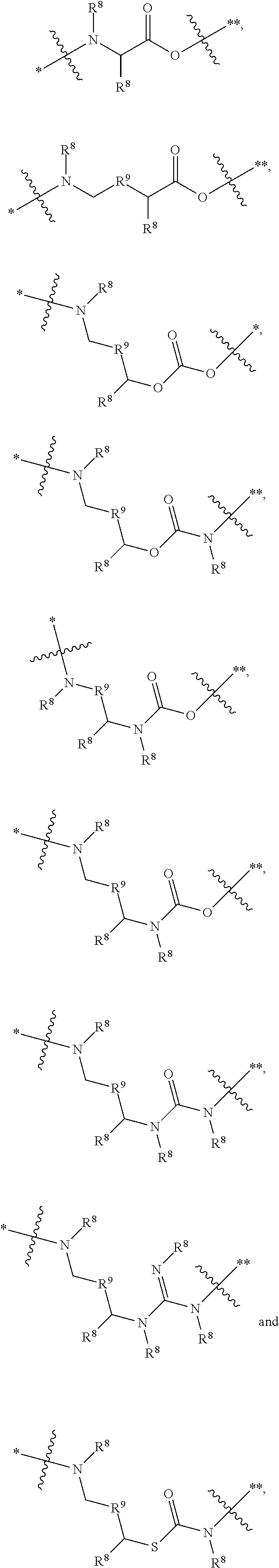
wherein * is the site covalently attached to G¹, ** is the site covalently attached to D, R⁸ is selected from hydrogen, alky or aryl, R⁹ is selected from aliphatic, heteroaliphatic, cycloalkyl, heterocycloalkyl, aryl, or heteroaryl.

20. The polymeric scaffold of claim 18, wherein the hydrolytically labile structure is selected from the group consisting of: 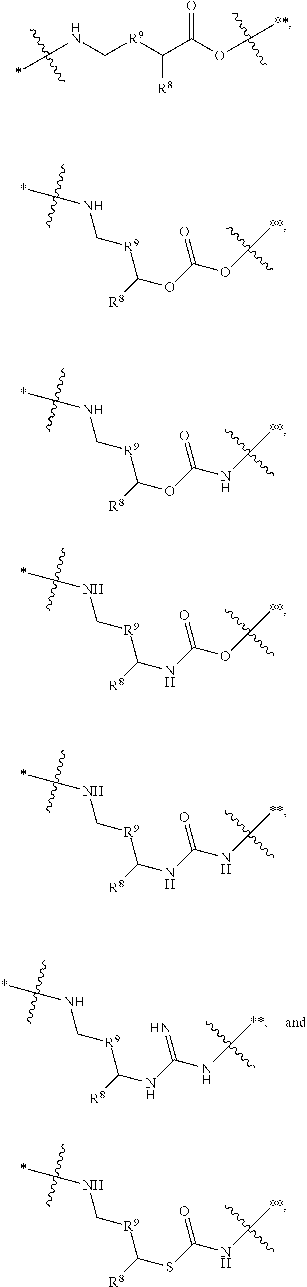
wherein * is the site covalently attached to G¹, ** is the site covalently attached to D, R⁸ is selected from hydrogen, alky or aryl, R⁹ is selected from aliphatic, heteroaliphatic, cycloalkyl, heterocycloalkyl, aryl, or heteroaryl.

21. The polymeric scaffold of claim 20, wherein G¹ is 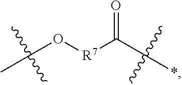
wherein * is the site covalently attached to L^{p}, R⁷ is alkyl.

22. The polymeric scaffold of claim 21, wherein -G¹-L^{p}-D is

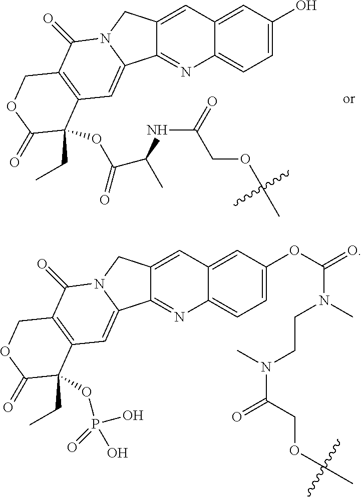 [SMILES: *OCC(=O)N[C@@H](C)C(=O)O[C@]1(CC)C(=O)OCc2c1cc1n(c2=O)Cc2cc3cc(O)ccc3nc2-1 ; *OCC(=O)N(C)CCN(C)C(=O)Oc1ccc2nc3c(cc2c1)Cn1c-3cc2c(c1=O)COC(=O)[C@@]2(CC)OP(=O)(O)O]

23. The polymeric scaffold of claim 17, wherein the enzymatic labile structure is liable to enzymes selected from Cathepsin B, phosphatase, sulfatase, or glucuronidase.

24. The polymeric scaffold of claim 19, wherein the enzymatic labile structure is liable to cathepsin B and is selected from —Z— or 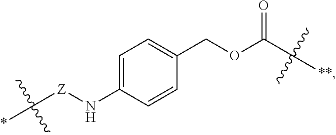
wherein Z is a substrate for cathepsin B comprising 2 to 4 amino acids.

25. The polymeric scaffold of claim 24, wherein G¹ is 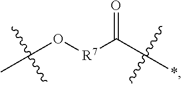
wherein * is the site covalently attached to L^{p}, R⁷ is alkyl.

26. The polymeric scaffold of claim 24, wherein -G¹-L^{p}-D is

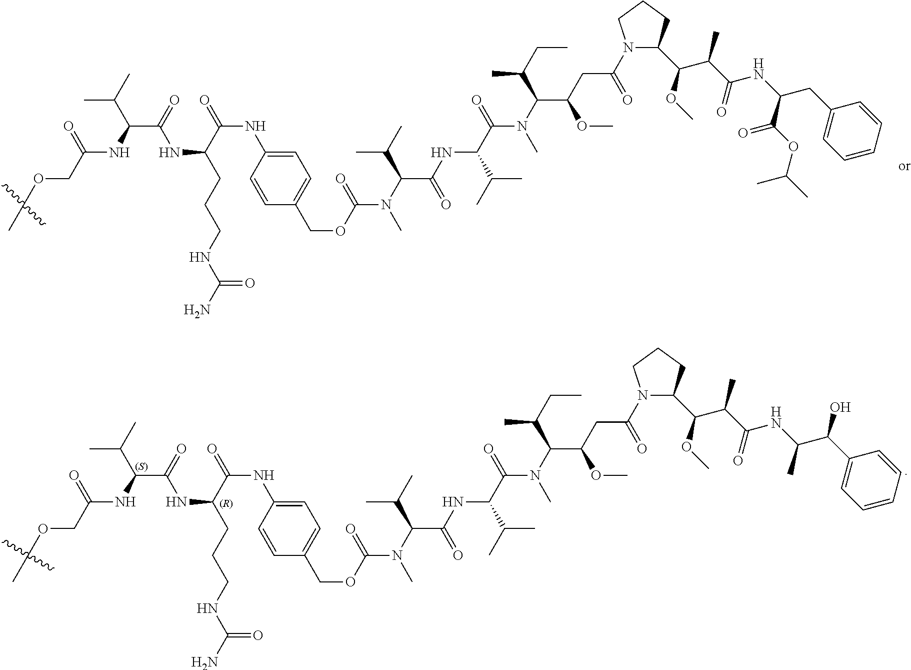

27. The polymeric scaffold of claim 23, wherein the enzymatic labile structure is liable to glucuronidase and is 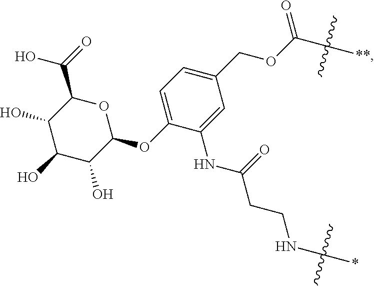
wherein * is the site covalently attached to G¹, ** is the site covalently attached to D.

28. The polymeric scaffold of claim 27, wherein G¹ is 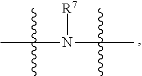
wherein R⁷ is selected from hydrogen, aliphatic, heteroaliphatic, cycloalkyl, heterocycloalkyl, aryl or heteroaryl.

29. The polymeric scaffold of claim 27, wherein G¹ is 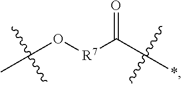
wherein * is the site covalently attached to L^{p}, R⁷ is alkyl.

30. The polymeric scaffold of claim 27, wherein -L^{p}-D is selected from:

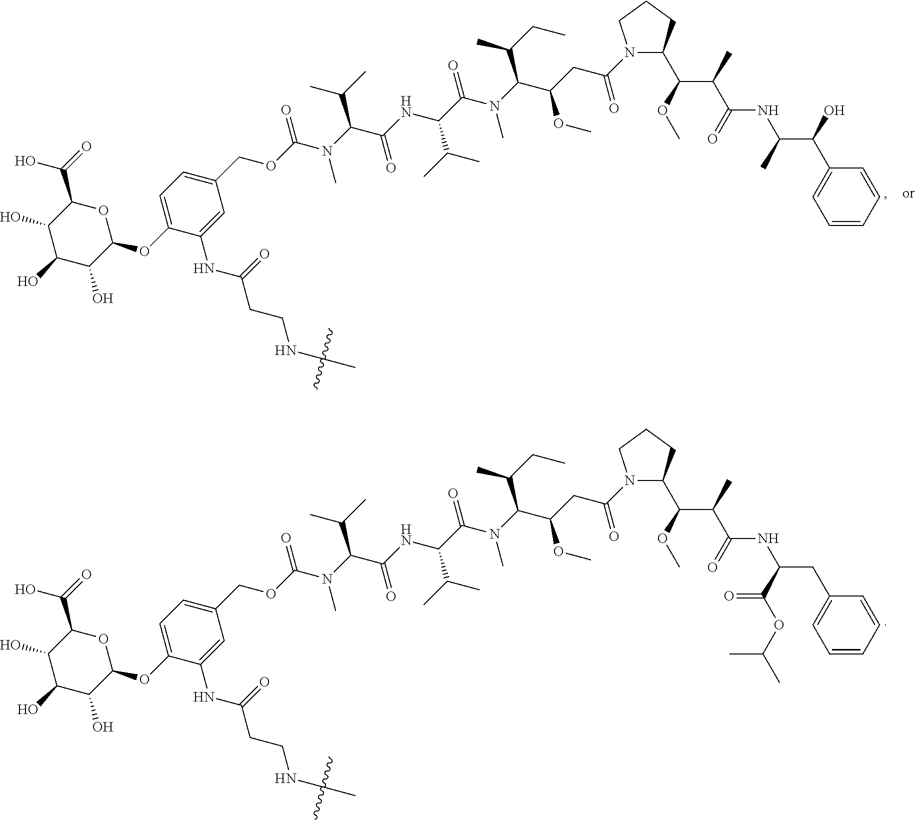 [SMILES: *NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)ccc1O[C@@H]1O[C@H](C(=O)O)[C@@H](O)[C@H](O)[C@H]1O ; *NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)C(=O)OC(C)C)OC)C(C)C)C(C)C)ccc1O[C@@H]1O[C@H](C(=O)O)[C@@H](O)[C@H](O)[C@H]1O]

31. The polymeric scaffold of claim 27, wherein -G¹-L^{p}-D is selected from:

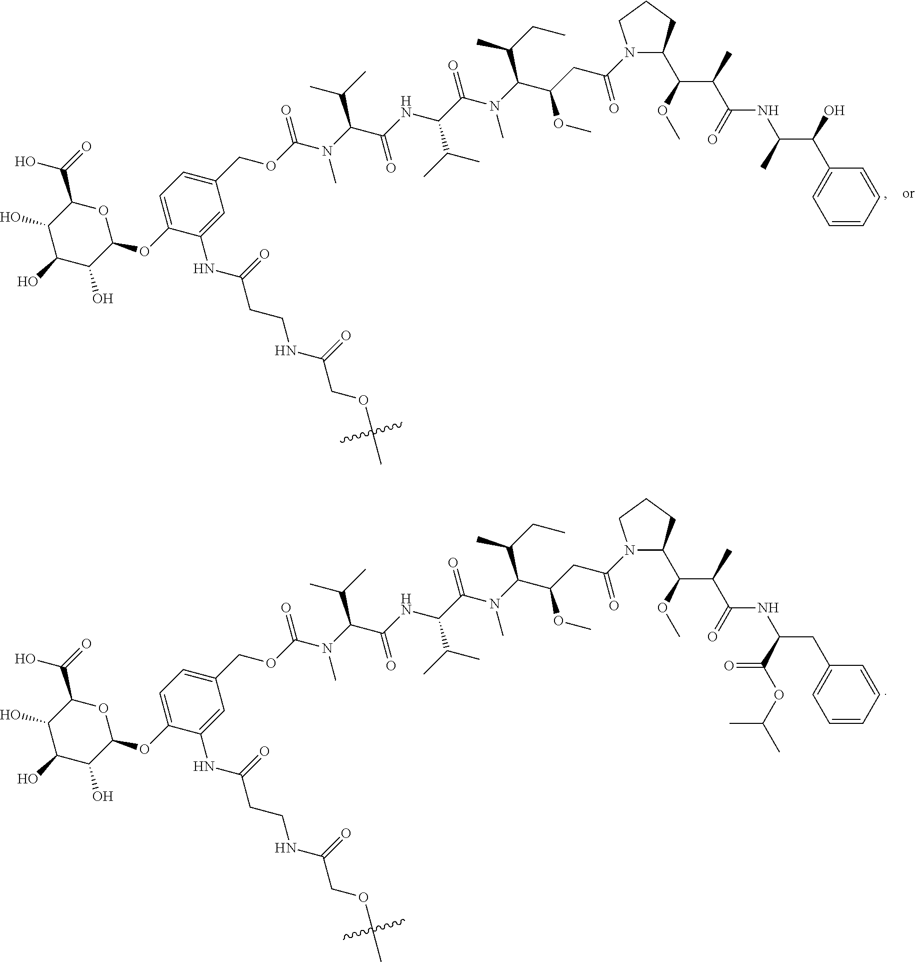 [SMILES: *OCC(=O)NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)ccc1O[C@@H]1O[C@H](C(=O)O)[C@@H](O)[C@H](O)[C@H]1O ; *OCC(=O)NCCC(=O)Nc1cc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)C(=O)OC(C)C)OC)C(C)C)C(C)C)ccc1O[C@@H]1O[C@H](C(=O)O)[C@@H](O)[C@H](O)[C@H]1O]

32. The polymeric scaffold of claim 23, wherein the enzymatic labile structure is liable to phosphatase and is selected from 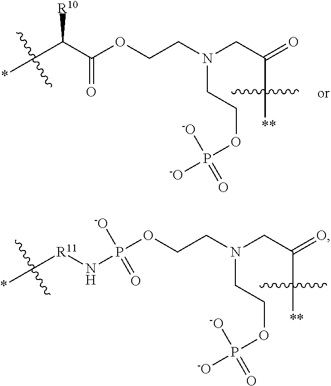
wherein * is the site covalently attached to G¹, ** is the site covalently attached to D, each of R¹⁰ and R¹¹ is independently hydrogen, an aliphatic, heteroaliphatic, cycloalkyl, heterocycloalkyl, aryl or heteroaryl.

33. The polymeric scaffold of claim 32, wherein G¹ is

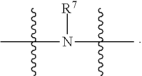

34. The polymeric scaffold of claim 33 wherein -G¹-L^{p}-D is selected from the group consisting of:

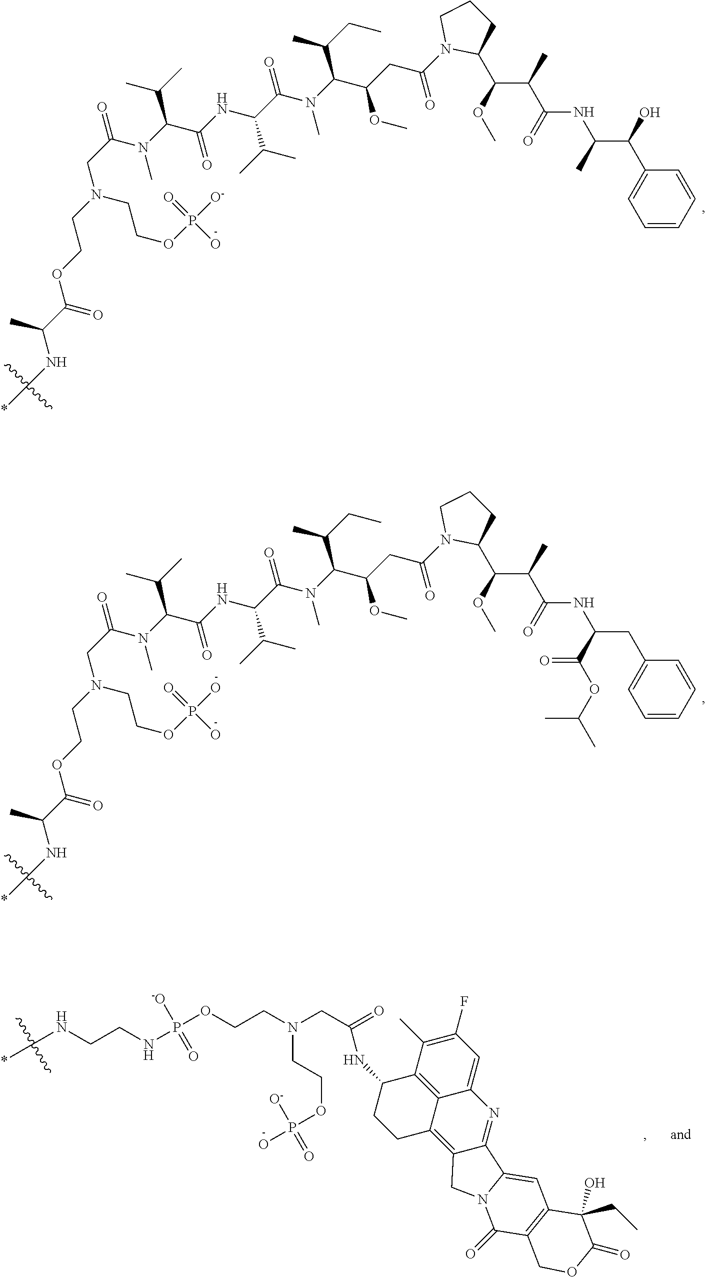

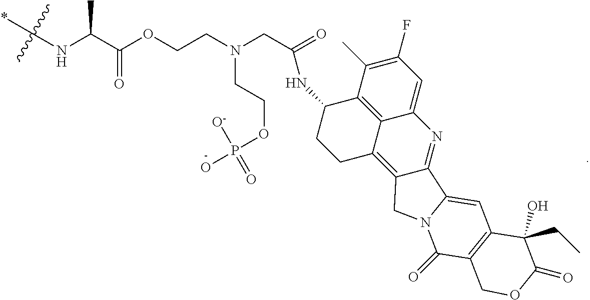

35. The polymeric scaffold of claim 23, wherein the enzymatic labile structures are liable to sulfatase and is 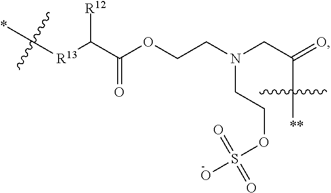
wherein * is the site covalently attached to G¹, ** is the site covalently attached to D, each of R¹² and R¹³ is independently hydrogen, an aliphatic, heteroaliphatic, cycloalkyl, heterocycloalkyl, aryl or heteroaryl.

36. The polymeric scaffold of claim 35, wherein G¹ is 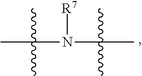
wherein R⁷ is selected from hydrogen, an aliphatic, heteroaliphatic, cycloalkyl, heterocycloalkyl, aryl or heteroaryl.

37. The polymeric scaffold of claim 35, wherein G¹ is 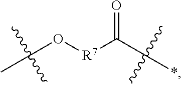
wherein * is the site covalently attached to L^{p}, R⁷ is alkyl.

38. The polymeric scaffold of claim 37, wherein -G¹-L^{p}-D is:

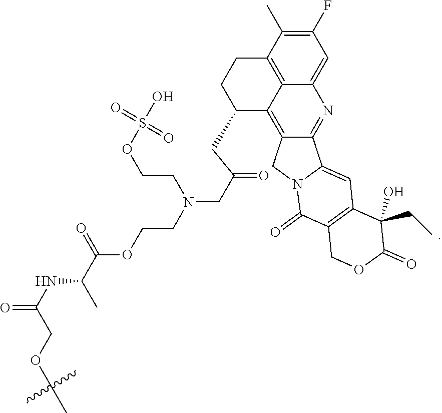 [SMILES: *OCC(=O)N[C@@H](C)C(=O)OCCN(CCOS(=O)(=O)O)CC(=O)C[C@@H]1CCc2c(C)c(F)cc3nc4c(c1c23)Cn1c-4cc2c(c1=O)COC(=O)[C@]2(O)CC]

39. The polymeric scaffold of claim 1, wherein
n is an integer from 1 to 100;
m is an integer from 1 to 100; and
p is an integer from 1 to 50.

40. The polymeric scaffold of claim 1, wherein the therapeutic agent has antiproliferative activity against a target cell or pathway.

41. The polymeric scaffold of claim 40, wherein the antiproliferative activity is selected from cytostatic and/or cytotoxic activity.

42. The polymeric scaffold of claim 1, wherein the therapeutic agent is selected from anti-cancer substances, cytotoxic drugs, radionuclides, vitamins, anti-AIDS substances, antibiotics, immunosuppressants, immunomodulatory compounds, therapeutic RNAs, anti-viral substances, enzyme inhibitors, neurotoxins, opioids, hypnotics, anti-histamines, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, antisecretory factors, anticoagulants and/or antithrombotic agents, local anesthetics, ophthalmics, prostaglandins, anti-depressants, anti-psychotic substances, anti-emetics, imaging agents.

43. The polymeric scaffold of claim 1, wherein the therapeutic agent comprises amino acid-based molecules.

44. The polymeric scaffold of claim 43, wherein the amino acid-based molecules comprise peptides, polypeptides, enzymes, antibodies, immunoglobulins, or functional fragments thereof.

45. The polymeric scaffold of claim 1, wherein the therapeutic agent has a chemically reactive group.

46. The polymeric scaffold of claim 45, wherein the chemically reactive group comprises —COOH, primary amine, secondary amine-NHR, —OH, —SH, —C(O)H, C(O)R¹⁴, —C(O)NHR¹⁵, C(S)OH, —S(O)₂OR¹⁵, —P(O)₂OR¹⁵, —CN, —NC or —ONO, in which R¹⁴ is selected from an aliphatic, heteroahphatic, cycloalkyl, heterocycloalkyl, aryl or heteroaryl, and R¹⁵ is selected from a hydrogen, aliphatic, heteroaliphatic, cycloalkyl, heterocycloalkyl, aryl or heteroaryl.

47. The polymeric scaffold of claim 1, wherein G² is selected from the group consisting of: 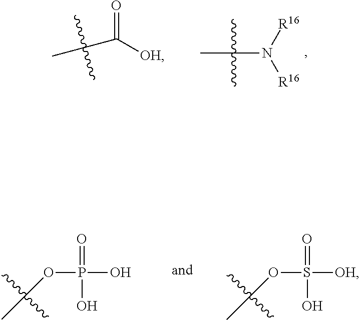
wherein each R¹⁶ is independently hydrogen, an aliphatic, heteroaliphatic, cycloalkyl, heterocycloalkyl, aryl or heteroaryl.

48. The polymeric scaffold of claim 1, wherein G² is

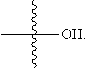 [SMILES: *O]

49. A polymeric scaffold of Formula (II): 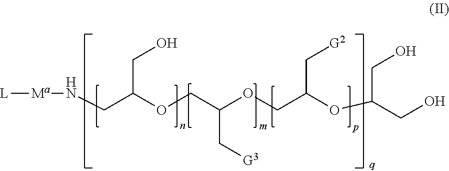
wherein,
the polymeric scaffold comprises linear polyglycerol;
L is a linking moiety comprising a functional group Wₚ that is capable of forming a covalent bond with the targeting moiety;
M^{a} is a stretcher connecting L to —NH— moiety;
each G² is independently a functional group capable of converting into a charged state;
each G³ independently comprises a functional group capable of reacting with a reactive group in a drug release mechanism to connect the drug release mechanism to the linear polyglycerol;
n is an integer from 0 to 1000;
m is an integer from 0 to 1000;
p is an integer from 0 to 1000; and
q is an integer from 0 to 1000.

50. The polymeric scaffold of claim 49, wherein
n is 2;
m is 2; and
p is 2.

51. The polymeric scaffold of claim 49, wherein q is an integer from 3 to 5.

52. The polymeric scaffold of claim 49, wherein G² and G³ are independently selected from the group insisting of: 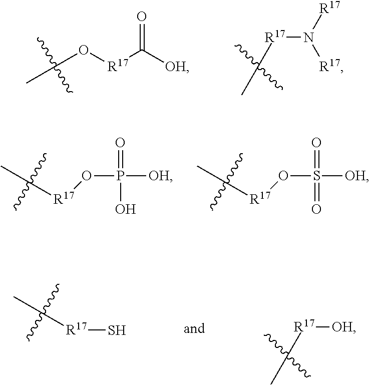
in which, each R¹⁷ is independently selected from a direct bond, alkyl, cycloalkyl, heterocycloalkyl, aryl, or heteroaryl.

53. The polymeric scaffold of claim 49, having a structure of Formula (IIa) or (IIb):

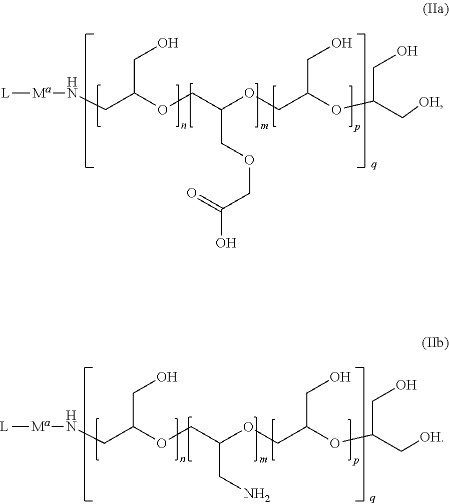

54. The polymeric scaffold of claim 52, having a structure of Formula (IIc) or (IId): 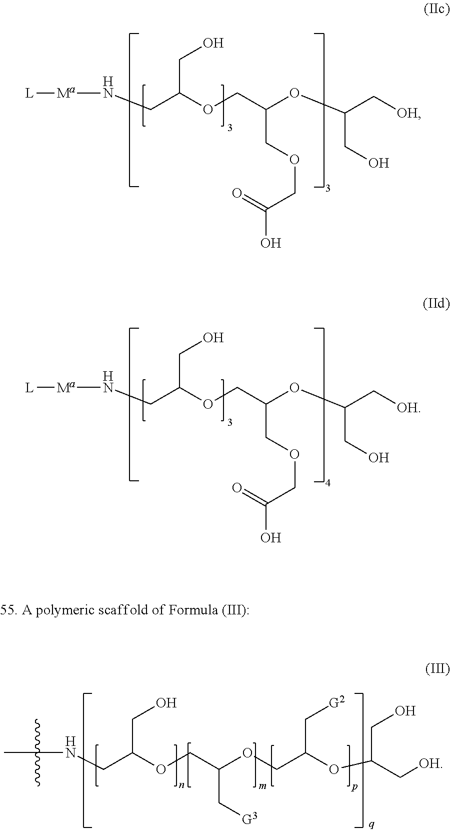
wherein,
the polymeric scaffold comprises linear polyglycerol;
each G² is independently a functional group capable of converting into a charged state;
each G³ independently comprises a functional group capable of reacting with a reactive group in a drug release mechanism to connect the drug release mechanism to the linear polyglycerol;
n is an integer from 0 to 1000;
m is an integer from 0 to 1000;
p is an integer from 0 to 1000; and
q is an integer from 0 to 1000.

56. The polymeric scaffold of claim 55, wherein
n is 2;
m is 2; and
p is 2.

57. The polymeric scaffold of claim 55, wherein q is an integer from 3 to 5.

58. The polymeric scaffold of claim 55, wherein G² and G³ are independently selected from the group insisting of: 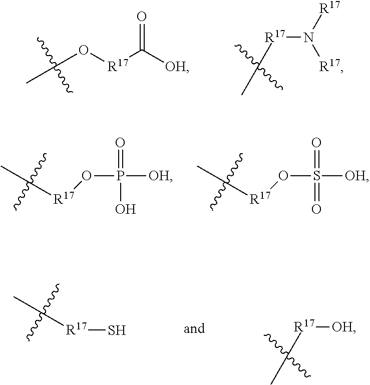
in which, each R¹⁷ is independently selected from a direct bond, alkyl, cycloalkyl, heterocycloalkyl, aryl or heteroaryl.

59. The polymeric scaffold of claim 55, having a structure of Formula (IIIa) or (IIIb):

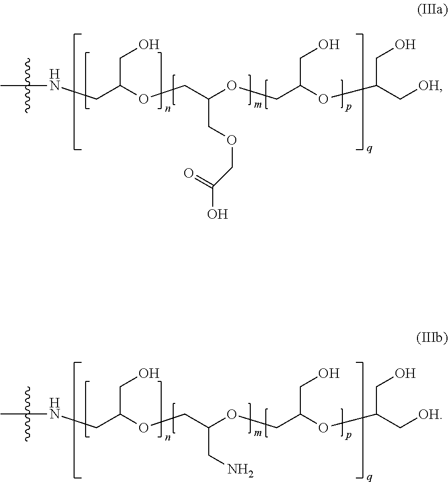

60. The polymeric scaffold of claim 58, having a structure of Formula (IIIc) or (IIId)

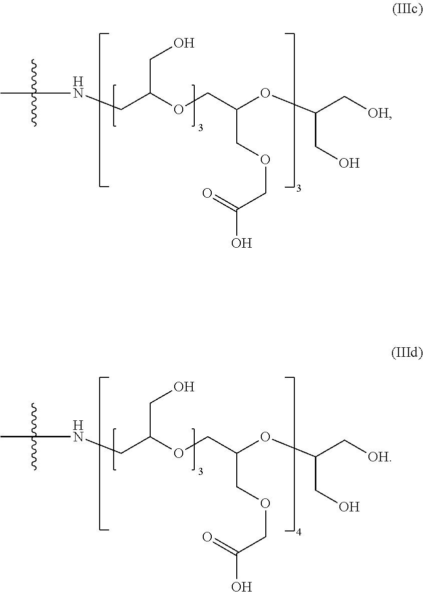

61. The polymeric scaffold of claim 55, having a weight average molecular weight Mw of 1-100000.

62. The polymeric scaffold of claim 55, having a weight average molecular weight Mw of 10000-15000.

63. The polymeric scaffold of claim 55, having a weight average molecular weight Mw of 5000-10000.

64. The polymeric scaffold of claim 55, having a PDI of less than 1.5.

65. A polymeric scaffold of Formula (IV): 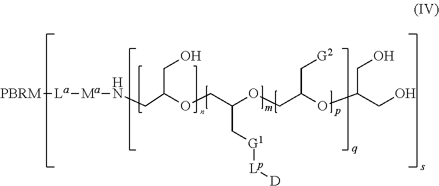
wherein,
the polymeric scaffold comprises linear polyglycerol;
each L^{a} is independently a divalent moiety connecting the targeting moiety to M^{a};
each M^{a} is independently a stretcher connecting L^{a} to —NH— moiety;
each G¹ is independently a functional group connecting L^{p} to the linear polyglycerol;
each L^{p} is independently a drug release mechanism between a fragment of a therapeutic agent D and G¹;
each D is independently a fragment of a therapeutic agent;
each G² is independently a functional group capable of converting into a charged state;
n is an integer from 0 to 1000;
m is an integer from 0 to 1000;
p is an integer from 0 to 1000;
q is an integer from 0 to 1000; and
s is an integer from 1 to 8.

66. The polymeric scaffold of claim 65, wherein the targeting moiety is an antibody and/or fragment thereof.

67. The polymeric scaffold of claim 65, wherein the targeting moiety is an antibody IgG1, IgG2, IgG3, and IgG4.

68. The polymeric scaffold of claim 65, wherein the targeting moiety is selected from the group consisting of a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer, a multispecific antibody, a camelized single domain antibody, a nanobody, a domain antibody, or a bivalent domain antibody.

69. A pharmaceutical composition comprising one or more of the polymeric scaffold according to any one of claims 1-48 and 65-68 and an acceptable carrier.

70. A method of treating a disorder in a subject in need thereof, the method comprising administering to the subject a therapeutic effective amount of the polymeric scaffold according to any one of claims 1-48 and 65-68 or the pharmaceutical composition according to claim 69.
